(19) United States
(12) Patent Application Publication
(10) Pub. No.: US 2023/0070731 A1
(43) Pub. Date: Mar. 9, 2023

(54) COMPOSITIONS FOR SMALL MOLECULE CONTROL OF PRECISE BASE EDITING OF TARGET NUCLEIC ACIDS AND METHODS OF USE THEREOF

(71) Applicant: THE TRUSTEES OF THE UNIVERSITY OF PENNSYLVANIA, Philadelphia, PA (US)

(72) Inventors: Rahul Kohli, Penn Valley, PA (US); Junwei Shi, Philadelphia, PA (US); Kiara Berrios, Somerville, MA (US)

(73) Assignee: THE TRUSTEES OF THE UNIVERSITY OF PENNSYLVANIA, Philadelphia, PA (US)

(21) Appl. No.: 17/795,191

(22) PCT Filed: Jan. 20, 2021
(86) PCT No.: PCT/US21/14252

§ 371 Date: Jul. 25, 2022

Related U.S. Application Data
(60) Provisional application No. 62/965,886, filed on Jan. 25, 2020.
(60) Provisional application No. 62/966,303, filed on Jan. 27, 2020.

(51) Int. Cl. C12N 9/78; C12N 15/11; C12N 9/22; C12N 15/62; C12N 15/90

(52) U.S. Cl. CPC C12N 9/78; C12N 15/11; C12N 9/22; C12Y 305/04004; C12N 15/62; C12N 15/907; C12N 2310/20; C12N 2800/80; C07K 2319/01

(57) ABSTRACT

Compositions and methods for small molecule control of precise base editing are disclosed.

### CROSS REFERENCE TO RELATED APPLICATIONS

This application claims priority of U.S. Provisional application Nos. 62/965,886 and 62/966,303 filed Jan. 25, 2020 and Jan. 27, 2020 respectively, the entire contents being incorporated herein by reference as though set forth in full.

### INCORPORATION-BY-REFERNCE OF MATERIAL SUBMITTED IN ELECTRONIC FORM

Incorporated herein by reference in its entirety is the Sequence Listing submitted via EFS-Web as a text file named SEQLIST_UPNK102.txt., created Jan. 20, 2021 and having a size of 235,835 bytes.

### FIELD OF THE INVENTION

This invention relates to the fields of gene therapy and base editing. More specifically, the invention provides split DNA deaminase encoding constructs which exhibit controllable and efficient base editing while reducing undesirable off target effects. Methods employing such constructs and kits comprising the same, are also disclosed.

### BACKGROUND OF THE INVENTION

Several publications and patent documents are cited throughout the specification in order to describe the state of the art to which this invention pertains. Each of these citations is incorporated herein by reference as though set forth in full.

Base editing of the immunoglobulin locus by AID, the ancestral member of the AID/APOBEC family of cytosine deaminase enzymes, normally initiates maturation of antibody responses in B-cells, while APOBEC3 enzymes provide protection against retroviruses. Out of their physiological context, when DNA deaminases are directed towards a specific genomic locus by catalytically-impaired Cas9, their base editing activity can be used to introduce targeted mutations at a desired locus. While this system offers a potentially powerful means to edit the genome for biological or therapeutic purposes, base editors have at least two natural constraints that could limit their broader application. First, the enzymes have naturally evolved to be constrained deaminases with low overall catalytic activity, as hyperactivation is associated with increased oncogenic mutations. Second, AID/APOBECs are known to act outside of their targets, promoting cancer mutagenesis, chromosomal translocations, and resistance to chemotherapy. When the natural regulatory constraints are lost, overexpression of a functionally intact deaminase in a gene editing complex poses similar risks to the genome. In existing base editors, the DNA deaminases are targeted, but they are not regulated which increases undesirable off-target activity which is not mitigated by linking it to a targeting module like dCas9. As the deaminase is active, overexpressed and present in the nucleus, the active enzyme will be able to access ssDNA intermediates normally exposed in the process of DNA replication, transcription, and repair, much as it does in cancers. Indeed, an increase in genome-wide mutation at activation induced deaminase (AID) preferred hotspots has been shown with expression of AID-containing ZFN and TALE base editors, and recent work has shown widespread genome-wide action by the most commonly employed BE3 base editors.

Added concerns arise from evidence of off-target deaminase activity on RNA, highlighting the need to regulate where and when the deaminases are active. Although many biological goals can be achieved with current base editors, the therapeutic utility of base editing approaches in human patients will be limited if off target activity is not addressed.

It is clear that a need exists in the art for improved base editors whose activity can be regulated to permit action with greater precision at the targeted site with minimal off target effects.

### SUMMARY OF THE INVENTION

The present invention provides precise base editor complexes and methods of use thereof for efficient and controllable site-specific editing at sites of interest in targeted DNA and RNA sequences. The base editor complexes described herein comprise different protein modules which act in concert to effect inducible and specific gene editing. The modules are fused using appropriate linker sequences and comprise at least a targeting module (TM) which localizes the complex to a particular genomic site of interest. The tethered modifying module (MM) edits the local DNA. In certain aspects skewing downstream repair pathways via inclusion of accessory modules (MM_{X}) can improve efficiency. Via inclusion of a specific binding pair into the complex, the present invention provides for regulatory, small molecule control over based editors by exploiting knowledge of DNA deaminase structure and function to split DNA deaminases into inactive components that can only be reconstituted at the desired site of action. In other embodiments, both the targeting module and the modifying modules are split and reassembled upon dimerization of the specific binding pair. In yet another aspect, the complex comprises two distinct targeting molecules, e.g., two distinct dCas9/sgRNAs, for enhanced specificity, each of which is linked to one part of the split deaminase.

In one embodiment, a first fusion protein for precise small molecule control of targeted base editing comprising an optional accessory module, a targeting module, a first portion of a split deaminase operably linked to a first member of a specific binding pair, and a second fusion protein comprising a second portion of a split deaminase which is operably linked to a second member of a specific binding pair is provided, wherein said specific binding pair members dimerize upon contact with a dimerization agent causing two portions of the split deaminase enzyme to reform thereby resulting in formation of small molecule inducible base editor complex which edits a site of interest on a nucleic acid bound by the targeting module.

In another aspect, a first fusion protein comprising a first portion of a split deaminase, operably linked to a first portion of a split targeting module, said targeting module being operably linked to a first member of a specific binding pair, and a second fusion protein comprising a second portion of a split deaminase operably linked to a second portion of a split targeting module operably linked to a second specific binding pair member is provided, wherein said specific binding pair members dimerize upon contact with a dimerization agent, causing two portions of a split deaminase enzyme and the two portions of the targeting module to reform thereby resulting in formation of small molecule inducible base editor complex which edits a site of interest on a nucleic acid bound by the targeting module.

In another embodiment, a first fusion protein comprising a targeting module operably linked to a first member of a specific binding pair which is operably linked to a first portion of a split deaminase and second fusion protein comprising a second member of a specific binding pair, operably linked to a second portion of a split deaminase which is operably linked to a separate second targeting module. The two targeting modules are approximated close to one another at the nucleic acid target, with the specific binding pair members dimerizing upon contact with a dimerization agent, wherein dimerization causes two portions of a split deaminase enzyme to reform thereby resulting in formation of small molecule inducible base editor complex which edits a site of interest on a nucleic acid bound by the two co-localizing targeting modules with reduced off target effects.

In certain aspects, the targeting molecule is selected from nCas9, dCas9, dCas12, nCas12, xCas9, Cas13, transcription activator effector-like effectors (TALENs), and zinc finger nucleases (ZFNs), and comprises at least one sequence which directs said base editing complex to the site to be edited.

Deaminase proteins useful in the base editing complexes described herein can be selected from rat or human APOBEC1, human APOBEC3A, APOBEC3B, APOBEC3C, APOBEC3DE, APOBEC3F, APOBEC3G, Activation-induced cytidine deaminase (AID), CDA from lamprey, mutant version of Adenosine Deaminases (TadA) engineered to act on DNA, and Adenosine Deaminase acting on dsRNA (ADAR) or proteins having at least 90% identity with these proteins.

The fusion proteins may also comprise accessory molecules for reducing efficiency. Such molecules include, without limitation, UGI, 2x UGI, and μ-GAM.

In preferred embodiments the fusion proteins are present in a cell, and the cell is contacted with an effective amount of a dimerization agent, thereby causing the specific binding pair to dimerize. Specific binding pairs included in the base editing complex include, without limitation, FKBP and FRB wherein binding is induced by contact with dimerization agent rapamycin or a rapamycin analog, FKBP-F36V and FKBP-F36V wherein binding is induced by dimerization agent AP1903, BCLxl and scAZI, where binding is induced with dimerization agent ABT₇₃₇, and CRY2 and CIB1 where binding is induced by light. In other embodiments, the first and second binding pairs are GFP 1-10 and GFP11 wherein binding occurs spontaneously.

Another embodiment of the invention includes a method of deaminating one or more selected bases in a target nucleic acid comprising contacting the target nucleic acid with the fusion proteins and dimerization agent described above. Also provided are host cells comprising the fusion proteins encoding the base editing complexes of the invention.

In another aspect a composition comprising the fusion proteins described above in a suitable biological carrier.

The invention also provides one or more isolated nucleic acids encoding the fusion proteins described above. Exemplary nucleic acids encoding the base editing complexes of the invention are shown in FIGS. 13 and 14. In certain embodiments, the nucleic acids are present in an expression vector, such as a retroviral vector, an adenoviral vector, an adeno-associated viral vector, a lentiviral vector, and a plasmid vector. RNA transcripts encoding the fusion proteins described above are also provided.

The compositions of the invention can further comprise one or more of a liposome, a nanoparticle, a pharmaceutically acceptable carrier, and a buffer.

In yet another aspect, a method of deaminating one or more selected bases in a target nucleic acid is disclosed. An exemplary method comprises contacting a cell harboring the target nucleic acid with the base editing complex encoding nucleic acids described above under conditions where said complex is expressed, and a dimerization agent, thereby causing reformation of the deaminase and deaminating the base of interest in said target nucleic acid.

Also disclosed is a method for producing a small molecule inducible base editor complex in a cell for editing a target nucleic acid bound by an sgRNA, comprising introducing the expression vectors described above and a dimerization agent into said cell under conditions where said split deaminase reforms upon binding between said operably linked specific binding pair members, thereby catalyzing base editing at the site bound by said sgRNA.

Finally, kits for practicing the methods described above are also provided.

### BRIEF DESCRIPTION OF THE DRAWINGS

FIG. 1. The Base Editor Formula. Base editing involves partnership among different domain modules with segregated functions. The modules can be fused in sequence with various permutations (or approximated by binding interactions). A targeting module (TM) localizes to a particular genomic site. The tethered modifying module (MM) edits the local DNA, although it can also act at other sites upon overexpression. Skewing downstream repair pathways through accessory modules (MM_{X}) can improve efficiency. At right is shown a schematic depicting split DNA deaminases as a means to exert control over when and where base editors act.

FIG. 2A-2G (FIG. 2A) Schematic showing the topology of the DNA deaminase fold, with the active site defined by Zn-interacting residues. Selected sites targeted for insertional mutagenesis in AID* are highlighted. (FIG. 2B) Mutation frequency, as measured by the frequency of acquired rifampin resistance upon expression of AID variants in E. coli. AID(E58A), catalytically inactive control. Each individual data point is indicated (n≥3) on the log-scale plot, with mean and standard deviation shown. (FIG. 2C) A table showing GFP insertion sites tested in AID loops. (FIG. 2D) A table showing representative split sites in loop between α2-β3 DNA deaminases with structural homology to AID. (FIG. 2E) A schematic diagram of AID*-SPL2. (FIG. 2F) Co-expression of Split2 N- and C-terminal components is shown to generate a fluorescent, active deaminase complex. Specifically shown is the in vitro reconstitution when AID is split between its α2 helix and β3 strands (position 72) with a split GFP. An in vitro assay to measure deaminase activity on a labeled oligonucleotide substrate. UDG, uracil DNA glycosylase and a representative denaturing gel (100 nM DNA, 200 nM enzyme) showing unreacted substrate (C) and product (U) controls highlights that the spontaneously-assembled AID*-SPL2 is active. Product formation was also quantified as a function of enzyme concentration (n=3) and fit to a sigmoidal dose-response curve to determine the amount of enzyme needed to convert half of the substrate (EC₅₀) under these fixed reaction conditions.

FIGS. 3A-3E. Intact, inserted, and split DNA deaminase constructs with A3A. (FIG. 3A) Construct schematics for A3A and A3A-INS2 variants used to determine the impact of optGFP insertion in E. coli. (FIG. 3B) Left—an in vitro assay to measure deaminase activity on a labeled oligonucleotide substrate. UDG, uracil DNA glycosylase. Middle—a representative denaturing gel (100 nM DNA, variable enzyme concentration) is shown, along with unreacted substrate (C) and product (U). Right—product formation was quantified as a function of enzyme concentration (n=3) and fit to a sigmoidal dose-response curve to determine the amount of enzyme needed to convert half of the substrate (EC₅₀) under these fixed reaction conditions. (FIG. 3C) Construct schematics for mammalian expression of A3A-INS2, A3A(E72A)-INS2, and A3A-SPL2 variants used to determine the impact of optGFP insertion on the DNA damage response in HEK293T cells. (FIG. 3D) HEK293T cells were transfected with catalytic mutant A3A(E72A)-INS2, A3A-INS2, or co-transfected with A3A-SPL2_{N} and A3A-SPL2_{C}. After transfection, cells were stained for γH2AX and sorted for both GFP and γH2AX expression. The bar plot depicts frequency of GFP+ or GFP+/γH2AX+ cells after transfection of HEK293T cells with the indicated constructs. The mean and standard deviation from n=3 replicates is shown. (FIG. 3E) Representative immunofluorescent images of transfected U2OS cells are shown. DAPI stain highlights the nucleus, GFP staining shows expression or split complementation, and γH2AX serves as a marker of active A3A-mediated DNA damage.

FIG. 4. Mammalian cell editing efficiency assay. A cell line expression a destabilized GFP (d2GFP) is transfected by base editing variants and a sgRNA targeting gfp. The loss of GFP expression can be measured at a given timepoint by flow cytometry as a reliable read out of mutational efficiency, as confirmed by independent sequencing experiments. Under conditions where catalytically active Cas9 edits the majority of the cells to inactivate GFP, one such (non-split) base editor (a hyperactive AID variant shown) edits to inactivate GFP better than established BE3 (rate ˜5.0%, not shown). This assay setup was employed to validate the split engineered base editors (see FIG. 7).

FIG. 5. Permutations of possible split engineered base editors. Shown is one schematic that captures a split engineered base editor. The various component, the targeting modules, modifying modules, dimerizer modules and accessory modules can be varied, all employing the same scheme for splitting the deaminase. PMID numbers indicate references describing the various components depicted. Each of these disclosures are incorporated herein by reference as though set forth in full. Several exemplary regulatable specific binding pairs are shown.

FIGS. 6A-6B. Intact and split-engineered base editor constructs. (FIG. 6A) Parent construct schematics for intact BE4max scaffold editors with AID′, evoA1, and A3A. (b) Construct schematics for split-engineered seBE4max editors with AID′, evoA1, and A3A. Constructs were created by insertion of a cassette that splits the intact deaminase into two fragments, separated by a self-cleaving T2A peptide.

FIG. 7A-7C. Split-engineered base editors represent a generalizable strategy to enable small-molecule-controlled editing. (FIG. 7A) Schematics of a traditional intact base editor in the BE4max scaffold and the split-engineered base editor (seBE) strategy, including chemically induced dimerization of FRB and FKBP12 by rapamycin. (FIG. 7B) Editing efficiency can be evaluated in a HEK293T cell line containing a single copy of integrated, constitutively expressed d2gfp. The presence of d2gfp-targeting sgRNA can introduce a stop codon (Q158*) and abrogate fluorescence to generate GFP^{off} cells, which can be tracked by flow cytometry or deep-sequencing of the locus, as also depicted in FIG. 4. (FIG. 7C) At left are representative flow cytometry histograms associated with transfection of intact or seBE constructs in the presence or absence of rapamycin. At right are graphs showing the mean and standard deviation for quantification of GFP^{off} cells by flow cytometry for replicate experiments, with individual data points shown (n=3-5).

FIG. 8. Small molecule control of editing. At left—for three different base editor variants (AID′, evoA1 and A3A), the efficiency of C to T conversion at the Q158 target cytosine was quantified by deep sequencing for the intact editor or split editors with or without rapamycin. The mean and standard deviation are noted, with individual data points shown (n=3-6). Fold-change (FC) is the ratio of mean values for the higher versus the lower condition in each comparison. At right—the more complete editing footprints across the d2gfp locus for each BE, seBE, and rapamycin condition. The PAM is located at base −1 to −3, with the sgRNA protospacer from base 0 to 20. The target cytosine base within the Q158 codon is noted with a blue arrow. Data represent position-wise averages of three biological replicates.

FIG. 9. Split-engineered base editors permit efficient editing across genomic sites and tunable levels of inducible control. A graph showing target editing efficiency at seven distinct genomic loci involving epigenetic regulators. Cells were untreated or transfected with evoA1-BE4max or evoA1-seBE4max in the absence or presence of rapamycin. C or G describes whether the coding of non-coding strand cytosine is targeted, respectively, with the subscript denoting the position relative to the PAM. The mean and standard deviation for editing at the target base are noted after locus deep sequencing, with data from individual replicates shown (n=3). Right—mean value and standard deviation for editing across the seven distinct loci are plotted. The fold-charge (FC) is the ratio of mean values for the higher versus the lower condition in each comparison.

FIG. 10. sgRNA-dependent on- and off-target editing with EMX1 and FANCF targeting. HEK293T cells were untreated, transfected with evoA1-BE4max, or evoA1-seBE4max in either the absence or presence of rapamycin. For EMX1 and FANCF, the target loci and the two most common sgRNA-dependent off-target editing sites (OT1/OT2) were amplified and analyzed by deep sequencing. C or G describes whether the coding of non-coding strand cytosine is targeted, respectively, with the subscript denoting the position relative to the PAM. The mean and standard deviation for editing at the target base are noted after locus deep sequencing, with data from individual replicates shown (n=3). Complete editing footprints were also identified (data not shown). The mean values for each sgRNA-dependent off-target site are plotted at right. The fold-charge (FC) is the ratio of mean values for the higher versus the lower condition in each comparison.

FIG. 11. Split engineered base editors show low transcriptome-wide C to U mutations. Total RNA was analyzed using the RADAR pipeline (RNA-editing Analysis-pipeline to Decode All twelve-types of RNA-editing events). RNA edits that were present in the untreated (sgRNA-only) samples were removed with analysis performed only on unique editing events present in the experimental samples. At top the bar graph reports on the fraction of total edits detected that are C to U edits in RNA-seq. The fold-charge (FC) is the ratio of mean values for the higher versus the lower condition in each comparison. Below the bar graph are shown are pie charts with each category of point mutation detected with three independent replicates shown separately. At right, the mean fractions of specific edits across the three replicates are provided with the highlighted value in light blue represented in the bar graph at top.

FIG. 12. Alternative expression strategy can tune the degree of regulatory control. At the top is an alternative strategy, where the T2A self-cleaving peptide separating the two split fragments (see FIG. 6B) is instead replaced with an internal ribosome entry sequence (IRES) that leads to expression of two independently translated split protein fragments with no need for protease processing. HEK293T cells expressing a single copy of integrated d2gfp were edited using evoA1-seBE4max-IRES (see FIG. 4). At bottom left—deep sequencing results demonstrating C to T conversion efficiency of the Q158 target cytosine for seBE constructs with and without rapamycin induction. Bars indicate means and error bars indicate standard deviations of n=3 biological replicates. The fold-charge (FC) is the ratio of mean values for the higher versus the lower condition in each comparison. The dotted lines represent the mean values for the intact evoA1-BE4max and T2A evoA1-seBE4max with and without rapamycin from FIG. 8 for comparison. At right—editing footprints across the d2gfp locus for each condition. The PAM is located at base −1 to −3, with the sgRNA protospacer from base 0 to 20. The target cytosine base within the Q158 codon is noted with a blue arrow. Data represent position-wise averages of three biological replicates.

FIG. 13. Representative split engineered base editor complexes. Shown are the schematics of additional split engineered base editors in the scaffold of various base editors (BE3, BE4max, or A base editor, ABE). The constructs contain promoters for mammalian (CMV enhancer, promoter) or bacterial (T7 promoter) expression. Myc, as a tag for tracking expression. NLS, nuclear localization signal. L, linker sequences. FRB, FKBP-rapamycin binding domain of mTOR. FKBP, FK506 binding protein. nCas9, nicking version of Cas9 (D10A mutant). UGI, uracil DNA glycosylase inhibitor. T2A, self-cleaving peptide sequence. AID, activation induced deaminase. rA1, rat APOBEC1. A3A, APOBEC3A. TadA, mutant TadA domain with DNA deaminase activity. For each DNA deaminase, the domain is split into N-terminal (n) or C-terminal (c) fragments (eg. AIDn, AIDc).

FIG. 14. Strategies for the design of split, evolved base editors. Three exemplary linkage strategies for integrating a split-deaminase into different base editing designs are highlighted. The designs aim to address concerns about constitutively active enzyme, which can mutate independent of targeting by dCas9, via small-molecule control over the deaminase. The designs allow for varying degrees of temporal or spatiotemporal control over the base editors, for example with the two components approximating to one another at specific genomic locations in seBEc.

FIGS. 15A-15B. Constructs useful for the practice of the present invention. Sequences for each construct are found in SEQ ID NO: 35-58.

### DETAILED DESCRIPTION

### The Base Editing Complex.

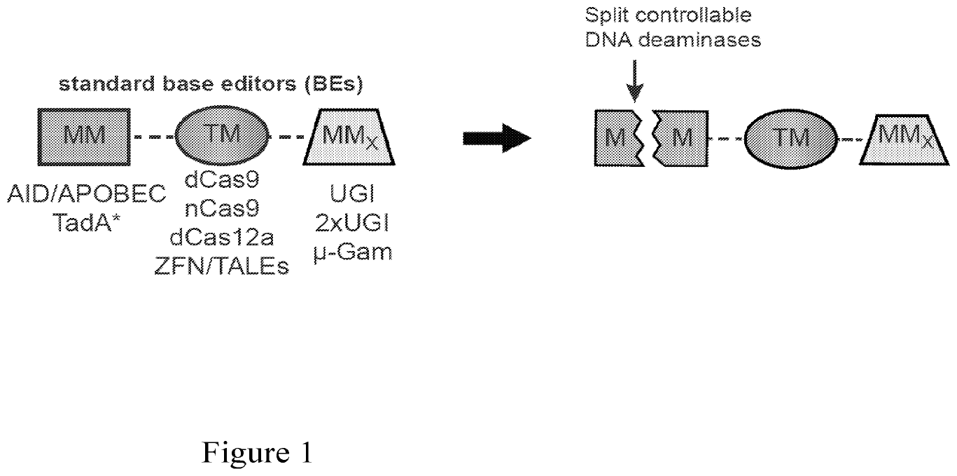

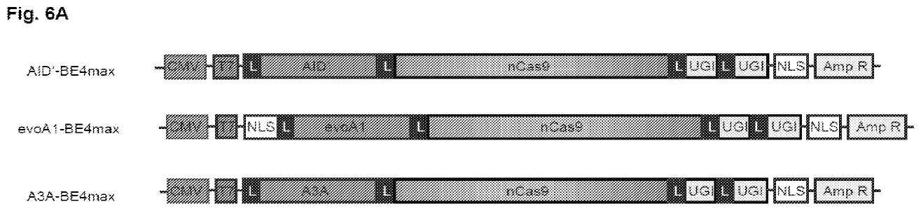

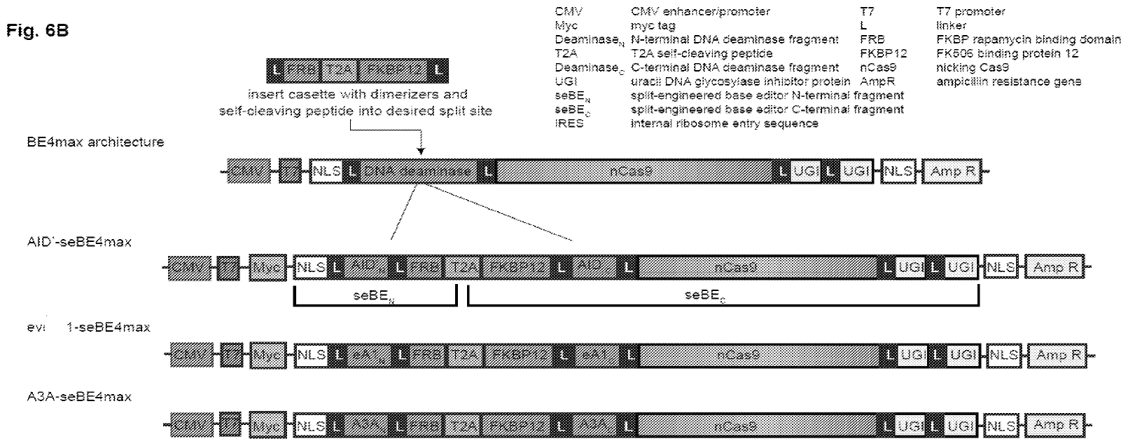

The recent repurposing of natural base editors for targeted genome editing has transformative potential (3). The typical formula for a base-editing (BE) complex (FIG. 1) involves a DNA targeting module (TM) partnered with an DNA deaminase enzyme (a modifying module, MM) and varied accessory modules (MMₓ). The initial groundbreaking base editing effort employed rat APOBEC1 as the MM, and catalytically-inactive dCas9 as the TM. Targeting of the complex was achieved via a single-guide RNA (sgRNA), which plays a dual role in localization and in the dCas9-mediated unwinding of the target site to generate single-stranded DNA, the obligate substrate of DNA deaminases. With this BE1 construct, in cis incorporation of UGI—a small phage-derived protein that potently inhibits uracil DNA glycosylase to suppress the base excision repair pathway—increases the efficiency of editing. BE2 constructs can be modified in BE3 to permit nicking (nCas9), which increases efficiency, but also promotes more insertions/deletions. The strategy of coupling to UGI has been extended to constructs with AID and the A3 enzyme APOBEC3A (A3A)(6-9). See FIG. 6, for example, where this complex is referred to as standard base editor (BE4max based) using A3A. TadA, an AID/APOBEC relative that deaminates adenosine to promote A:T to G:C changes, has also been evolved for DNA activity and employed as a MM. To date, efforts to improve base editors have largely focused on manipulating Cas9 or other TMs, testing different DNA deaminases, or altering accessory modules. Harnessing extensive existing knowledge of DNA deaminase structure and function in a rational and concerted manner has yet to be achieved, with the exception of a few precedents noted below. This represents a critical frontier, as these deaminases are naturally characterized by biochemical features, discussed next, that are important to their proper physiological function, but constrain them from achieving their full biotechnological potential.

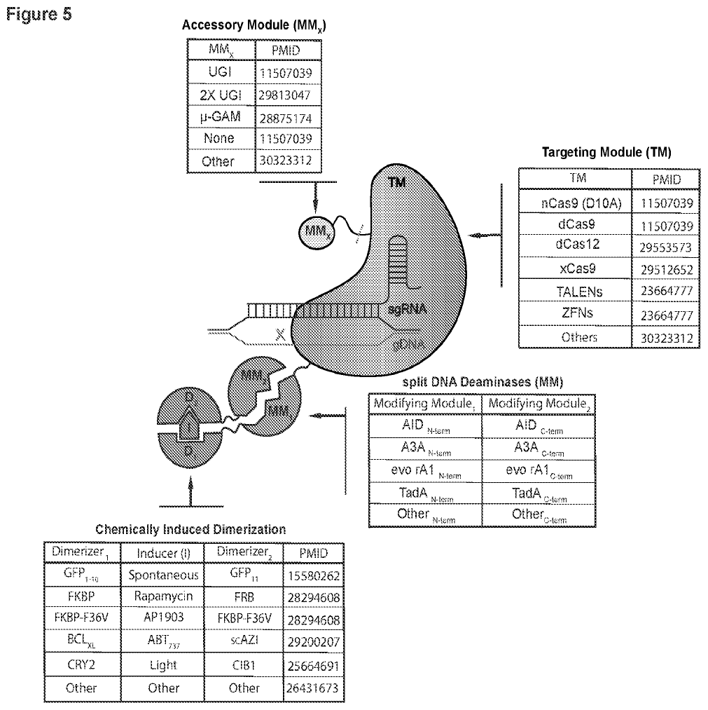

FIG. 5 lists a number of different components that can be substituted in the MM, TM and MMx modules in the editing constructs described herein.

### The Therapeutic Utility of Base Editors.

Targeted base editing has applications across biology and medicine. While CRISPR/Cas9 based approaches are effective in generating knockout by causing dsDNA breaks, these result in heterogenous knockouts given unpredictable dsDNA break repair pathways and can also promote unwanted translocations. Base editors, by contrast, have the possibility of precisely introducing stop codons (CRISPR-Stop) to knockout genes without heterogeneity (42-44). Furthermore, base editors can make precise point mutations to correct disease alleles or make neomorphic protein variants, which is not possible with Cas9 alone in the absence of homology directed repair. Base editing can therefore be used to make knockouts more precisely, to reverse targeted mutations, and to edit primary cells or hosts with less risk.

By exploiting what we know about the mechanism, structure and function of DNA deaminases, existing base editors have been transformed into more effective and therapeutically useful reagents.

### Definitions:

The terms “polynucleotide”, “nucleotide”, “nucleotide sequence”, “nucleic acid”, and “oligonucleotide” are used interchangeably in this disclosure. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “exogenous” nucleic acid can refer to a nucleic acid that is not normally or naturally found in or produced by a given bacterium, organism, or cell in nature. The term “endogenous” nucleic acid can refer to a nucleic acid that is normally found in or produced by a given bacterium, organism, or cell in nature.

The term “recombinant” is understood to mean that a particular nucleic acid (DNA or RNA) or protein is the product of various combinations of cloning, restriction, or ligation steps resulting in a construct having a structural coding or non-coding sequence distinguishable from endogenous nucleic acids found in natural systems.

The terms “construct”, “cassette”, “expression cassette”, “plasmid”, “vector”, or “expression vector” is understood to mean a recombinant nucleic acid, generally recombinant DNA, which has been generated for the purpose of the expression or propagation of a nucleotide sequence(s) of interest, or is to be used in the construction of other recombinant nucleotide sequences.

As used herein, a “modulating module” (MM) refers to the deaminase module of the base editors described herein. Exemplary MMs include for example, AID, APOBEC3 enzymes and TadA.

A “targeting module” localizes the base editing complex to the genomic region to be edited. Targeting modules can include for example, dCas9, nCas9, dCas12, ZFNs and TALENs.

An “accessory module” can optionally be included which are useful for controlling down stream repair pathways, thereby influencing efficiency of editing. Suitable accessory modules can encode a uracil glycosylase inhibitor (UGI) in one or multiple copies or μGAM for example.

The term “promoter” or “promoter polynucleotide” is understood to mean a regulatory sequence/element or control sequence/element that is capable of binding/recruiting an RNA polymerase and initiating transcription of sequence downstream or in a 3′ direction from the promoter. A promoter can be, for example, constitutively active, or always on, or inducible in which the promoter is active or inactive in the presence of an external stimulus. Example of promoters include T7 promoters or U6 promoters.

“Deamination” is the removal of an amino group from a molecule. Enzymes that catalyze this reaction are called deaminases. Deaminases include, without limitation, APOBEC1, APOBEC3A, APOBEC3B, APOBEC3C, APOBEC3DE, APOBEC3F, APOBEC3G, Activation-induced cytidine deaminase (AID), CDA from lamprey, Adenosine Deaminases acting on tRNA (TadA), and Adenosine Deaminase acting on dsRNA (ADAR). More broadly this deaminase family includes homologs from various species all of which are thought to catalyze similar reactions on nucleic acids as described in Krishnan et al. (Proc Natl Acad Sci USA. 2018; 115(14):E3201-E3210 and Iyer et al. (Nucleic Acids Res. 2011 December; 39(22):9473-97).

An “adapter or adaptor”, or a “linker” for use in the compositions and methods described herein is a short, chemically synthesized, single-stranded or double-stranded oligonucleotide that can be ligated to the ends of other DNA or RNA molecules. Double stranded adapters can be synthesized to have blunt ends to both terminals or to have sticky end at one end and blunt end at the other, or sticky ends at both ends. For instance, a double stranded DNA adapter can be used to link the ends of two other DNA molecules (i.e., ends that do not have “sticky ends”, that is complementary protruding single strands by themselves). It may be used to add sticky ends to cDNA allowing it to be ligated into the plasmid much more efficiently. Two adapters could base pair to each other to form dimers. A conversion adapter is used to join a DNA insert cut with one restriction enzyme, say EcoRl, with a vector opened with another enzyme, Bam Hl. This adapter can be used to convert the cohesive end produced by Bam Hl to one produced by Eco Rl or vice versa. One of its applications is ligating cDNA into a plasmid or other vectors instead of using Terminal Deoxynucleotide Transferase enzyme to add poly A to the cDNA fragment.

Alternatively, the linker may be a peptide linker such as those that occur between protein domains. Short peptide linkers are often composed of flexible residues like glycine and serine so that the adjacent protein domains are free to move relative to one another. Exemplary linkers include without limitation, 2 amino acid GS linkers, 6 amino acid (GS)x linker, 10 amino acid (GS)x linker, short linkers (Gly-Gly-Ser-Gly; SEQ ID NO: 1), Middle linkers (Gly-Gly-Ser-Gly; SEQ ID NO: 1) x2 and long linkers (Gly-Gly-Ser-Gly; SEQ ID NO: 1) x3, flexible linkers 2x(GGGS; SEQ ID NO: 2), 2x (GGGGS(SEQ ID NO: 3) and 13 amino acid linkers (GGGS GGGGS GGGS; SEQ ID NO:4).

The term “operably linked” can mean the positioning of components in a relationship which permits them to function in their intended manner. For example, a promoter can be linked to a polynucleotide sequence to induce transcription of the polynucleotide sequence.

The terms “sequence identity” or “identity” refers to a specified percentage of residues in two nucleic acid or amino acid sequences that are identical when aligned for maximum correspondence over a specified comparison window, as measured by sequence comparison algorithms or by visual inspection. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity.

The term “comparison window” refers to a segment of at least about 20 contiguous positions in which a sequence may be compared to a reference sequence of the same number of contiguous positions after the two sequences are aligned optimally. In a refinement, the comparison window is from 15 to 30 contiguous positions in which a sequence may be compared to a reference sequence of the same number of contiguous positions after the two sequences are aligned optimally. In another refinement, the comparison window is usually from about 50 to about 200 contiguous positions in which a sequence may be compared to a reference sequence of the same number of contiguous positions after the two sequences are aligned optimally.

The terms “complementarity” or “complement” refer to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 4, 5, and 6 out of 6 being 66.67%, 83.33%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 40%, 50%, 60%, 62.5%, 70%, 75%, 80%, 85%, 90%, 95%, 97%, 98%, 99%, or 100%, or percentages in between over a region of 4, 5, 6, 7, and 8 nucleotides, or refers to two nucleic acids that hybridize under stringent conditions.

In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence”. In aspects of the invention, an exogenous template polynucleotide may be referred to as an editing template. In an aspect of the invention the recombination is homologous recombination.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net).

A “zinc finger nuclease” as used herein refers to artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms.

“Transcription activator-like effector nucleases” (TALEN) are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to practically any desired DNA sequence, so when combined with a nuclease, DNA can be cut at specific locations. The restriction enzymes can be introduced into cells, for use in gene editing or for genome editing in situ, a technique known as genome editing with engineered nucleases. Alongside zinc finger nucleases and CRISPR/Cas9, TALENs are also suitable for use in the base editing complexes of the invention.

Several aspects of the invention relate to vector systems comprising one or more vectors, or vectors as such. Vectors can be designed for expression of editing complexes of the invention (e.g. nucleic acid transcripts, proteins, or enzymes) in prokaryotic or eukaryotic cells. For example, base editing transcripts can be expressed in bacterial cells such as Escherichia coli, insect cells (using baculovirus expression vectors), yeast cells, or mammalian cells. Suitable host cells are discussed further in Goeddel, Gene Expression Technology: Methods in Enzymology 185, Academic Press. San Diego, Calif. (1990). Alternatively, the recombinant expression vector can be transcribed and translated in vitro, for example using T7 promoter regulatory sequences and T7 polymerase.

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., Molecular Cloning: A Laboratory Manual, 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid encoding the base editing complex preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter, U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

In some aspects, the invention provides methods comprising delivering one or more polynucleotides, such as or one or more vectors as described herein (e.g., encoding all or portions of the base editing complexes discussed below), one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a CRISPR enzyme in combination with (and optionally complexed with) a guide sequence, a zinc finger nuclease or a TALEn is delivered to a cell. Conventional viral and non-viral based gene transfer methods can be used to introduce nucleic acids in mammalian cells or target tissues. Such methods can be used to administer nucleic acids encoding components of a base editing system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line.

In one aspect, the invention provides for methods of modifying a target polynucleotide in a eukaryotic cell, which may be in vivo, ex vivo or in vitro. In some embodiments, the method comprises sampling a cell or population of cells from a human or non-human animal, and modifying the cell or cells. Culturing may occur at any stage ex vivo. The cell or cells may be re-introduced into the human or non-human animal.

In other embodiment, proteins comprising the base editing complex can be delivery directly into cells via use of nanoparticles, RNPs and other methods known to the skilled artisan.

### Split Deaminase Generation and Methods of Use Thereof for Controlled and Efficient Base Editing

DNA deaminases serve important roles in immune defense and other processes. Exemplary AID/APOBEC enzymes are immune enzymes. AID plays a role in somatic hypermutation, the mechanism by which antibody encoding genes are mutated and affinity matured. The related APOBEC3 enzymes are also known to target retroviruses for deamination.

As mentioned above, a family of deaminases exists and includes adenosine deaminase enzymes like TadA, which catalyzes A to I mutation in tRNAs, and whose mutant variants can act on DNA rather than RNA. Notably, each of these DNA deaminases possess comparable secondary structures facilitating identification of suitable splitting sites which can be effectively reassembled when tagged with proteins or agents having specific binding affinity for one another which spontaneously reassemble when in proximity. Strategies for splitting DNA deaminase based on secondary structure within “families of deaminases” are described herein.

The ability to precisely edit specific bases has broad biotechnological potential in many practical and therapeutic approaches. While base editing of the human genome is the most exciting and promising of these approaches, many other applications exist, for example in modification of epigenetic sequences, agriculture and the biofuel industry. Other intriguing applications include directed somatic hypermutation for generation of improved antibodies and other therapeutic proteins.

In order to more precisely control and target base editing, the split DNA deaminases described herein are constructed such that reassembly is effected by the binding of a small molecule to an added domain that induces split deaminases to spontaneously reassemble, thereby reforming the split enzyme into an active and efficient deaminase. This inventive approach enables simultaneous spatiotemporal and small molecule control over activation of the mutator enzyme conferring a number of advantages including introduction of mutations at a precise time and location which has the benefit of decreasing off target, undesired activities or delaying the introduction of mutations until a time when it is desirable.

Our strategy entailed first identifying a control point for insertion of the spontaneously reassembling binding partners, then splitting the enzyme into demonstrably inactive parts which can effectively and spontaneously reassemble when tagged with proteins that spontaneously come together, finally providing an inducer element which alters the protein partners from ones which spontaneously reassemble to those that come together only in the presence of an inducing agent, and demonstrating that small-molecule inducible, precise base editing has been achieved.

The secondary structure of the DNA deaminase fold was examined to identify “control points” or insertion sites for small regulatory elements which would allow for small-molecule control over the deaminase reassembly and activity.

In initial studies, a foreign protein/domain was inserted into an enhanced hyperactive version of AID described in U.S. patent application Ser. No. 16/025,261 (See for example, SEQ ID NO: 20 of the '261 patent application) which is incorporated herein by reference as though set forth in full. This mutant version of the human DNA deaminase AID, involved in antibody maturation was assessed and the loop regions which appeared to be tolerant to insertion of control elements (e.g., Green Fluorescent Protein GFP fragments) were identified as described hereinbelow. Several candidate locations for insertion were identified. We used an E. coli-based rifampin mutagenesis assay to evaluate activity (See Kohli et al., J Biol Chem. (2009) 284:pages 22898-904). In this assay we measure the activity level of a mutator by measuring how many E. coli can be turned resistant to the antibiotic rifampin when the enzyme is turned on.

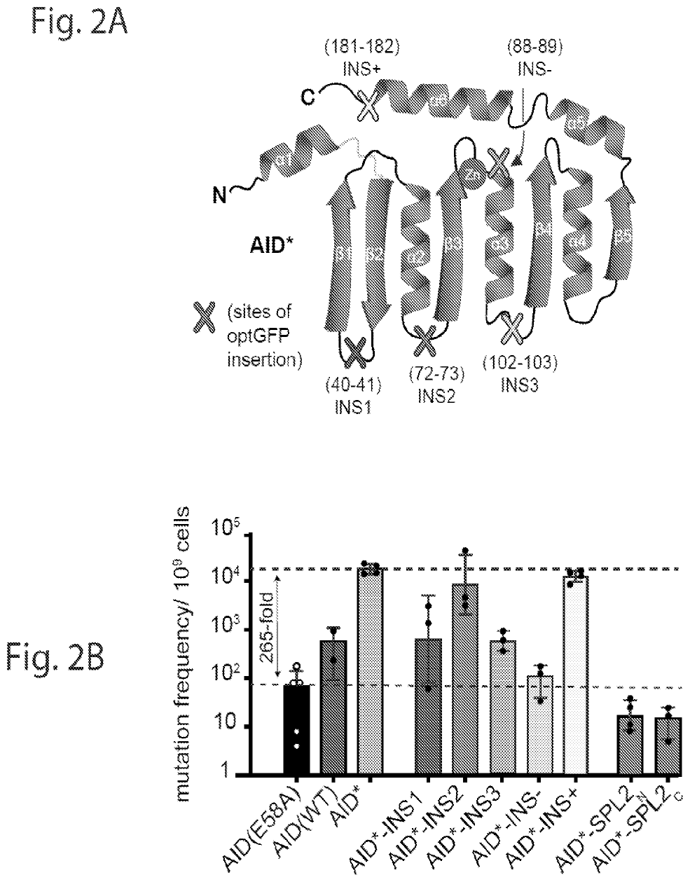

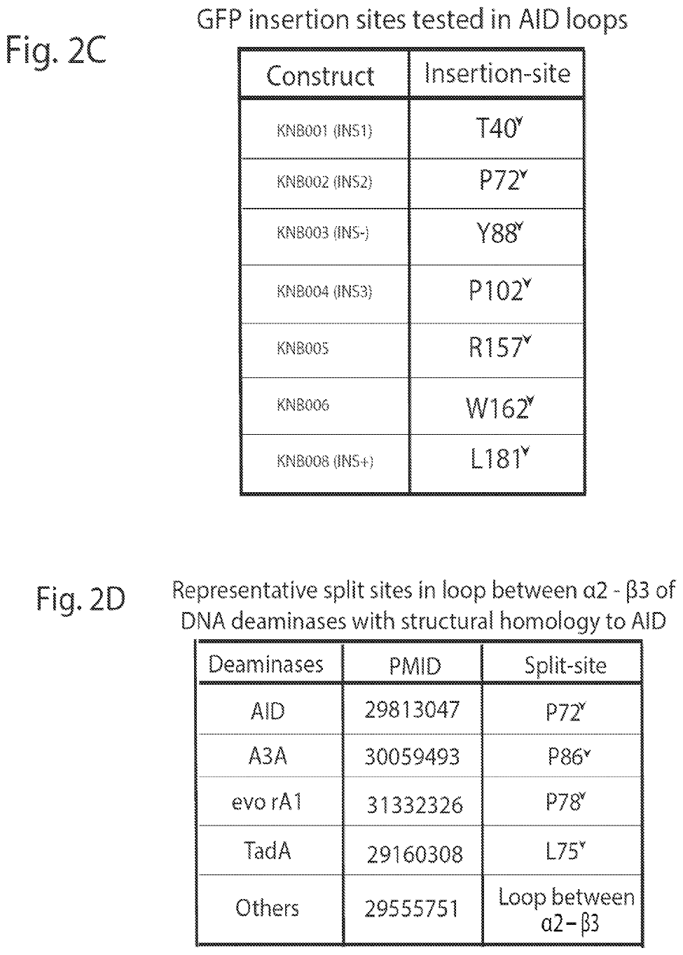

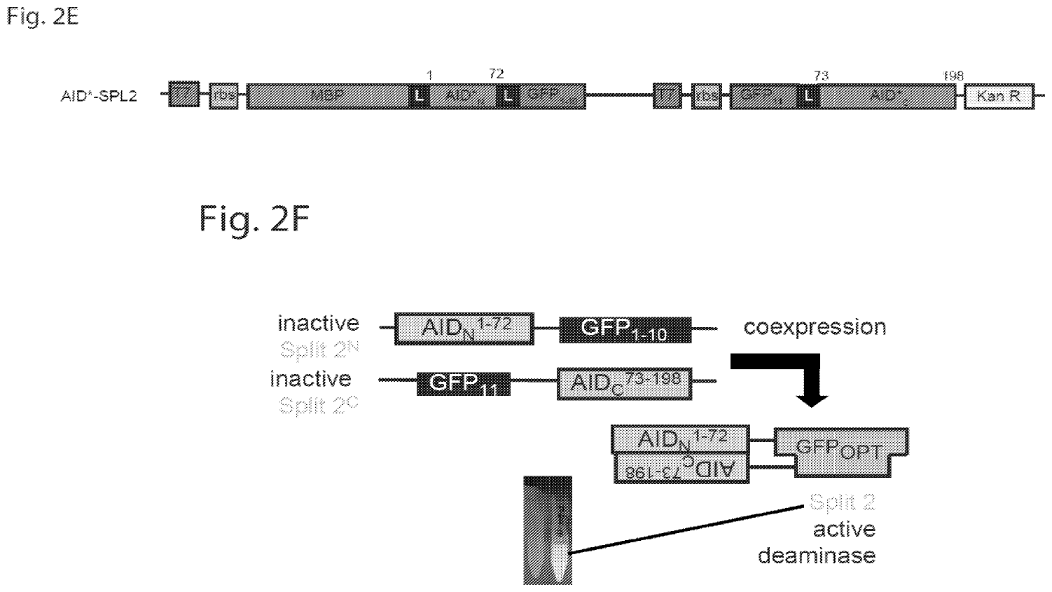

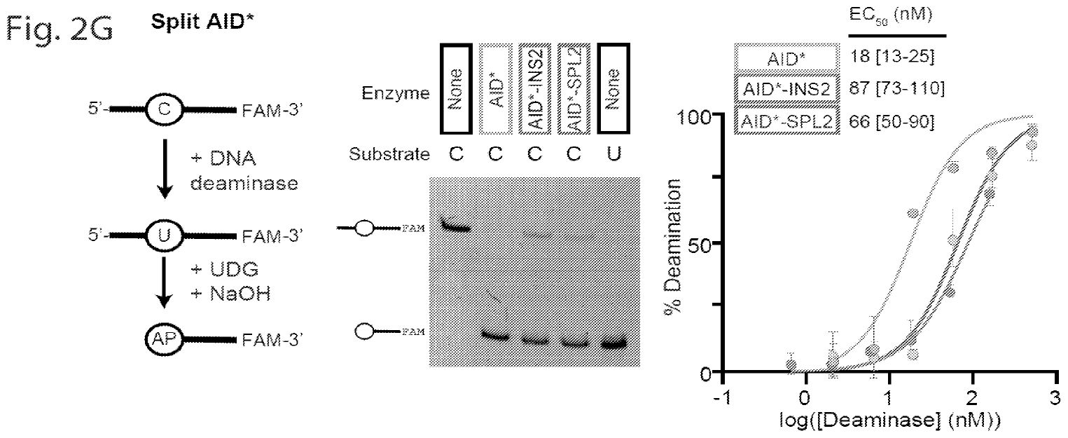

This study led us to focus on inserting into the loop between alpha2 and beta3, but other candidate sites are also suitable as shown in FIG. 2.

Having identified candidate sites for insertion, we then assessed whether the protein could be split into two inactive components. We split the GFP between beta stand 10 and 11, which is known as split GFP, and resulted in splitting of AID into N- and C-terminal halves. We showed that the split enzymes are inactive by themselves in the rifampin based resistance assay (see FIG. 2). If we express them together, the split GFP can spontaneously reassemble and reconstitute a functional AID/APOBEC enzyme that is active in vitro. See FIGS. 2F and 2G.

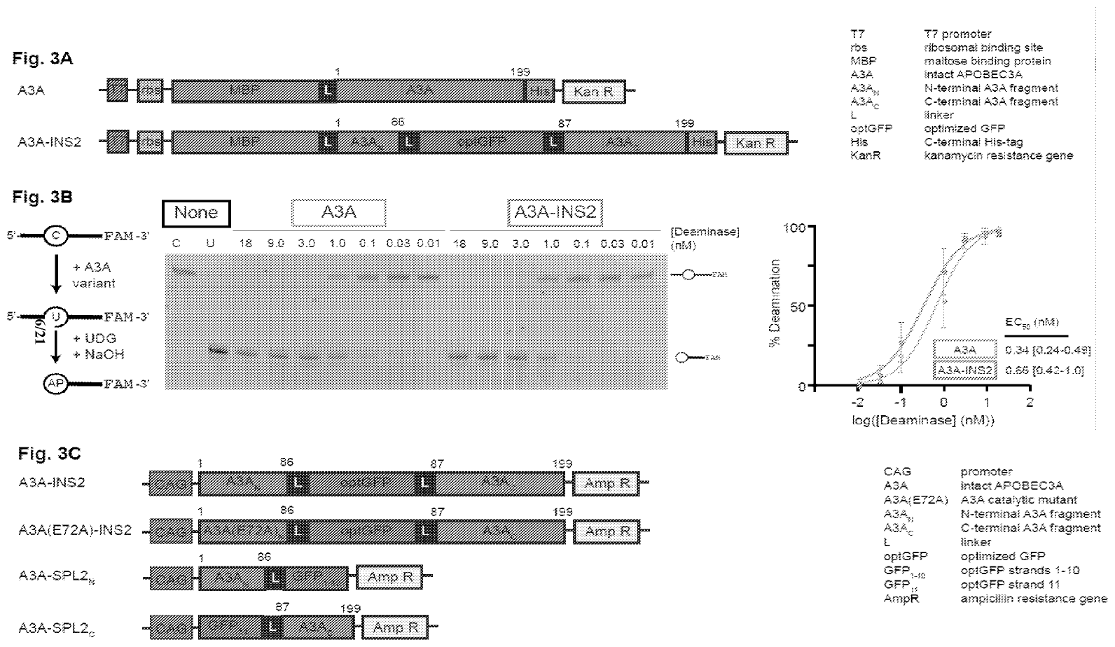

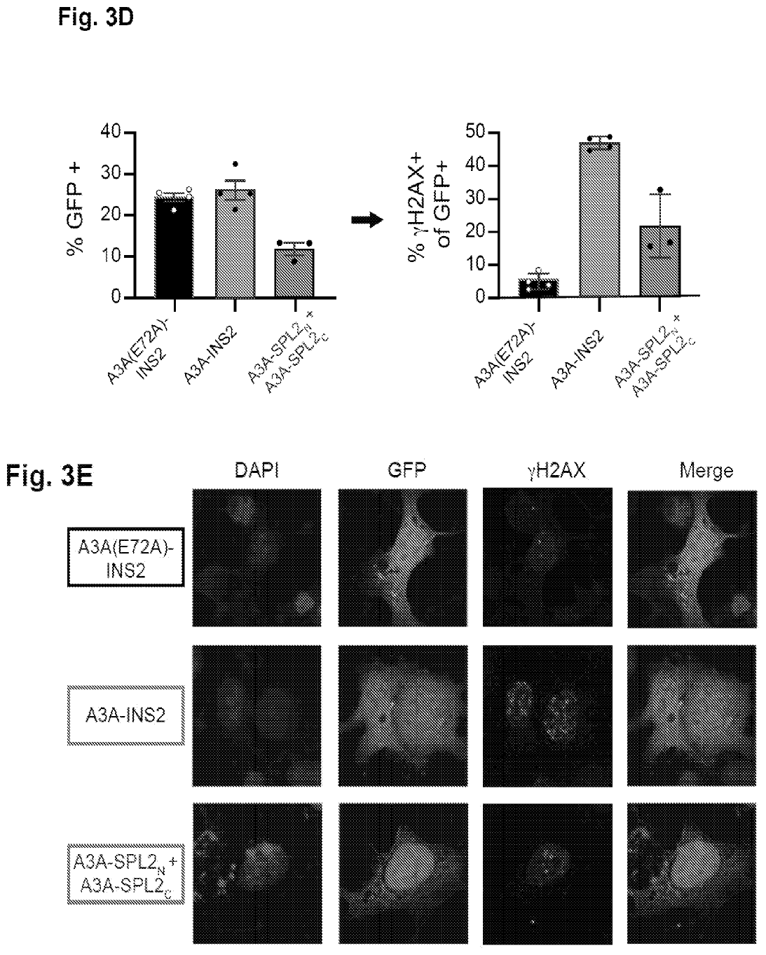

The analogous approach with other AID/APOBEC family members has also been assessed as described herein, including APOBEC3A. See FIG. 2D for a list of split sites in these other AID/APOBEC family members. These other family members when split, are also amenable to reassembly using small molecule binding partners. We validated that the split site most active for AID also allows for splitting of APOBEC3A (see FIG. 3A-B).

Using this split A3A, we also tested the system employing the spontaneously reassembling split deaminase in mammalian cell lines (HEK293 and HeLa cells). When we express two inactive APOBEC3A splits together, GFP spontaneously reassembles and we see DNA damage to the mammalian genome, as measured by a DNA damage marker. See FIG. 3C-3E. The focus of this analysis was the DNA deaminase domain by itself.

Having established the split sites and the feasibility of spontaneous reassembly with split GF, the last steps of tool development for split base editor development was switching from split, spontaneously reassembling GFP to two proteins which can reassemble under small molecule control, and moving from the DNA deaminase domain by itself to a more complex scaffold of a base editor complex. Here, the dimerization domain is exemplified by FKBP-FRB, which can be brought together with rapamycin, and use of the the Cas9-based base editor platform. Other small molecules for this purpose, include, without limitation those shown in FIG. 5.

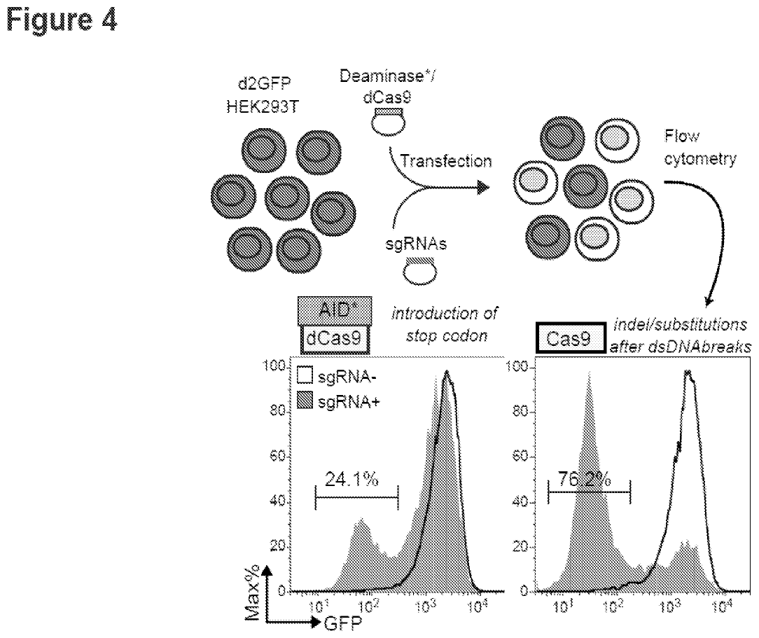

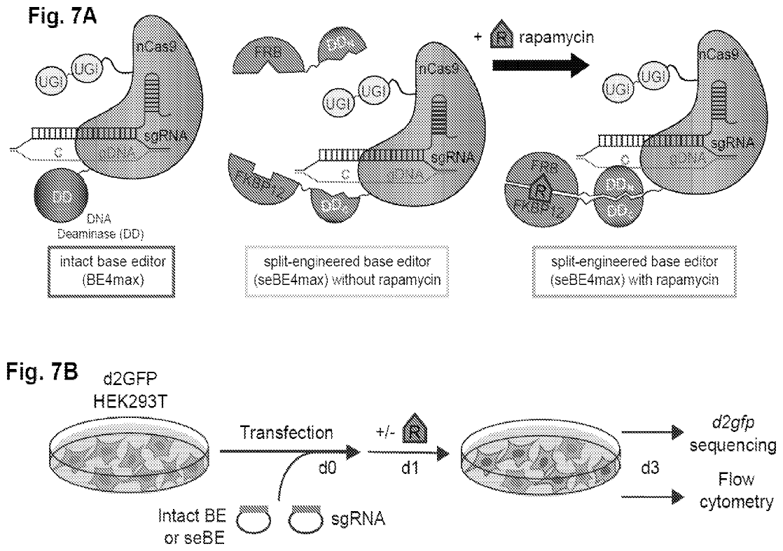

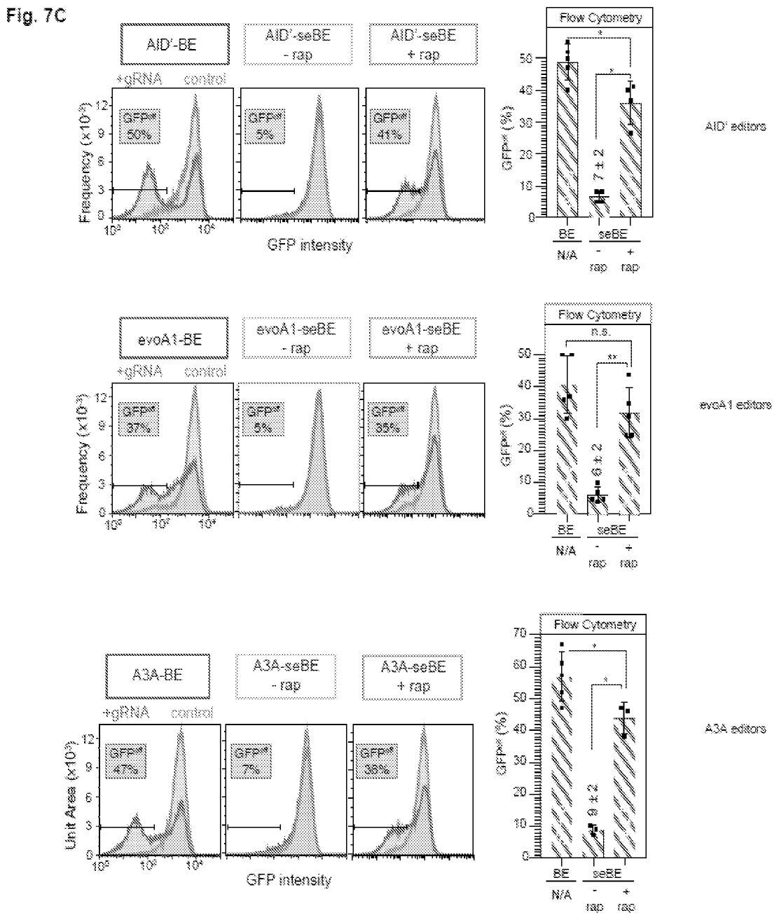

Using the seBEa scaffold with split AID, A3A, or evolved APOBEC1 (evoA1) we have achieved the goal of small molecule control over base editing. Using an assay measuring inactivation of a single copy of GFP in cell lines (See FIG. 4), base editing does not occur in the absence of the small molecule rapamycin, but can be specifically and efficiently induced by rapamycin and the correct targeting guide RNA (See FIGS. 7-10). We anticipate that using the system described off target mutations will be reduced and editing can be turned “off” via removal of the dimerization agent, in this example, rapamycin.

Now that identified suitable sites for splitting deaminases, these can be substituted in the editing constructs described herein. Notably, other DNA deaminases can be split at analogous sites between alpha2 and beta3. Existing base editors constructs can be altered in split engineered base editors by the insertion of a DNA cassette into at the split site, as schematized in FIG. 6. See the sequences provided hereinbelow.

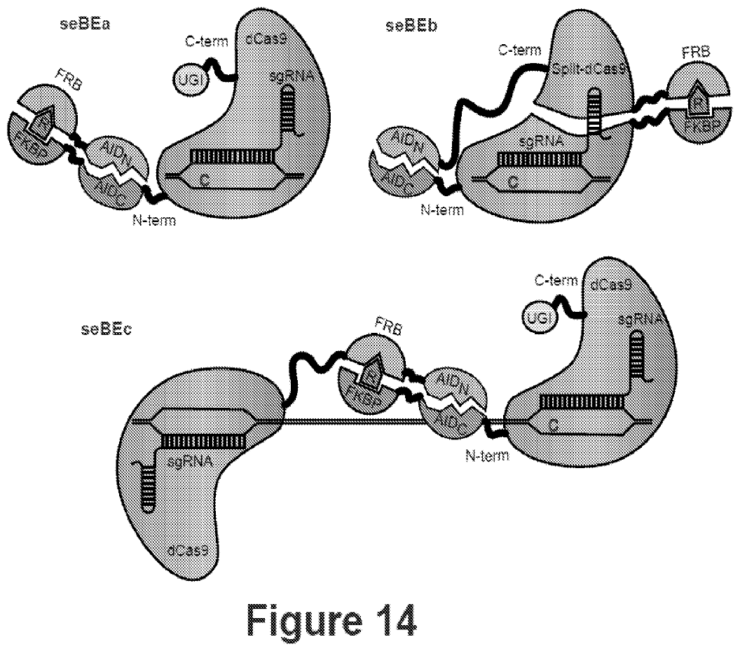

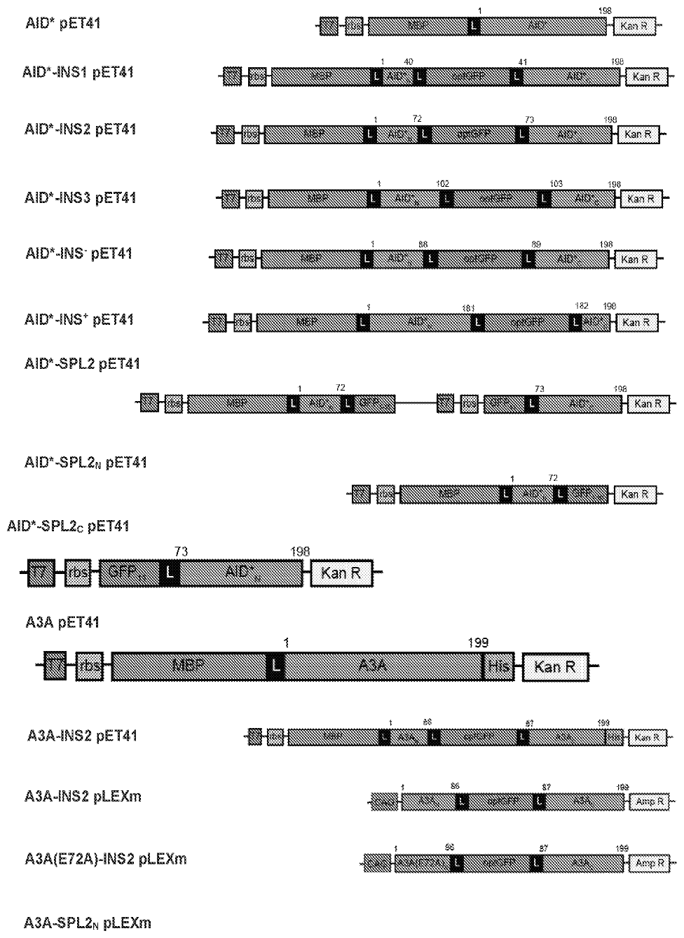

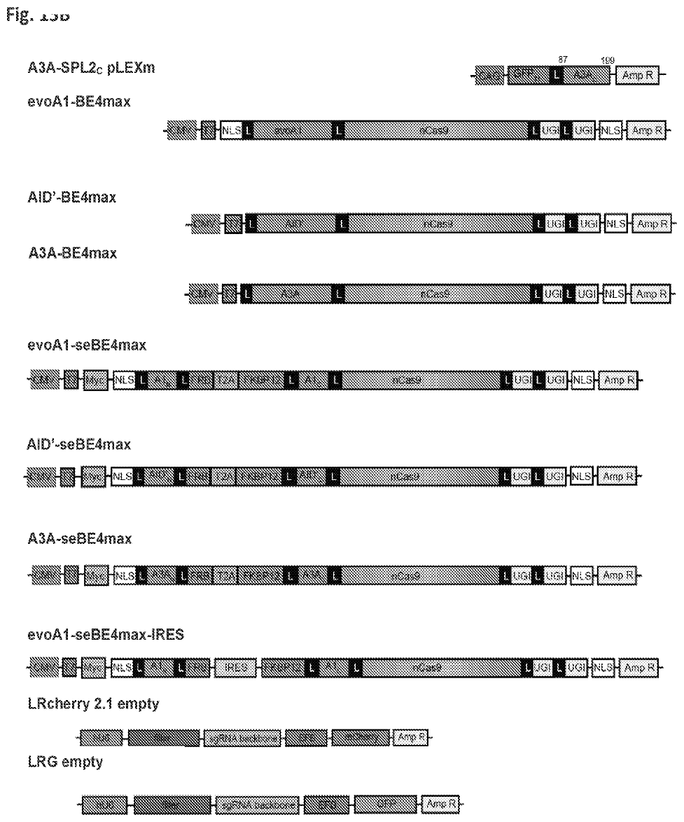

We envision various combinations of the DNA deaminase with different Targeting Modules beyond nCas9, in different orders (e.g. Cas9-deaminase, instead of Deaminase-Cas9, etc), and with various accessory modules. Each of these could be joined with linkers of various lengths or make-up. See FIGS. 13 and 14. Moreover, now that “control point” has been identified, we can use alternatives to the FKBP/FRB/rapamycin system. Other small-molecule induced dimerizers can be inserted at the control point instead.

The following methods are provided to facilitate the practice of the present invention.

### Cell Culture

HEK293T d2GFP contains a single integrated copy of destabilized GFP in its genome. The cell line was maintained in Dulbecco's Modified Eagle's Medium with L-Glutamine, 4.5 g/L Glucose and Sodium Pyruvate (Corning) supplemented with 10% (v/v) bovine calf serum (CS) and 1% (v/v) Penicillin-Streptomycin mix, at 37° C. with 5% CO₂.

### Design and Cloning of Intact and Split Base Editor Constructs

For mammalian base editing constructs, the intact or split-engineered constructs were cloned into the scaffold of pCMV_BE4max (Addgene Plasmid #112093), which contains rat APOBEC1. The parent plasmid contains a NotI restriction site. An additional XmaI restriction site was added into pCMV_BE4max using the Q5 Site-Directed Mutagenesis Kit (NEB) to facilitate cloning. The deaminase sequences were amplified from their respective pET41 plasmids, introducing a region of overlap. AID′ differs from AID* in that it contains a smaller subset of mutations, including K10E, T82I, D118A, R119G, K120R, A121R, and E156G. To facilitate cloning of seBE constructs, gene fragments were synthesized (IDT) containing Deaminase_{N}-FRB, the T2A self-cleaving peptide between the two fragments, and FKBP12-Deaminase_{C}. The associated strategy for linkers between domains was derived from that recently employed to split human TET2⁴⁷. Using the gene fragments, all BE4max and seBE plasmids were then constructed using Gibson Assembly Master Mix (NEB), merging the relevant gene fragments with the NotI/XmaI digested vector. Notably the intact AID′-BE4max and A3A-BE4max lack the N-terminal NLS present in BE4max vectors. A3A-seBE contains a missense mutation (M13I) as a result of a PCR error, which does not appear to impact activity.

The evoA1-seBE4max-IRES construct, where the two split protein fragments are independently translated, was cloned into the scaffold of evoA1-seBE4max. The IRES sequence fragment was amplified from Addgene Plasmid #105594⁴⁸ with Phusion High-Fidelity DNA Polymerase (NEB). The vector backbone of evoA1-seBE4max was amplified, excluding the T2A sequence. The vector and IRES sequence fragment were then joined using the In-Fusion HD Cloning system (TBUSA).

The sgRNA expression plasmids were constructed using oligonucleotide cassettes for cloning. Briefly, the primers listed in the Supplementary Information were annealed and phosphorylated using T4 Polynucleotide Kinase (NEB) according to the manufacturer's instructions and further purified using the oligo clean and concentrator kit (Zymo Research). Next, LRcherry2.1 plasmid⁴⁹ or LRG plasmid (Addgene #65656) were incubated with restriction enzyme Esp3I (Thermo Fisher Scientific) at 37° C. for 2 hours to remove a short filler sequence, and further agarose gel purified. The sgRNA cassettes were then ligated in place of the filler using T4 DNA ligase (NEB).

### Bacterial DNA Deaminase Rifampicin Mutagenesis Assay

The mutation frequency of various DNA deaminases, including insertion constructs, were determined using a modified version of previously reported rifampin mutagenesis assay (Kohli, JBC 2009). Plasmids encoding the deaminase variant were transformed into BL21(DE3) E. coli, that already harbor a plasmid encoding uracil DNA glycosylase inhibitor (UGI) on a pETcoco2 plasmid. Overnight cultures grown in LB with kanamycin (30 ng/mL) and chloramphenicol (25 ng/mL) from single colonies were diluted to an A₆₀₀ of 0.2 and grown for 1 hr at 37° C. before inducing deaminase expression with 1 mM isopropyl β-D-1-thiogalactopyranoside (IPTG). After 4 hrs of additional growth, aliquots of cultures were separately plated on Luria Bertani (LB) agar plates containing rifampicin (100 μg/mL) and plasmid-selective antibiotics. The mutation frequencies were then calculated by the ratio of rifampicin resistant colonies to total population. For bacterial work with AID*, the parent pET41 plasmid with AID* combines three different sets of previously described²⁹⁻³¹ mutations that increase activity or solubility (K10E, F42E, T82I, D118A, R119G, K120R, A121R, H130A, R131E, F141Y, F145E, and E156G) in a construct with an N-terminal maltose binding protein tag (MBP). The plasmids named AID*-INS contain an insertion of optGFP flanked by linkers at each position within a specified loop of AID*. The N-terminal fragment of AID (AID*_{N}) and C-terminal fragment of AID (AID*_{C}) were generated by PCR amplification from the AID* parent plasmid with primers listed in Supplementary Table 2. A sequence containing linker-optGFP-linker was obtained as a gene fragment (Integrated DNA Technologies, IDT) and amplified with primers provided below, which add flanking regions that permit overlap extension PCR. Overlap extension PCR was performed to fuse the three fragments encoding AID*_{N}, linker-optGFP-linker, and the AID*_{C}, using 10 cycles of amplification without primers to permit fusion of fragments, followed by amplification of the entire AID*_{N}-optGFP-AID*_{C} sequence with the outer primers. PCR products from the overlap extension PCR were TA cloned (Invitrogen). Sequence-confirmed inserts were then digested with SalI and AvrII and ligated into the digested parent plasmid with T4 DNA ligase (NEB). The control plasmids containing unmutated AID (AID-WT) or its catalytically inactive analog, AID(E58A), were previously reported³⁰.

For bacterial work with split AID*, AID*-SPL2_{N} and AID*-SPL2_{C} were created using AID*-INS2 as a scaffold in the pET41 backbone. To create AID*-SPL2_{N}, the parent plasmid (AID*-INS2) was digested with KpnI and AvrII to remove the C-terminal region of AID*. Then, an oligonucleotide cassette containing a stop codon (TAG) was ligated into the digested vector. To create AID*-SPL2_{C}, the parent plasmid (AID*-INS2) was digested with XbaI and KpnI to remove AID*-SPL2_{N}. Then, a cassette containing a start codon (ATG) was ligated into the digested vector. The AID*-SPL2 plasmid, co-expressing the N-terminal and C-terminal fragments, from separate promoters was created using AID*-INS2 as a scaffold. A gene fragment was synthesized containing the C-terminal region of AID*-SPL2_{N}, the transcriptional terminator, T7 RNA polymerase promoter and N-terminal region of AID*-SPL2_{C}. This fragment was ligated into a KpnI/AvrII digested AID*-INS2 parent vector.

For bacterial expression of A3A constructs with insertion of optGFP, cloning was performed in_the scaffold of MBP-A3A-His-pET41 backbone^{45, 46} (Addgene #109231) using restriction enzymes EagI and AvrII. The appropriate optGFP-containing insert was synthesized as a gene fragment (IDT), digested with EagI/AvrII (NEB), and ligated into the similarly digested parent plasmid.

For mammalian expression of A3A constructs, plasmids were cloned into a pLEXm backbone._A3A-INS2,_A3A-SPL2_{N}, and A3A-SPL2_{C} were amplified from the pET41 construct, adding flanking regions of overlap with the pLEXm plasmid backbone. The final plasmids were then constructed using Gibson Assembly Master Mix (NEB), merging the amplified gene fragments with the EcoRI/XhoI (NEB) digested parent vector. The catalytically inactive variant A3A(E72A)-INS2 was created using Q5 Site-Directed Mutagenesis Kit (NEB).

### In Vitro DNA Deaminase Oligonucleotide Assay

For in vitro assays, purified intact, optGFP-inserted, or split DNA deaminases were expressed in BL21(DE3) cells that co-express the Trigger Factor (TF) chaperone, as previously described³³. Briefly, 600 mL cultures were grown to an OD₆₀₀ of 0.6 at 37° C. Cultures were shifted to 16° C. for 16 hours after induction with 1 mM IPTG. For AID variants, the pelleted cells were resuspended in 50 mM Tris-Cl (pH 7.5) 150 mM NaCl, 10% glycerol (wash buffer) and lysed through sonication. The soluble fraction was filtered after high-speed centrifugation and incubated with 3 mL of Amylose Resin (NEB) for 1 hr at 4° C. The resin was washed extensively prior to elution with wash buffer plus 10 mM maltose. Total protein was quantified by comparison to a BSA standard curve. For A3A variants, the pelleted cells were resuspended in 50 mM Tris-Cl (pH 7.5) 150 mM NaCl, 10% glycerol, 25 mM imidazole (wash buffer) and lysed through sonication. The soluble fraction was filtered after high-speed centrifugation and incubated with 3 mL of HisPur cobalt resin (Thermo) for 1 hr at 4° C. The resin was washed extensively prior to elution with wash buffer with 150 mM imidazole.

For the in vitro assay, a fluorescein (FAM)-labeled oligonucleotide substrate was used containing a single cytosine, along with a product control oligonucleotide containing uracil at the same location. For AID variants, the oligonucleotide substrate was co-incubated with 3-fold dilutions of the purified AID variant (520 nM to 0.6 nM) and 25 U of uracil DNA glycosylase (NEB). The reaction was performed in 20 mM Tris-HCl (pH 8.0), 1 mM DTT and 1 mM EDTA at 37° C. for 1 hr. For A3A, the oligonucleotide substrate was co-incubated with 3-fold dilutions of the purified A3A variant (18 nM to 10 pM) and 25 U of uracil DNA glycosylase. The reaction was performed in 350 mM succinic acid, sodium dihydrogen phosphate, and glycine (SPG) buffer (pH 5.5) and 0.1% Tween-20 at 37° C. for 30 min. Deamination reactions were terminated by incubation at 95° C. for 10 min. The samples were heat denatured by using 2× bromophenol blue loading dye containing 0.6 M NaOH to cleave abasic sites and 0.03 M EDTA. Samples were run on a preheated 20% acrylamide/Tris-Borate-EDTA(TBE)/urea gel at 50° C., and imaged using FAM filters on a Typhoon imager (GE Healthcare). Product formation was quantified using ImageJ by taking the ratio of substrate to product under each condition. Product formation as a function of enzyme concentration was fit to a sigmoidal dose-response curve and used to determine the EC₅₀, defined as the amount of enzyme that converts 50% of the substrate to product under the fixed reaction conditions.

### A3A Assay for DNA Damage in Mammalian Cells

HEK293T cells were transiently transfected with A3A-INS2, A3A(E72A)-INS2 or co-transfected with A3A-SPL2_{N} and A3A-SPL2_{C} constructs for 24 hours prior to incubation with γH2AX antibody (BD Pharmigen, 647) and flow cytometry analysis. Cells were gated on FITC and APC using the Fortessa Flow Cytometer (BD Biosciences), and results were analyzed using FlowJo. Statistical analysis was performed using GraphPad Prism. U2OS cells plated on coverslips were transiently transfected with A3A-INS, A3A(E72A)-INS2 or co-transfected with A3A-SPL2_{N} A3A-SPL2_{N} constructs for 24 hours prior to incubation with γH2AX antibody (Millipore Sigma) and immunofluorescent staining with Alexa Fluor 568 (Invitrogen) and DAPI. Stained cells were imaged with a Nikon MR confocal microscope and analyzed using Image J. HEK293T and U2OS cells were cultured in Dulbecco's Modified Eagle Medium (Gibco) media supplemented with 10% fetal bovine serum and 1% penicillin/streptomycin.

Base Editing Assay Using d2GFP Inactivation by Flow Cytometry

HEK293T cells were lentivirally-transduced with a constitutively expressed destabilized GFP (d2GFP) reporter (derived from Addgene #14760) and selected for individual clones that contained a single copy of integrated d2gfp. The cell line was maintained in Dulbecco's Modified Eagle Medium with L-glutamine, 4.5 g/L glucose and sodium pyruvate (Corning) supplemented with 10% (v/v) bovine calf serum (CS) and 1% (v/v) penicillin-streptomycin mix, at 37° C. with 5% CO₂. The HEK293T d2GFP cells were seeded on 24-well plates and transfected at approximately 60% confluency. 660 ng of intact BE4max or seBE4max constructs and 330 ng of LRcherry2.1 sgRNA expression plasmids were transfected using 1.5 μL of Lipofectamine 2000 CD (Invitrogen) per well according to manufacturer's protocol. Negative control samples include LRcherry2.1 plasmid lacking a protospacer (labeled as no sgRNA samples). The d2gfp-targeting sgRNA exposes a window where base editing can result in the introduction of a Q158X nonsense mutation in d2gfp. For seBE experiments, 24 hrs after transfection, rapamycin (Research Products International) was added to select wells at a final concentration of 200 nM. Transfected cells were harvested at day 3 after transfection, ensuring single-cell suspension. The percentage of d2GFP-negative and mCherry-positive (sgRNA+) cells was determined by flow cytometry with Guava Easycyte 10HT instrument (Millipore). Flow cytometry analysis was performed using FlowJo Software Version 10.7.1 (FloJo, LCC).

Genomic DNA was also collected from cells using the DNeasy Blood & Tissue Kit (Qiagen) according to manufacturer's instructions for amplification across the d2gfp locus and deep sequencing as described below. Total RNA was isolated using Direct-zol™ RNA Miniprep Plus kit (Zymo Research #R2072) following the manufacturer's protocol for sequencing as described below. For RNA-seq analysis, negative control transfections included d2gfp-targeting LRcherry2.1 plasmid without any base editor construct.

### Base Editing of Various Genomic Loci

For editing of diverse genomic loci, HEK293T cells (lacking the single copy d2gfp) were used and maintained as above. The transfection protocol was performed as described above, with the exception that different sgRNAs were used to targeting of other loci. In each case, the sgRNAs expose a window where base editing can result in the introduction of point mutations in DNA modifying enzymes that lead to either missense or nonsense mutations. As with the d2GFP editing assay, 24 hrs after transfection, rapamycin (Research Products International) was added to select wells at a final concentration of 200 nM. Transfected cells were harvested at day 3 after transfection, ensuring single-cell suspension. Genomic DNA was collected using the DNeasy Blood & Tissue Kit (Qiagen) according to manufacturer's instructions for sequencing analysis as described below.

### DNA Library Preparation and Sequencing

Target loci of interest were PCR-amplified from 100 ng genomic DNA (primer pairs in Supplementary Sequences) using KAPA HiFi HotStart Uracil+ Ready Mix (Kapa Biosystems) or Phusion High-Fidelity DNA Polymerase (New England Biolabs, NEB). PCR products were then purified (Qiagen).

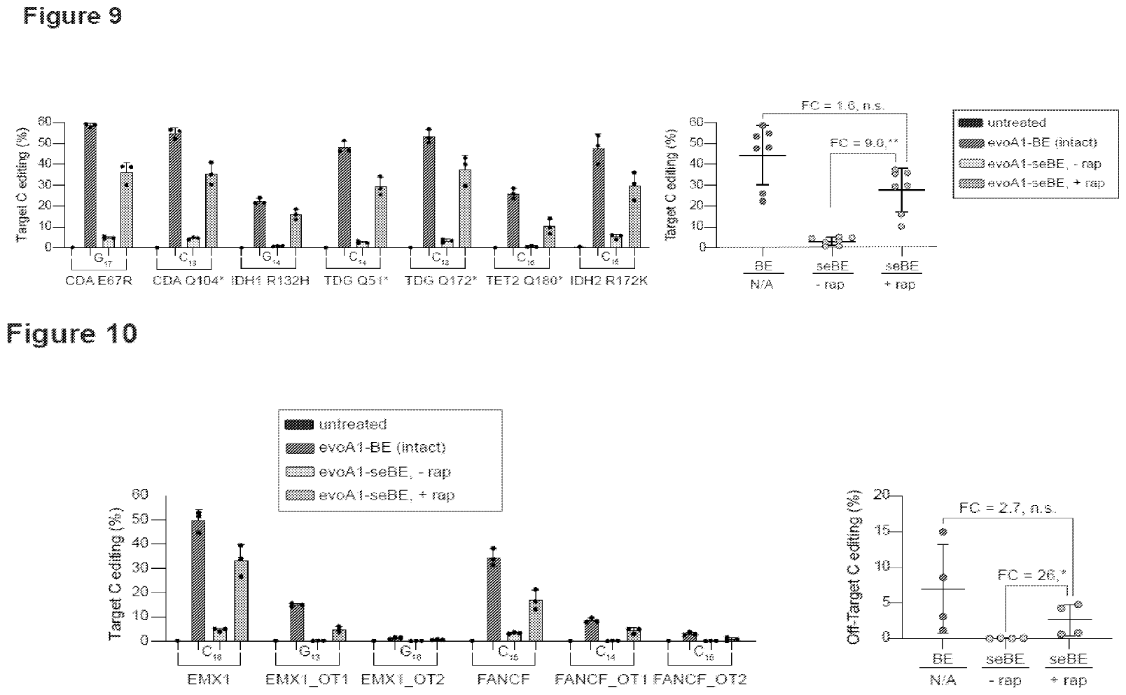

Some samples were deep-sequenced by Amplicon-EZ Next Generation Sequencing (Genewiz). Alternatively, indexed DNA libraries were prepared using the NEBNext Ultra II DNA Library Prep Kit for Illumina with the following specifications. After adapter ligation and 4 cycles of PCR enrichment, indexed amplicon concentration was quantified by Qubit dsDNA HS Assay Kit (ThermoFisher), and size distribution was determined on a Bioanalyzer 2100 (Agilent) with the DNA 1000 Kit (Agilent). Indexed PCR amplicons with different barcodes were pooled together in an equimolar ratio for paired-end sequencing by MiSeq (Illumina) with the 300-cycle MiSeq Reagent Nano Kit v2 (Illumina). Raw reads were automatically demultiplexed by MiSeq Reporter. Demultiplexed read qualities were evaluated by FastQC v0.11.9 as described on the world wide web at bioinformatices.babraham.ac.uk/projects/fastqc. Low-quality sequence (Phred quality score <28) and adapters were trimmed via Trim Galore v0.6.5 as described on the world wide web bioinformatics.babraham.ac.uk/projects/trim_galore/ prior to analysis with CRISPResso2. Sequencing yielded ˜13,000 median aligned reads per sample (5^{th} percentile ˜4,000, 95^{th} percentile ˜63,000). The reported data (FIG. 7 and FIG. 9) represent the frequency of editing at the target base alone, with complete analysis across the sgRNA region.

### RNA Sequencing

Total RNA, isolated as described above, was analyzed for quality using the RNA 6000 Nano Bioanalyzer kit (Agilent). Only RNA with an RNA integrity number (RIN) ≥8 was used for subsequent library construction. RNA-seq was performed on 500 ng-1 μg of total RNA according to the Genewiz Illumina Hi-seq protocol for poly(A)-selected samples (2×150 bp pair-end sequencing, 350M raw reads per lane). The resulting reads were analyzed using the RADAR pipeline (RNA-editing Analysis-pipeline to Decode All twelve-types of RNA-editing events⁵¹. RNA edits that were present in the sgRNA-only samples were removed with analysis performed only on unique editing events present in the samples.

SEQUENCES Suitable for Use in the Base Editing Complexes Described Herein.

All oligonucleotides were purchased from Integrated DNA Technologies (IDT).

Primers used for generating sgRNA transfection plasmids. LRche2.1T vector was used as a template as noted in the methods section.

gRNA_KNB1_top
(SEQ ID NO: 5)
CACCGCAAGCAGAAGAACGGCATCA
gRNA_KNB1_bottom
(SEQ ID NO: 6)
AAACTGATGCCGTTCTTCTGCTTGC

Primers used to add XmaI restriction site to pCMV_ABEmax and pCMV_BE4max.

XmaI ABEmax Forward
(SEQ ID NO: 7)
CTGAGACACCcgggACAAGCGAGAGC
XmaI ABEmax Reverse
(SEQ ID NO: 8)
agccagaggagcctccgc
XmaI BE4max Forward
(SEQ ID NO: 9)
GCGAGACACCcgggACAAGCGAGTC
XmaI BE4max Reverse
(SEQ ID NO: 10)
tgccagaggAtcctccgc

Primers used for generating split BE3, split BE4max and split monomer ABEmax transfection plasmids. The same forward primer (splitCD FRB/FKBP Forward) was used to generate all 5 constructs.

splitCD FRB/FKBP Forward
(SEQ ID NO: 11)
GTCAGATCCGCTAGAGATCCGCGGCCGCTAATACGACTCACTATAGGGAG
AGCCGCCACCATGGAACAAAAACTTATTTCTGAAGAAG
AIDC12 FRB/FKBP BE3 Reverse
(SEQ ID NO: 12)
GTGTGGCGGACTCTGAGGTCCCGGGAGTCTCGCTGCCGCTCAGCAGAATA
CGACGCAGCTG
A3A FRB/FKBP BE3 Reverse
(SEQ ID NO: 13)
TGTGGCGGACTCTGAGGTCCCGGGAGTCTCGCTGCCGCTGTTTCCCTGAT
TCTGGAGAATGG
evorA1 FRB/FKBP BE3 Reverse
(SEQ ID NO: 14)
TGTGGCGGACTCTGAGGTCCCGGGAGTCTCGCTGCCGCTcttcaggcctg
tggccc
AIDC12 FRB/FKBP BE4max Reverse
(SEQ ID NO: 15)
ctggtgttgctgactcgcttgtcccgggtgtctcgctgccagaggatcct
ccgctagatccgccagaCAGCAGAATACGACGCAGCTG
A3A FRB/FKBP BE4max Reverse
(SEQ ID NO: 16)
ctggtgttgctgactcgcttgtcccgggtgtctcgctgccagaggatcct
ccgctagatccgccagaGTTTCCCTGATTCTGGAGAATGG
evorA1 FRB/FKBP BE4max Reverse
(SEQ ID NO: 17)
ctggtgttgctgactcgcttgtcccgggtgtctcgctgccagaggatcct
ccgctagatccgccagacttcaggcctgtggcc
monoABEmax Reverse
(SEQ ID NO: 18)
gtgttgcgctctcgcttgtcccgggtgtctcagagccagaggagcctccg
tcagatcctccggagtcggtggagctctggg

Split Deaminases Gene Block Fragments

Myc-NLS-A3An-FRB-T2A-FKBP12-A3Ac-FlagTag

(SEQ ID NO: 19)
ATGGAACAAAAACTTATTTCTGAAGAAGATCTGAAAAGGCCGGCGGCCAC
GAAAAAGGCCGGCCAGGCAAAAAAGAAAAAGGGAGGTTCCGCTAGCGGAG
GTTCGATGGAAGCCAGCCCAGCATCCGGGCCCAGACACTTGATGGATCCA
CACATATTCACTTCCAACTTTAACAATGGCATTGGAAGGCATAAGACCTA
CCTGTGCTACGAAGTGGAGCGCCTGGACAATGGCACCTCGGTCAAGATGG
ACCAGCACAGGGGCTTTCTACACAACCAGGCTAAGAATCTTCTCTGTGGC
TTTTACGGCCGCCATGCGGAGCTGCGCTTCTTGGACCTGGTTCCTTCTTT
GCAGTTGGACCCGGGCGCGCCgGGAGGTGGTGGCAGCGGTGGAGGAGGTT
CTGGGGGCGGTGGCTCAATTTTATGGCATGAGATGTGGCATGAGGGTTTG
GAAGAGGCATCTAGATTGTATTTCGGTGAAAGAAATGTCAAGGGAATGTT
CGAAGTTTTAGAACCGTTGCACGCTATGATGGAGAGAGGTCCACAGACTC
TAAAGGAGACTTCCTTCAACCAAGCTTATGGAAGGGACCTAATGGAGGCT
CAAGAATGGTGTAGAAAATACATGAAAAGTGGAAATGTAAAGGACCTTAC
ACAAGCTTGGGATCTCTACTACCATGTTTTTAGGAGAATATCTAAAGGAA
GTGGTGAGGGTAGGGGAAGTTTATTAACCTGTGGGGATGTTGAAGAAAAT
CCAGGTCCTATGGGCGTACAAGTTGAAACTATCAGCCCTGGGGACGGCAG
AACCTTTCCGAAGAGGGGACAGACATGTGTTGTTCACTATACTGGAATGT
TGGAAGATGGTAAGAAGTTCGATAGCAGCAGAGATAGGAATAAACCATTT
AAATTCATGCTTGGCAAGCAAGAAGTGATTAGGGGTTGGGAAGAAGGTGT
CGCTCAAATGAGTGTAGGTCAGAGGGCTAAGTTAACAATTAGTCCTGATT
ATGCTTATGGCGCTACAGGTCATCCAGGAATCATTCCCCCACATGCTACT
CTTGTTTTCGACGTTGAATTGCTTAAGCTTGAAGGATCAGGTTCTGGATC
TGGTTCAGGATCAGGCTCACCCGGGCTTGCCCAGATCTACAGGGTCACCT
GGTTCATCTCCTGGAGCCCCTGCTTCTCCTGGGGCTGTGCCGGGGAAGTG
CGTGCGTTCCTGCAGGAGAACACACACGTGAGACTGCGTATCTTCGCTGC
CCGCATCTATGATTACGACCCCCTATATAAGGAGGCACTGCAAATGCTGC
GGGATGCTGGGGCCCAAGTCTCCATCATGACCTACGATGAATTTAAGCAC
TGCTGGGACACCTTTGTGGACCACCAGGGATGTCCCTTCCAGCCCTGGGA
TGGACTAGATGAGCACAGCCAAGCCCTGAGTGGGAGGCTGCGGGCCATTC
TCCAGAATCAGGGAAACGGTACCGGGTCGGGTAGTGGCTCTGGTAGTGGT
TCTGGTTCTGATTACAAAGACGATGACGATAAGTAA

Myc-NLS-AIDn-FRB-T2A-FKBP12-AID-FlagTag

(SEQ ID NO: 20)
ATGGAACAAAAACTTATTTCTGAAGAAGATCTGAAAAGGCCGGCGGCCAC
AGAAAAGGCCGGCCAGGCAAAAAAGAAAAAGGGAGGTTCCGCTAGCGGAG
TGTCGATGGATAGCCTGCTGATGAACCGTCGTgAATTTCTGTATCAGTTT
AAAAACGTGCGTTGGGCGAAAGGCCGTCGTGAAACCTATCTGTGCTATGT
GGTGAAACGTCGTGATAGCGCGACCAGCTTTAGCCTGGATTTTGGCTATC
TGCGTAACAAAAACGGCTGCCATGTGGAACTGCTGTTTCTGCGTTATATT
AGCGATTGGGATCTGGATCCGGGCGCGCCgGGAGGTGGTGGCAGCGGTGG
AGGAGGTTCTGGGGGCGGTGGCTCAATTTTATGGCATGAGATGTGGCATG
AGGGTTTGGAAGAGGCATCTAGATTGTATTTCGGTGAAAGAAATGTCAAG
GGAATGTTCGAAGTTTTAGAACCGTTGCACGCTATGATGGAGAGAGGTCC
ACAGACTCTAAAGGAGACTTCCTTCAACCAAGCTTATGGAAGGGACCTAA
TGGAGGCTCAAGAATGGTGTAGAAAATACATGAAAAGTGGAAATGTAAAG
GACCTTACACAAGCTTGGGATCTCTACTACCATGTTTTTAGGAGAATATC
TAAAGGAAGTGGTGAGGGTAGGGGAAGTTTATTAACCTGTGGGGATGTTG
AAGAAAATCCAGGTCCTATGGGCGTACAAGTTGAAACTATCAGCCCTGGG
GACGGCAGAACCTTTCCGAAGAGGGGACAGACATGTGTTGTTCACTATAC
TGGAATGTTGGAAGATGGTAAGAAGTTCGATAGCAGCAGAGATAGGAATA
AACCATTTAAATTCATGCTTGGCAAGCAAGAAGTGATTAGGGGTTGGGAA
GAAGGTGTCGCTCAAATGAGTGTAGGTCAGAGGGCTAAGTTAACAATTAG
TCCTGATTATGCTTATGGCGCTACAGGTCATCCAGGAATCATTCCCCCAC
ATGCTACTCTTGTTTTCGACGTTGAATTGCTTAAGCTTGAAGGATCAGGT
TCTGGATCTGGTTCAGGATCAGGCTCACCCGGGCTTGGCCGTTGCTATCG
TGTGACCTGGTTTAtCAGCTGGAGCCCGTGCTATGATTGCGCGCGTCATG
TGGCGGATTTTCTGCGTGGCAACCCGAACCTGAGCCTGCGTATTTTTACC
GCGCGTCTGTATTTTTGCGAAgCcGgcaGgCGtGAACCGGAAGGCCTGCG
TCGTCTGCATCGTGCGGGCGTGCAGATTGCGATTATGACCTTTAAAGATT
ATTTTTATTGCTGGAACACCTTTGTGGAAAACCATGgACGTACCTTTAAA
GCGTGGGAAGGCCTGCATGAAAACAGCGTGCGTCTGAGCCGTCAGCTGCG
ATCGTATTCTGCTGGGTACCGGGTCGGGTGTGGCTCTGGTAGTGGTTCTG
GTTCTGATTACAAAGACGATGACGATAAGTAA

Myc-NLS-evorA1n-FRB-T2A-FKBP12-evorA1c

(SEQ ID NO: 21)
ATGGAACAAAAACTTATTTCTGAAGAAGATCTGAAAAGGCCGGCGGCCAC
GAAAAAGGCCGGCCAGGCAAAAAAGAAAAAGGGAGGTTCCGCTAGCGGAG
GTTCGATGAGTTCAAAGACTGGGCCTGTCGCCGTCGATCCAACCCTGCGC
CGCCGGATTGAACCTCACGAGTTTGAAGTGTTCTTTGACCCCCGGGAGCT
GAGAAAGGAGACATGCCTGCTGTACGAGATCAACTGGGGAGGCAGGCACT
CCATCTGGAGGCACACCTCTCAGAACACAAATAAGCACGTGGAGGTGAAC
TTCATCGAGAAGTTTACCACAGAGCGGTACTTCTGCCCCGGCGCGCCGGG
AGGTGGTGGCAGCGGTGGAGGAGGTTCTGGGGGCGGTGGCTCAATTTTAT
GGCATGAGATGTGGCATGAGGGTTTGGAAGAGGCATCTAGATTGTATTTC
GGTGAAAGAAATGTCAAGGGAATGTTCGAAGTTTTAGAACCGTTGCACGC
TATGATGGAGAGAGGTCCACAGACTCTAAAGGAGACTTCCTTCAACCAAG
CTTATGGAAGGGACCTAATGGAGGCTCAAGAATGGTGTAGAAAATACATG
AAAAGTGGAAATGTAAAGGACCTTACACAAGCTTGGGATCTCTACTACCA
TGTTTTTAGGAGAATATCTAAAGGAAGTGGTGAGGGTAGGGGAAGTTTAT
TAACCTGTGGGGATGTTGAAGAAAATCCAGGTCCTATGGGCGTACAAGTT
GAAACTATCAGCCCTGGGGACGGCAGAACCTTTCCGAAGAGGGGACAGAC
ATGTGTTGTTCACTATACTGGAATGTTGGAAGATGGTAAGAAGTTCGATA
GCAGCAGAGATAGGAATAAACCATTTAAATTCATGCTTGGCAAGCAAGAA
GTGATTAGGGGTTGGGAAGAAGGTGTCGCTCAAATGAGTGTAGGTCAGAG
GGCTAAGTTAACAATTAGTCCTGATTATGCTTATGGCGCTACAGGTCATC
CAGGAATCATTCCCCCACATGCTACTCTTGTTTTCGACGTTGAATTGCTT
AAGCTTGAAGGATCAGGTTCTGGATCTGGTTCAGGATCAGGCTCACCCGG
GCTTAATACCAGATGTAGCATCACATGGTTTCTGAGCTGGTCCCCTTGCG
GAGAGTGTAGCAGGGCCATCACCGAGTTCCTGTCCAGATATCCAAATGTG
ACACTGTTTATCTACATCGCCAGGCTGTATCACCTGGCAAACCCAAGGAA
TAGGCAGGGCCTGCGCGATCTGATCAGCTCCGGCGTGACCATCCAGATCA
TGACAGAGCAGGAGTCCGGCTACTGCTGGCACAACTTCGTGAATTATTCT
CCTAGCAACGAGTCCCACTGGCCTAGGTACCCACACCTGTGGGTGCGCCT
GTACGTGCTGGAGCTGTATTGCATCATCCTGGGCCTGCCCCCTTGTCTGA
ATATCCTGCGGAGAAAGCAGAGCCAGCTGACCTCCTTTACAATCGCCCTG
CAGTCTTGTCACTATCAGAGGCTGCCACCCCACATCCTGTGGGCCACAGG
CCTGAAG

Myc-NLS-TadAn-FRB-T2A-FKBP12-TadAc

(SEQ ID NO: 22)
ATGGAACAAAAACTTATTTCTGAAGAAGATCTGAAAAGGCCGGCGGCCAC
GAAAAAGGCCGGCCAGGCAAAAAAGAAAAAGGGAGGTTCCGCTAGCGGAG
GTTCGATGtctgaggtggagttttcccacgagtactggatgagacatgcc
ctgaccctggccaagagggcacgcgatgagagggaggtgcctgtgggagc
cgtgctggtgctgaacaatagagtgatcggcgagggctggaacagagcca
tcggcctgcacgacccaacagcccatgccgaaattatggccctgagacag
ggcggcctggtcatgcagaactacagactgGGCGCGCCgGGAGGTGGTGG
CAGCGGTGGAGGAGGTTCTGGGGGCGGTGGCTCAATTTTATGGCATGAGA
TGTGGCATGAGGGTTTGGAAGAGGCATCTAGATTGTATTTCGGTGAAAGA
AATGTCAAGGGAATGTTCGAAGTTTTAGAACCGTTGCACGCTATGATGGA
GAGAGGTCCACAGACTCTAAAGGAGACTTCCTTCAACCAAGCTTATGGAA
GGGACCTAATGGAGGCTCAAGAATGGTGTAGAAAATACATGAAAAGTGGA
AATGTAAAGGACCTTACACAAGCTTGGGATCTCTACTACCATGTTTTTAG
GAGAATATCTAAAGGAAGTGGTGAGGGTAGGGGAAGTTTATTAACCTGTG
GGGATGTTGAAGAAAATCCAGGTCCTATGGGCGTACAAGTTGAAACTATC
AGCCCTGGGGACGGCAGAACCTTTCCGAAGAGGGGACAGACATGTGTTGT
TCACTATACTGGAATGTTGGAAGATGGTAAGAAGTTCGATAGCAGCAGAG
ATAGGAATAAACCATTTAAATTCATGCTTGGCAAGCAAGAAGTGATTAGG
GGTTGGGAAGAAGGTGTCGCTCAAATGAGTGTAGGTCAGAGGGCTAAGTT
AACAATTAGTCCTGATTATGCTTATGGCGCTACAGGTCATCCAGGAATCA
TTCCCCCACATGCTACTCTTGTTTTCGACGTTGAATTGCTTAAGCTTGAA
GGATCAGGTTCTGGATCTGGTTCAGGATCAGGCTCACCCGGGCTTattga
cgccaccctgtacgtgacattcgagccttgcgtgatgtgcgccggcgcca
tgatccactctaggatcggccgcgtggtgtttggcgtgaggaacgcaaaa
accggcgccgcaggctccctgatggacgtgctgcactaccccggcatgaa
tcaccgcgtcgaaattaccgagggaatcctggcagatgaatgtgccgccc
tgctgtgctatttctttcggatgcctagacaggtgttcaatgctcagaag
aaggcccagagctccaccgacGGTACCGGGTCGGGTAGTGGCTCTGGTAG
TGGTTCTGGTTCTGATTACAAAGACGATGACGATAAGTAA

Primers Used for d2GFP Loci Sequencing:

d2GFP forward primer 1:
(SEQ ID NO: 23)
CTTCAAGGAGGACGGCAAC
d2GFP reverse primer 1:
(SEQ ID NO: 24)
GTGGTCGGCGAGCTG

d2GFP Sequence

(SEQ ID NO: 25)
ATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAGG
GCGAGGGCGATGCCACCTACGGCAAGCTGACCCTGAAGTTCATCTGCACC
ACCGGCAAGCTGCCCGTGCCCTGGCCCACCCTCGTGACCACCCTGACCTA
CGGCGTGCAGTGCTTCAGCCGCTACCCCGACCACATGAAGCAGCACGACT
TCTTCAAGTCCGCCATGCCCGAAGGCTACGTCCAGGAGCGCACCATCTTC
TTCAAGGACGACGGCAACTACAAGACCCGCGCCGAGGTGAAGTTCGAGGG
CGACACCCTGGTGAACCGCATCGAGCTGAAGGGCATCGACTTCAAGGAGG
ACGGCAACATCCTGGGGCACAAGCTGGAGTACAACTACAACAGCCACAAC
GTCTATATCATGGCCGACAAGCAGAAGAACGGCATCAAGGTGAACTTCAA
GATCCGCCACAACATCGAGGACGGCAGCGTGCAGCTCGCCGACCACTACC
AGCAGAACACCCCCATCGGCGACGGCCCCGTGCTGCTGCCCGACAACCAC
TACCTGAGCACCCAGTCCGCCCTGAGCAAAGACCCCAACGAGAAGCGCGA
TCACATGGTCCTGCTGGAGTTCGTGACCGCCGCCGGGATCACTCTCGGCA
TGGACGAGCTGTACAAGAAGCTTAGCCATGGCTTCCCGCCGGAGGTGGAG
GAGCAGGATGATGGCACGCTGCCCATGTCTTGTGCCCAGGAGAGCGGGAT
GGACCGTCACCCTGCAGCCTGTGCTTCTGCTAGGATCAATGTGTAG

Linker-GFP-Linker Sequence

(SEQ ID NO: 26)
GGATCCGCTGGCTCCGCTGCTGGTTCTGGCGAATTCATGAGCAAAGGAGA
AGAACTTTTCACTGGAGTTGTCCCAATTCTTGTTGAATTAGATGGTGATG
TTAATGGGCACAAATTTTCTGTCAGAGGAGAGGGTGAAGGTGATGCTACA
ATCGGAAAACTCACCCTTAAATTTATTTGCACTACTGGAAAACTACCTGT
TCCATGGCCAACACTTGTCACTACTCTGACCTATGGTGTTCAATGCTTTT
CCCGTTATCCGGATCACATGAAAAGGCATGACTTTTTCAAGAGTGCCATG
CCCGAAGGTTATGTACAGGAACGCACTATATCTTTCAAAGATGACGGGAA
ATACAAGACGCGTGCTGTAGTCAAGTTTGAAGGTGATACCCTTGTTAATC
GTATCGAGTTAAAGGGTACTGATTTTAAAGAAGATGGAAACATTCTCGGA
CACAAACTCGAGTACAACTTTAACTCACACAATGTATACATCACGGCAGA
CAAACAAAAGAATGGAATCAAAGCTAACTTCACAGTTCGCCACAACGTTG
AAGATGGTTCCGTTCAACTAGCAGACCATTATCAACAAAATACTCCAATT
GGCGATGGCCCTGTCCTTTTACCAGACAACCATTACCTGTCAACACAAAC
TGTCCTTTCGAAAGATCCCAACGAAAAGGGTACCCGTGACCACATGGTCC
TTCATGAGTCTGTAAATGCTGCTGGGATTACAGGTGGAGGAGGTTCTGGA
GGCGGTGGAAGTGGTGGCGGAGGTAGC

Primers used to clone AIDn fragments. AIDn Forward primer was used to generate all AIDn fragments. Select sequence for insert 2 are shown as these were the sites carried forward.

AIDn Forward
(SEQ ID NO: 27)
tcgaaaacctgtattttcaggggtcgacaATGGATAGCCTGCTG
AIDn Reverse - (insert 2)
(SEQ ID NO: 28)
CAGCGGATCCCGGATCCAGATCCCAATCGC

Primers used to clone AIDc fragments. AIDc Reverse primer was used to generate all AIDc fragments.

AIDc Forward - (insert 2)
(SEQ ID NO: 29)
CGGAGGTAGCGGCCGTTGCTATCGTGTG
AIDc Reverse
(SEQ ID NO: 30)
gggctttgtttagcagcctaggCTACAGGCCCAGGGTAC

Primers used to clone linker-GFP-linker fragments.

Linker-GFP-Linker B1/B2 Forward
(SEQ ID NO: 31)
TCTGGATCCGGGATCCGCTGGCTCCGC
Linker-GFP-Linker B1/B2 Reverse
(SEQ ID NO: 32)
AGCAACGGCCGCTACCTCCGCCACCACTTC

Primers Used for Overlap Extension PCR

AIDn Forward
(SEQ ID NO: 33)
Cagaattcgaaaacctgtattttcag
AIDc Reverse
(SEQ ID NO: 34)
Ctttcgggctttgtttagcagcc

The following examples are provided to illustrate certain embodiments of the invention. They are not intended to limit the invention in any way.

### EXAMPLE I

### Generation of a Split DNA Deaminase and Use Thereof for Controlled Base Editing

DNA deaminase enzymes have been converted into efficient and controllable genome editors, thereby overcoming constraints that will otherwise limit their scientific and therapeutic potential.

Members of the zinc-dependent nucleic acid deaminase family have evolved distinctively to act on a variety of substrates serving different biological roles, while retaining the same core structure. Activation induced deaminase (AID) mutates cytosine bases to uracil in the immunoglobulin locus of B-cells, initiating somatic hypermutation and antibody maturation. Related APOBEC3 DNA deaminases mutate and restrict foreign retroviruses, and more distantly related deaminases can even act on adenosine in tRNA. Nature's enzymatic toolbox for introducing base transition mutations, while powerful, has been subjected to several evolutionary requirements, given the threat that purposeful mutators pose to genomic stability. These requirements include constrained sub-optimal deaminase activity and several layers of regulatory control. Despite these constraints, DNA deaminases can act aberrantly on the genome when mis-regulated, and their activity is known to contribute to genomic instability and to promote cancer mutagenesis.

The ability to target DNA deaminases to specific loci has opened up new frontiers with the potential to transform biology and medicine by allowing for precise gene editing without introducing double-stranded DNA (dsDNA) breaks. In the base editing complex, catalytically-inactive Cas9 (dCas9) is partnered with a DNA deaminase. Unable to generate dsDNA breaks, dCas9 functions as a ‘genomic GPS’ bringing the deaminase to a specific locus dictated by a single-guide RNA (sgRNA), where dCas9 binding also exposes a window of single-stranded DNA (ssDNA) that can then be edited by the DNA deaminase. The tethered DNA deaminase can then act on the exposed single-stranded DNA to induce C:G to T:A mutations in the case of AID/APOBEC cytosine base editors (CBEs) or A:T to G:C mutations with evolved TadA adenosine base editors (ABEs)^{4, 5}. In the case of CBEs, the fusion of one or more protein inhibitors of uracil repair (UGIs) further promotes C:G to T:A transitions over other outcomes⁶. Alternatively, more processive DNA deaminases can facilitate targeted diversification in place of precise transition mutations^{7, 8}. In their physiological roles in immune defense, AID/APOBEC enzymes are highly regulated at multiple levels, including via transcriptional control, alternative splicing, post-translational modification, and interaction partners^{9, 10}. Efficient regulation is imperative, as DNA deaminases also pose risks to the genome^{11, 12}. Mistargeting of AID and its APOBEC3 (A3) relatives results in mutations and translocations in a variety of cancers¹³⁻¹⁷. These known pathological activities help explain why BEs, which contain unregulated deaminases, have more recently been shown to have significant sgRNA-independent off-target activities. Indeed, genome-wide transition mutations occur more frequently after CBE or ABE exposure, and transcriptome-wide mutations increase due to off-target deaminase activity on RNA¹⁸⁻²³.

Although different AID/APOBEC family members have been explored, initial efforts largely focused on rat APOBEC1 as the base editor, in concert with accessory modules that skew downstream repair pathways to favor the desired transition mutations. Notably, while mutations can be localized within the ssDNA exposed by dCas9, editing efficiency remains a major challenge.

Current strategies have increased efficiency by using a nickase-Cas9 (nCas9), but at the cost of imprecision, tolerating more insertions/deletions (indels). Furthermore, recent work has uncovered substantial off-target effects from deaminases, which can mutate DNA or RNA independent of Cas9 binding. Both the power and challenges of base editing are captured by recent advances in the correction of pathologic point mutations, the generation of knockouts via targeted stop codon introduction, and broad applications in discovery platforms in the lab. In such settings, base editing can be used to great effect, but can also lead to off-target action, given the absence of regulatory control over the editing enzymes.

Inducible editing activity of split engineered base editors is described in the present example. Our strategy for moving to controllable mammalian base editing complexes involves use of molecules which are capable of dimerization in response to dimerization inducing molecules, for example the rapamycin-regulated dimerization of FKBP and FRB. In this system, proteins linked to FKBP and FRB (e.g., portions of a split deaminase) can be approximated to one another by the addition of rapamycin or related analogs (rapalogs). The seBEs described herein link the split deaminase elements with the targeting dCas9 module, although many possible permutations are described and are shown in the Figures.

To advance towards a split DNA deaminase, we looked to precedents from the larger deaminase family that share a characteristic α/β deaminase fold²⁷, including pyrimidine salvage enzymes that have been split via rational manipulation of loop regions²⁸. Our strategy involved two steps: identifying sites that tolerate insertion of GFP, and then splitting GFP to test if the DNA deaminase can be split and spontaneously reconstituted. Building on the known structure of AID²⁹, we focused first on a variant containing several hyperactivating mutations^{30, 31} (AID*) that could potentiate efficient genome editing. We targeted five loops in AID* for insertion (FIG. 2A). Three constructs (AID*-INS1-3) target core enzyme loops, each with an insertion of an evolved GFP variant (optGFP³²). Additionally, we inserted optGFP into the active site loop (β3-α3) as a negative control (AID*-INS−) that abolishes deaminase activity and into the dispensable³³ C-terminal loop as a positive control (AID*-INS+).

To test for insertional tolerance, we expressed constructs in E. coli and measured deaminase activity with a rifampin-based mutagenesis assay. In this assay, DNA deaminase expression promotes untargeted mutagenesis, and the frequency of acquired rifampin resistance (Rif^{R}) is a well-established means to assess overall deaminase activity^{30, 34}. Using this approach, AID(WT) expression increases Rif^{R} 12-fold relative to a catalytically inactive mutant AID(E58A), while hyperactive AID* shows a 265-fold Rif^{R} increase (FIG. 2B). As predicted, AID*-INS− shows compromised mutator activity, while AID*-INS+ produces AID*-like activity. Turning to the core insertion variants, either β1-β2 (AID*-INS1) or α3-β4 (AID*-INS3) insertion was tolerated, but with significantly reduced activity. Promisingly, however, AID*-INS2 (α2-β3) showed activity comparable to AID* alone, suggesting that the enzyme scaffold is tolerant to the introduction of a protein domain at this location.

We hypothesized that strategies may differ based on the location of the tolerated split in the DNA deaminase, which will in turn influence choice of linkers and the order of linkage between the different elements in the editing complex. Having demonstrated insertion tolerance, we next evaluated if the insertion tolerant site could be used to split the DNA deaminase. We had initially inserted optGFP because this variant can be used to split GFP in the loop between the last two β-strands (β₁₀-β₁₁), with co-expression of two fragments leading to spontaneous GFP reconstitution³². With therefore next split AID*-INS2 between β₁₀ and β₁₁ of optGFP, resulting in a construct pair of AID*_{N}-optGFP₁₋₁₀ (AID*-SPL2_{N}) and GFP₁₁-AID*_{C} (AID*-SPL2_{C}). As predicted, either AID* fragment alone showed no increase in Rif^{R} (FIG. 2B). As the kinetics of optGFP reassembly are not conducive to the Rif^{R} E. coli assay, we next co-expressed the AID*-SPL2_{N} and AID*-SPL2_{C} to address if the fragments could spontaneously reconstitute into active enzyme (FIG. 2E). We purified the reconstituted protein complex (AID*-SPL2) from E. coli and observed visible fluorescence, suggesting spontaneous GFP assembly (FIG. 2F). To test for enzymatic activity, we used an in vitro assay that can report on a single C→U change, based on fragmentation of a single-stranded DNA oligonucleotide (FIG. 2G). We found that AID*-SPL2 showed deaminase activity comparable to that of AID*-INS2 and only ˜4-fold reduced from that of intact AID*. These results support the AID*loop as a split site for generating inactive deaminase fragments that can be reconstituted.

Given the shared structural architecture of AID/APOBEC family enzymes, we hypothesized that the α2-β3 loop might prove to be a generalizable split site. To this end, we examined if human A3 enzyme APOBEC3A (A3A)^{25, 35, 36} could also be split into two inactive fragments that can be reconstituted. We first validated that A3A tolerated optGFP insertion at its α2-β3 loop in vitro (FIG. 3A and FIG. 3B) and then turned to examine activity in mammalian cells. A3A expression can induce the DNA damage response (DDR), as detected by phosphorylation of histone variant H2AX (γH2AX)³⁷. Accordingly, we analyzed the DDR in HEK293T cells transfected with mammalian expression vectors containing A3A-INS2, catalytically inactive mutant A3A(E72A)-INS2, or the two split fragments A3A-SPL2_{N} and A3A-SPL2_{C} (FIG. 3C). Post-transfection, GFP⁺ cells expressing A3A-INS2 showed increased γH2AX relative to A3A(E72A)-INS2. For cells co-expressing A3A-SPL2_{N} and A3A-SPL2_{C}, we observed GFP reassembly and readily detected γH2AX by both flow cytometry and immunofluorescence microscopy (FIG. 3D and FIG. 3E). These results support α2-β3 as a viable split site across the DNA deaminase family and highlight the feasibility of manipulating this site to achieve regulatory control over deaminase activity.

Our controllable split-engineered base editor (seBE) design requires a transition from spontaneous split GFP reassembly to switchable chemical-induced protein dimerization (CID) of deaminase fragments. To achieve CID, we employed the common rapamycin-regulated heterodimerization of FK506 binding protein 12 (FKBP12) and FKBP rapamycin binding domain (FRB)³⁸. To explore generalizability of the seBE strategy, we generated three distinct seBE variants in the scaffold of BE4max³⁹, containing an alternative hyperactive variant of AID (AID′), evolved APOBEC1 (evoA1), or A3A followed by Cas9 nickase (nCas9) and tandem UGIs. The distinctive features of these deaminase variants can permit exploration of different applications: AID is processive and primed for diversity generation⁷, evoA1 has been shown to be highly precise⁴⁰, and A3A demonstrates high C to T conversion efficiency^{25, 35, 36}. Starting from intact BE4max scaffolds, we created seBEs by inserting an artificial gene encoding FRB and FKBP12 at the loop between α2 and β3 with fragments separated by a T2A self-cleaving polypeptide (FIG. 5 and FIG. 6). The resulting constructs thus co-express two fragments: one containing the DNA deaminase N-terminus and FRB; the second containing FKBP12, the DNA deaminase C-terminus, nCas9, and two UGIs in series.

To measure editing efficiency, we derived a HEK293T reporter cell line with a single copy of destabilized GFP (d2GFP) stably integrated (FIG. 4). When d2gfp is targeted, successful base editing can generate a nonsense mutation at Q158 measurable by flow cytometry (GFP^{off}) (FIG. 7). For the intact AID′-BE4max, minimal GFP^{off} cells were observed in the absence of a targeting sgRNA, but editing was highly efficient in its presence (47±6%). With AID′-seBE4max, targeting sgRNA, and no rapamycin, we observed near background levels of GFP^{off} (7±2%). Upon rapamycin addition, we observed robust GFP inactivation (35±10%) indicative of successful CID. The observed patterns were mirrored with evoA1 and A3A constructs, with rapamycin-dependent detection of GFP^{off} cells to levels less approaching that of the intact BE4max counterparts (FIG. 7C).

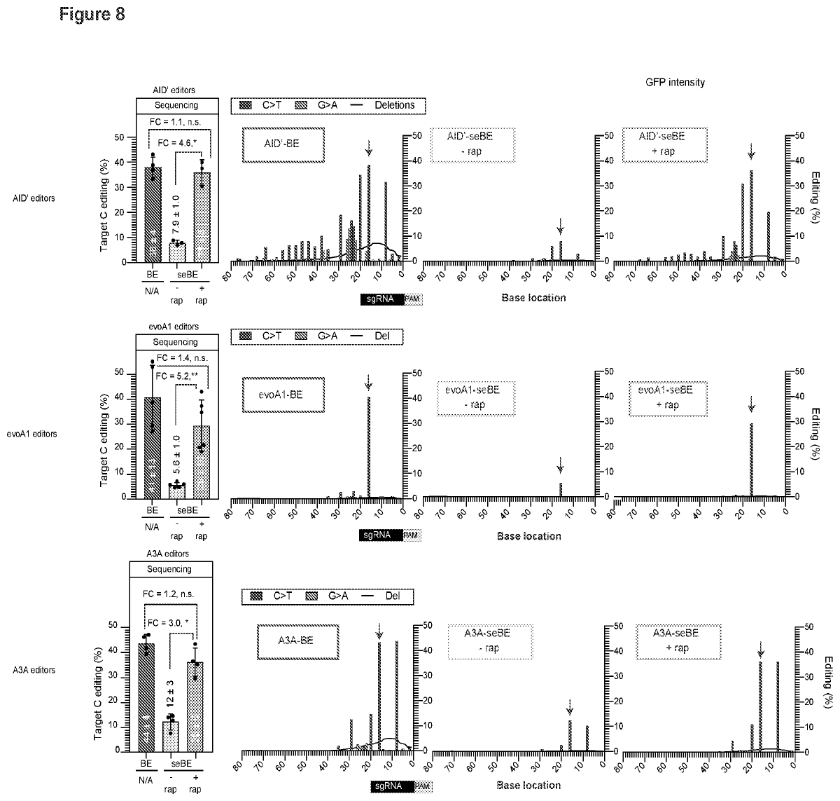

To more rigorously assess editing footprints, we deep-sequenced the d2gfp locus for each condition (FIG. 8). For intact AID′-BE4max, the target cytosine within the Q158 codon showed the highest editing percentage within the locus (38±4%). However, clones also harbored multiple bystander mutations, including indels (7.6±1.4%) and G→A mutations, suggesting editor activity on the sgRNA target strand and showcasing the known processive behavior of AID^{7, 41}. For AID′-seBE4max, we observed low levels of editing at the target base in the absence of rapamycin (8±1%) with marked elevation in its presence (36±5%). The mutational footprint of the seBE appeared similar to the intact editor, albeit with fewer cumulative indels (2.2±0.3%). We also observed controllable editing with the evoA1 series, with the distinction that these editors are more precise rather than processive. With evoA1-seBEmax, rapamycin addition induced editing 5.6-fold, reaching a maximal level 1.4-fold reduced from that of the intact evoA1-BE4max (FIG. 6). Rapamycin-dependent editing extended to the A3A-based editors as well (FIG. 8), demonstrating that small-molecule-regulated base editing is generalizable across multiple seBE constructs.

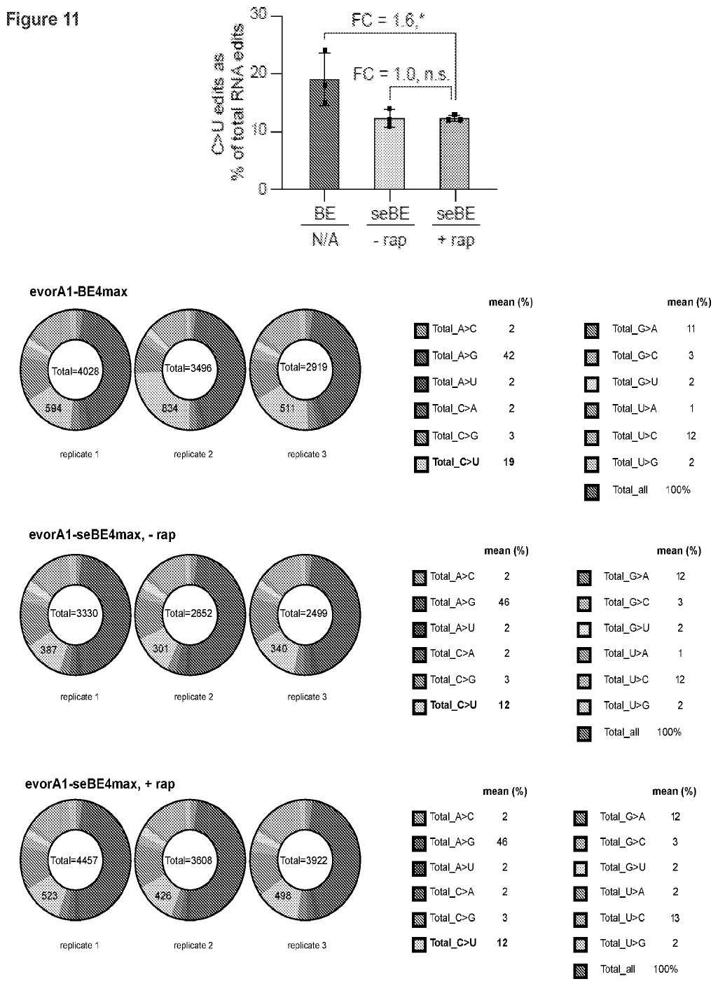

We next aimed to explore whether seBEs permit controllable editing for alternative targets across the genome. We focused our analysis on APOBEC1 constructs given their observed precision and frequent application in the field. We first targeted seven loci involving epigenetic regulators and analyzed on-target base editing efficiency with seBE4max and BE4max constructs. Across sites, the intact evoA1-BE4max average editing efficiency was 44% (FIG. 9). For evoA1-seBE4max in the absence of rapamycin, editing across sites was detectable but low (mean 3%). Upon CID with rapamycin, base editing activity was induced across constructs (mean 28%). On average, base editing was induced 8.2-fold by rapamycin and reached 64% of the editing efficiency achieved by unregulated intact editors. We also extended analysis to two sites, EMX1 and FANCF, with sgRNAs that have well-established genomic off-target sites^{18, 42} (FIG. 10). Editing at sgRNA-dependent off-target sites was nearly absent without rapamycin, but reached 37% of the level of intact evoA1-BE4max upon addition of rapamycin. To probe sgRNA-independent off-target activity, we also performed RNA-seq on samples undergoing d2gfp editing without enrichment or sorting. While transcriptome-wide mutations with intact evoA1-BE4max were lower than those previously reported with BE3-based editors²², we noted elevated frequency and fraction of C→U mutations (FIG. 11). With evoA1-seBE4max, we observed no significant change in C→U mutations, either in the presence or absence of rapamycin, supporting the possibility that evoA1-seBE4max can reduce sgRNA-independent activities associated with expression of an unregulated deaminase.

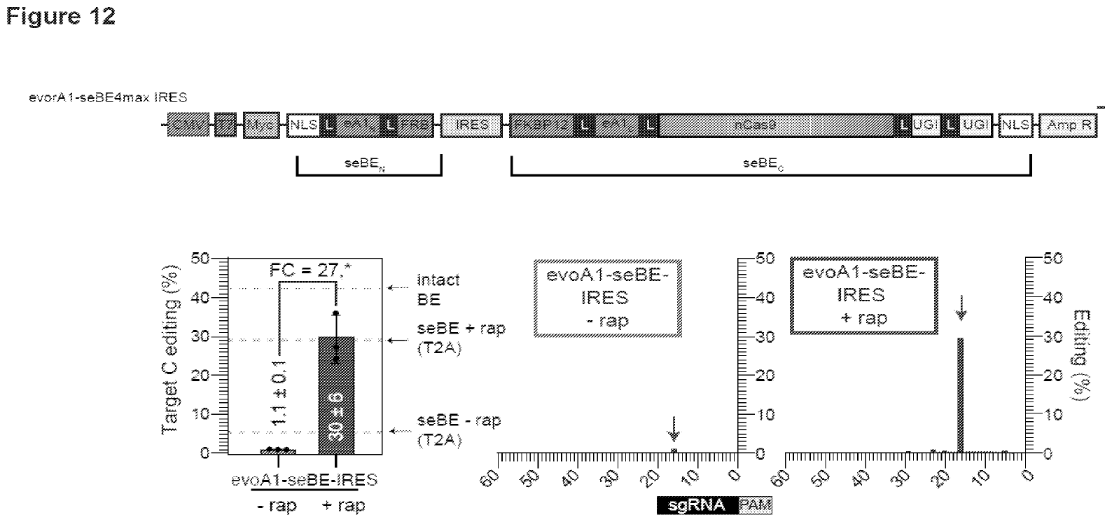

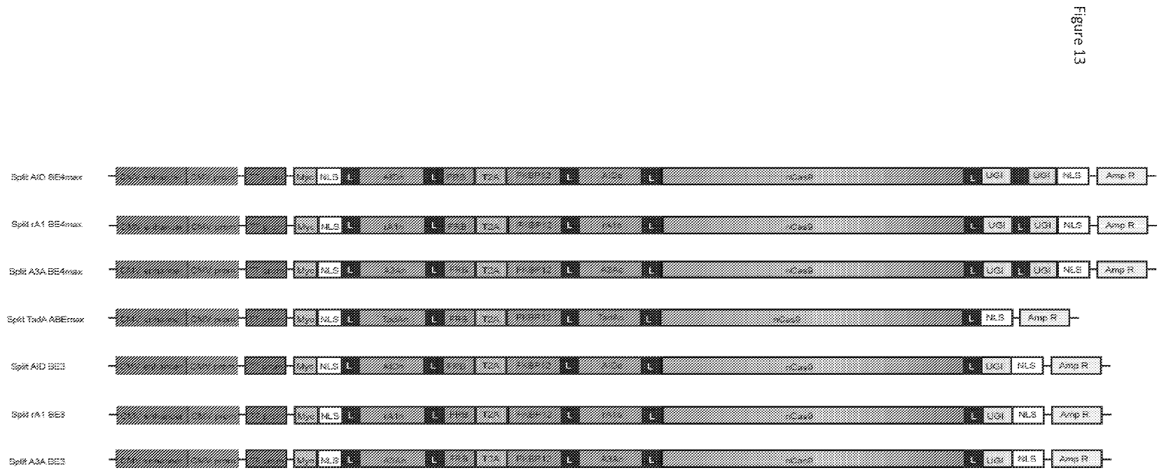

A strength of the seBE strategy is that the system is well poised for modifications to alter either the nature or the degree of regulatory control. For example, we noted that while editing was readily induced by rapamycin with the seBEs, low-level activity was still observable in the absence of rapamycin. We hypothesized that this editing could have resulted from incomplete ribosome skipping with the T2A self-cleaving peptide, which would yield an intact editor. To further increase the dynamic range of small-molecule inducible editing, we generated an evoA1-seBE4max-IRES construct, where the two polypeptides were expressed from two independent promoters, one from a CMV promoter and the other from an internal ribosome entry sequence (IRES) (FIG. 12). Indeed, sequencing analysis revealed that the IRES seBE construct greatly reduced editing in the absence of rapamycin (1.1±0.1%) compared to the T2A construct (5.5±2.3%). Meanwhile, rapamycin-dependent editing remained robust (30±6%) (FIG. 12). Thus, increasing the stringency with which split fragments are expressed separately can readily permit >26-fold inducible control over base editing. Further improvements to alter split fragment complementation, relative levels, or localization can likely allow control to be further tuned or optimized for given applications.

Notably, split deaminases can address multiple off target problems: (1) the existence of an unregulated, constitutively active deaminase that can mutate sites beyond the one targeted by dCas9 and (2) binding of dCas9 to sites outside of the intended sgRNA target. Our seBE-a strategy allows for temporal deaminase control. In cases where off target activity is to be minimized in seBE-a constructs, nuclear localization signals (NLS) can be introduced into either or both constructs perturbing localization and thereby reducing off-target RNA deamination activity. Next, in our seBEb design, we will exploit split Cas9, whereby a Cas9N-FKBP and FRB-Cas9C can be successfully approximated with rapamycin (71). The seBE-b constructs (FIG. 5, FIG. 6, FIG. 13) will for example employ seBE-b1: AIDC-dCas9N-FKBP and seBEb2:FRB-dCas9C-AIDN, which simultaneously maintains the overall architecture of both split Cas9 and successful base editors. Notably, seBE-a strategies are preferred as our earlier studies suggest dimerization may be reversible, while prior work with split Cas9 suggests it may not. The final strategy, seBE-c (FIG. 14), will utilize co-localization with two distinct dCas9/sgRNAs, for enhanced specificity. seBE-c1 will be identical to seBE-a1, while its partner will be seBE-c2: e.g. AIDN-FRB-dCas9. In this construct, the orientation and linkers will be employed which promote preferred action of reconstituted deaminase on the editing window exposed by seBE-c1.

While we have already shown success in the generation of split enzyme base editors (see FIG. 7), a key aspect of this invention is the generalizability of this approach. Given that feasible split sites have been found, not limited to but consistently demonstrated with the loop between α2-β3, the various base editor scaffolds are all amenable to the same strategy.

The generalizability of this strategy is captured in FIG. 13, showing an array of constructs that have been made using this general strategy. Although many more permutations of base editor constructs are possible (see FIG. 5), in this case we are shown different base editor scaffold including that of the adenine base editors (ABEs) which can be used for A to G changes.

In sum, we have demonstrated a generalizable strategy for small-molecule regulation over DNA deaminase activity. Although we focus on BE applications, these split sites could be used to study conditional control over isolated DNA deaminases, as in antibody somatic hypermutation or cancer mutagenesis. Given that the α2-β3 loop tolerates insertion of either split GFP or FKBP/FRB, we anticipate extensions to other CID strategies, such as those using rapalogs, abscisic acid, or photo-inducible protein dimerization systems²⁴. seBEs are also anticipated to function with editor scaffolds beyond BE4max, including those using Cas proteins other than nCas9, or with two different targeting modules to minimize sgRNA-dependent off-target activities, akin to recently developed split dsDNA deaminase editors⁴³ or the dimeric Cas9-FokI heterodimerization systems⁴⁴. Finally, we note that small-molecule inducible seBEs could allow for the potentially powerful ability to controllably induce base edits in more complex settings, including in vivo, analogous to conditional systems that allow for tissue or time-specific gene knockouts.

### REFERENCES

1. Anzalone, A. V., Koblan, L. W. & Liu, D. R. Genome editing with CRISPR-Cas nucleases, base editors, transposases and prime editors. Nat. Biotechnol. 38, 824-844 (2020).
2. Rees, H. A. & Liu, D. R. Base editing: precision chemistry on the genome and transcriptome of living cells. Nat. Rev. Genet. 19, 770-788 (2018).
3. Jinek, M. et al. A programmable dual-RNA-guided DNA endonuclease in adaptive bacterial immunity. Science 337, 816-821 (2012).
4. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
5. Gaudelli, N. M. et al. Programmable base editing of A*T to G*C in genomic DNA without DNA cleavage. Nature 551, 464-471 (2017).
6. Komor, A. C. et al. Improved base excision repair inhibition and bacteriophage Mu Gam protein yields C:G-to-T:A base editors with higher efficiency and product purity. Sci. Adv. 3, eaao4774 (2017).
7. Liu, L. D. et al. Intrinsic Nucleotide Preference of Diversifying Base Editors Guides Antibody Ex Vivo Affinity Maturation. Cell. Rep. 25, 884-892.e3 (2018).
8. Ma, Y. et al. Targeted AID-mediated mutagenesis (TAM) enables efficient genomic diversification in mammalian cells. Nat. Methods 13, 1029-1035 (2016).
9. Green, A. M. & Weitzman, M. D. The spectrum of APOBEC3 activity: From anti-viral agents to anti-cancer opportunities. DNA Repair (Amst) 83, 102700 (2019).
10. Feng, Y., Seija, N., D I Noia, J. M. & Martin, A. AID in Antibody Diversification: There and Back Again. Trends Immunol. 41, 586-600 (2020).
11. Liu, M. & Schatz, D. G. Balancing AID and DNA repair during somatic hypermutation. Trends Immunol. 30, 173-181 (2009).
12. Siriwardena, S. U., Chen, K. & Bhagwat, A. S. Functions and Malfunctions of Mammalian DNA-Cytosine Deaminases. Chem. Rev. 116, 12688-12710 (2016).
13. Burns, M. B. et al. APOBEC3B is an enzymatic source of mutation in breast cancer. Nature 494, 366-370 (2013).
14. Chiarle, R. et al. Genome-wide translocation sequencing reveals mechanisms of chromosome breaks and rearrangements in B cells. Cell 147, 107-119 (2011).
15. Robbiani, D. F. & Nussenzweig, M. C. Chromosome translocation, B cell lymphoma, and activation-induced cytidine deaminase. Annu. Rev. Pathol. 8, 79-103 (2013).
16. Burns, M. B., Temiz, N. A. & Harris, R. S. Evidence for APOBEC3B mutagenesis in multiple human cancers. Nat. Genet. (2013).
17. Roberts, S. A. et al. An APOBEC cytidine deaminase mutagenesis pattern is widespread in human cancers. Nat. Genet. 45, 970-976 (2013).
18. Kim, D. et al. Genome-wide target specificities of CRISPR RNA-guided programmable deaminases. Nat. Biotechnol. 35, 475-480 (2017).
19. Zuo, E. et al. Cytosine base editor generates substantial off-target single-nucleotide variants in mouse embryos. Science 364, 289-292 (2019).
20. Zhou, C. et al. Off-target RNA mutation induced by DNA base editing and its elimination by mutagenesis. Nature 571, 275-278 (2019).
21. Kim, D., Kim, D. E., Lee, G., Cho, S. I. & Kim, J. S. Genome-wide target specificity of CRISPR RNA-guided adenine base editors. Nat. Biotechnol. 37, 430-435 (2019).
22. Grunewald, J. et al. Transcriptome-wide off-target RNA editing induced by CRISPR-guided DNA base editors. Nature 569, 433-437 (2019).
23. Jin, S. et al. Cytosine, but not adenine, base editors induce genome-wide off-target mutations in rice. Science 364, 292-295 (2019).
24. Gangopadhyay, S. A. et al. Precision Control of CRISPR-Cas9 Using Small Molecules and Light. Biochemistry 58, 234-244 (2019).
25. Grunewald, J. et al. CRISPR DNA base editors with reduced RNA off-target and self-editing activities. Nat. Biotechnol. (2019).
26. Rees, H. A., Wilson, C., Doman, J. L. & Liu, D. R. Analysis and minimization of cellular RNA editing by DNA adenine base editors. Sci. Adv. 5, eaax5717 (2019).
27. Iyer, L. M., Zhang, D., Rogozin, I. B. & Aravind, L. Evolution of the deaminase fold and multiple origins of eukaryotic editing and mutagenic nucleic acid deaminases from bacterial toxin systems. Nucleic Acids Res. 39, 9473-9497 (2011).
28. Ear, P. H. & Michnick, S. W. A general life-death selection strategy for dissecting protein functions. Nat. Methods 6, 813-816 (2009).
29. Qiao, Q. et al. AID Recognizes Structured DNA for Class Switch Recombination. Mol. Cell 67, 361-373.e4 (2017).
30. Gajula, K. S. et al. High-throughput mutagenesis reveals functional determinants for DNA targeting by activation-induced deaminase. Nucleic Acids Res. 42, 9964-9975 (2014).
31. Wang, M., Yang, Z., Rada, C. & Neuberger, M. S. AID upmutants isolated using a high-throughput screen highlight the immunity/cancer balance limiting DNA deaminase activity. Nat. Struct. Mol. Biol. 16, 769-776 (2009).
32. Cabantous, S., Terwilliger, T. C. & Waldo, G. S. Protein tagging and detection with engineered self-assembling fragments of green fluorescent protein. Nat. Biotechnol. 23, 102-107 (2005).
33. Kohli, R. M. et al. A portable hotspot recognition loop transfers sequence preferences from APOBEC family members to activation-induced cytidine deaminase. J. Biol. Chem. 284, 22898-22904 (2009).
34. Wang, M., Rada, C. & Neuberger, M. S. A high-throughput assay for DNA deaminases. Methods Mol. Biol. 718, 171-184 (2011).
35. Zong, Y. et al. Efficient C-to-T base editing in plants using a fusion of nCas9 and human APOBEC3A. Nat. Biotechnol. (2018).
36. Gehrke, J. M. et al. An APOBEC3A-Cas9 base editor with minimized bystander and off-target activities. Nat. Biotechnol. 36, 977-982 (2018).
37. Landry, S., Narvaiza, I., Linfesty, D. C. & Weitzman, M. D. APOBEC3A can activate the DNA damage response and cause cell-cycle arrest. EMBO Rep. 12, 444-450 (2011).
38. Voβ, S., Klewer, L. & Wu, Y. W. Chemically induced dimerization: reversible and spatiotemporal control of protein function in cells. Curr. Opin. Chem. Biol. 28, 194-201 (2015).
39. Koblan, L. W. et al. Improving cytidine and adenine base editors by expression optimization and ancestral reconstruction. Nat. Biotechnol. 36, 843-846 (2018).
40. Thuronyi, B. W. et al. Continuous evolution of base editors with expanded target compatibility and improved activity. Nat. Biotechnol. (2019).
41. Mak, C. H., Pham, P., Afif, S. A. & Goodman, M. F. A mathematical model for scanning and catalysis on single-stranded DNA, illustrated with activation-induced deoxycytidine deaminase. J. Biol. Chem. 288, 29786-29795 (2013).
42. Tsai, S. Q. et al. GUIDE-seq enables genome-wide profiling of off-target cleavage by CRISPR-Cas nucleases. Nat. Biotechnol. 33, 187-197 (2015).
43. Mok, B. Y. et al. A bacterial cytidine deaminase toxin enables CRISPR-free mitochondrial base editing. Nature 583, 631-637 (2020).
44. Tsai, S. Q. et al. Dimeric CRISPR RNA-guided FokI nucleases for highly specific genome editing. Nat. Biotechnol. 32, 569-576 (2014).
45. Schutsky, E. K. et al. Nondestructive, base-resolution sequencing of 5-hydroxymethylcytosine using a DNA deaminase. Nat. Biotech. 36, 1083-1090 (2018).
46. Schutsky, E. K., Nabel, C. S., Davis, A. K. F., DeNizio, J. E. & Kohli, R. M. APOBEC3A efficiently deaminates methylated, but not TET-oxidized, cytosine bases in DNA. Nucleic Acids Res. 45, 7655-7665 (2017).
47. Lee, M. et al. Engineered Split-TET2 Enzyme for Inducible Epigenetic Remodeling. J. Am. Chem. Soc. 139, 4659-4662 (2017).
48. Xu, Y. et al. A TFIID-SAGA Perturbation that Targets MYB and Suppresses Acute Myeloid Leukemia. Cancer. Cell. 33, 13-28.e8 (2018).
49. Tarumoto, Y. et al. LKB1, Salt-Inducible Kinases, and MEF2C Are Linked Dependencies in Acute Myeloid Leukemia. Mol. Cell 69, 1017-1027.e6 (2018).
50. Clement, K. et al. CRISPResso2 provides accurate and rapid genome editing sequence analysis. Nat. Biotechnol. 37, 224-226 (2019).
51. Wang, X. et al. Cas12a Base Editors Induce Efficient and Specific Editing with Low DNA Damage Response. Cell. Rep. 31, 107723 (2020).

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A first fusion protein for precise control of targeted base editing in a nucleic acid of interest, comprising an optional accessory module, a targeting module, a first portion of a split deaminase operably linked to a first member of a specific binding pair, and a second fusion protein comprising a second portion of a split deaminase operably linked to a second member of a specific binding pair, said specific binding pair members dimerizing upon contact with a dimerization agent or spontaneously; each of said first and second fusion proteins lacking deaminase activity until reformed,
wherein dimerization causes two portions of said split deaminase enzyme to reform thereby resulting in formation of a functional base editor complex which edits a site of interest on a nucleic acid bound by the targeting module.

2. A first fusion protein for precise control of targeted base editing in a nucleic acid of interest comprising, a first portion of a split deaminase, operably linked to a first portion of a split targeting module, said targeting module being operably linked to a first member of a specific binding pair, and a second fusion protein comprising a second portion of a split deaminase operably linked to a second portion of a split targeting module operably linked to a second specific binding pair member, said specific binding pair members dimerizing upon contact with a dimerization agent or dimerizing spontaneously, each of said first and second fusion proteins lacking deaminase activity until reformed,
wherein dimerization causes two portions of said split deaminase enzyme and said targeting module to reform thereby resulting in formation of functional base editor complex which edits a site of interest on a nucleic acid bound by the targeting module.

3. A reformed deaminase enzyme fusion protein, comprising a first fusion protein with the first portion of a split deaminase operably linked to a first member of a specific binding pair, and a second fusion protein comprising a second portion of a split deaminase operably linked to a second member of a specific binding pair, each of said first and second fusion proteins lacking deaminase activity until reformed, said binding pairs members dimerizing upon contact with a dimerization agent or dimerizing spontaneously, wherein dimerization reforms said split deaminase enzyme restoring activity thereto.

4. The fusion protein of claim 1, wherein said targeting molecule is selected from nCas9, dCas9, dCas12, nCas12, xCas9, Cas13, transcription activator effector-like effectors (TALENs), and zinc finger nucleases (ZFNs), said targeting module comprising a sequence which directs said base editing complex to the site to be edited and optionally being split.

5. The fusion protein of claim 1, wherein said deaminase protein is selected from rat or human APOBEC1, human APOBEC3A, APOBEC3B, APOBEC3C, APOBEC3DE, APOBEC3F, APOBEC3G, Activation-induced cytidine deaminase (AID), CDA from lamprey, mutant version of Adenosine Deaminases (TadA) engineered to act on DNA, and Adenosine Deaminase acting on dsRNA (ADAR) or proteins having at least 90% identity with said deaminase protein.

6. The fusion protein of claim 1, wherein said accessory molecule is present and selected from the group consisting of UGI, 2x UGI, and μ-GAM.

7. The fusion protein of claim 1, present in a cell comprising a dimerization agent.

8. The fusion protein of claim 1, wherein said first and second specific binding pairs are selected from
a) FKBP and FRB wherein binding is induced by contact with dimerization agent rapamycin or a rapamycin analog,
b) FKBP-F36V and FKBP-F36V wherein binding is induced by dimerization agent AP1903, and
c) BCLxl and scAZI, where binding is induced with dimerization agent ABT737, and CRY2 and CIB1 where binding is induced by light.

9. The fusion protein of claim 1, wherein an internal ribosome entry sequence (IRES) separates the two split fragments causing expression of two independently translated split protein fragments which do not require further protease processing.

10. The fusion protein of claim 1, wherein said first and second binding pairs members are GFP 1-10 and GFP11 which dimerize spontaneously.

11. The fusion protein of claim 1, wherein said nucleic acid to be edited is DNA or RNA.

12. (canceled)

13. A method of deaminating one or more selected bases in a target nucleic acid comprising contacting the target nucleic acid with the fusion protein and dimerization agent of claim 1, wherein said base is a cytosine or an adenosine.

14. (canceled)

15. An isolated host cell comprising the fusion protein of claim 1.

16. (canceled)

17. One or more nucleic acids encoding a fusion proteins said fusion protein comprising
i) a targeting module which directs said base editing complex to the site to be edited, a first portion of a split deaminase operably linked to a first member of a specific binding pair, and a second fusion protein comprising a second portion of a split deaminase operably linked to a second member of a specific binding pair, and optionally an accessory module, said specific binding pair members dimerizing upon contact with a dimerization agent or which dimerizing spontaneously;
ii) a first portion of a split deaminase, operably linked to a first portion of a split targeting module, said targeting module being operably linked to a first member of a specific binding pair, and a second fusion protein comprising a second portion of a split deaminase operably linked to a second portion of a split targeting module operably linked to a second specific binding pair member, said specific binding pair members dimerizing upon contact with a dimerization agent or dimerizing spontaneously;
or
iii) a first fusion protein with the first portion of a split deaminase operably linked to a first member of a specific binding pair, and a second fusion protein comprising a second portion of a split deaminase operably linked to a second member of a specific binding pair, each of said first and second fusion proteins lacking deaminase activity until reformed, said binding pairs members dimerizing upon contact with a dimerization agent or dimerizing spontaneously, wherein dimerization reforms said split deaminase enzyme restoring activity thereto;
wherein dimerization causes two portions of a split deaminase enzyme of i) or ii) to reform thereby resulting in formation of a functional base editor complex which edits a site of interest on a nucleic acid bound by the targeting module or
wherein dimerization of said first and second fusion proteins of iii) reforms said split deaminase enzyme restoring activity thereto.

18. At least one expression vector comprising at least one nucleic acid encoding at least one fusion protein of claim 17.

19. An expression vector as claimed in claim 18, comprising a construct shown in FIG. 13.

20. The expression vector of claim 18, selected from the group consisting of a retroviral vector, an adenoviral vector, an adeno-associated viral vector, a lentiviral vector, and a plasmid vector.

21. The nucleic acid of claim 17, which is a DNA or an RNA RNA.

22. A composition comprising the expression vector of claim 18, further comprising one or more of a liposome, a nanoparticle, a pharmaceutically acceptable carrier, and a buffer.

23. A method of deaminating one or more selected bases in a target nucleic acid comprising contacting a cell harboring the target nucleic acid with the nucleic acid of claim 17 under conditions where said fusion proteins are expressed, and optionally a dimerization agent, thereby deaminating said base in said target nucleic acid.

24. A method for producing a reformed active deaminase enzyme of claim 3 for deaminating a target nucleic acid, comprising incubating said first and second fusion proteins and optionally a dimerization agent under conditions where binding between said operably linked specific binding pair members reforms active deaminase enzyme.

25. A kit for practicing the methods of claim 13.

26. The fusion protein of claim 2, wherein
said targeting molecule is selected from nCas9, dCas9, dCas12, nCas12, xCas9, Cas13, transcription activator effector-like effectors (TALENs), and zinc finger nucleases (ZFNs), said targeting module comprising a sequence which directs said base editing complex to the site to be edited and optionally being split,
said deaminase protein is selected from rat or human APOBEC1, human APOBEC3A, APOBEC3B, APOBEC3C, APOBEC3DE, APOBEC3F, APOBEC3G, Activation-induced cytidine deaminase (AID), CDA from lamprey, mutant version of Adenosine Deaminases (TadA) engineered to act on DNA, and Adenosine Deaminase acting on dsRNA (ADAR) or proteins having at least 90% identity with said deaminase protein;
said base is a cytosine or an adenosine; and
said first and second specific binding pairs are selected from
a) FKBP and FRB wherein binding is induced by contact with dimerization agent rapamycin or a rapamycin analog,
b) FKBP-F36V and FKBP-F36V wherein binding is induced by dimerization agent AP1903, and
c) BCLxl and scAZI, where binding is induced with dimerization agent ABT737, and CRY2 and CIB1 where binding is induced by light; and
d) GFP 1-10 and GFP11 which dimerize spontaneously.

27. The fusion protein of claim 3, wherein
said deaminase protein is selected from rat or human APOBEC1, human APOBEC3A, APOBEC3B, APOBEC3C, APOBEC3DE, APOBEC3F, APOBEC3G, Activation-induced cytidine deaminase (AID), CDA from lamprey, mutant version of Adenosine Deaminases (TadA) engineered to act on DNA, and Adenosine Deaminase acting on dsRNA (ADAR) or proteins having at least 90% identity with said deaminase protein;
said base is a cytosine or an adenosine; and
said first and second specific binding pairs are selected from
a) FKBP and FRB wherein binding is induced by contact with dimerization agent rapamycin or a rapamycin analog,
b) FKBP-F36V and FKBP-F36V wherein binding is induced by dimerization agent AP1903, and
c) BCLxl and scAZI, where binding is induced with dimerization agent ABT737, and CRY2 and CIB1 where binding is induced by light; and
d) GFP 1-10 and GFP11 which dimerize spontaneously.

28. A method of deaminating one or more selected bases in a target nucleic acid comprising contacting the target nucleic acid with the fusion protein and optionally a dimerization agent of claim 26.

29. A method of deaminating one or more selected bases in a target nucleic acid comprising contacting the target nucleic acid with the fusion protein and optionally a dimerization agent of claim 27.


SEQUENCE LISTING (58 sequences)

SEQ ID NO: 1 - protein, 4 aa, Artificial Sequence
  Linker
GGSG

SEQ ID NO: 2 - protein, 4 aa, Artificial Sequence
  Linker
GGGS

SEQ ID NO: 3 - protein, 5 aa, Artificial Sequence
  Linker
GGGGS

SEQ ID NO: 4 - protein, 13 aa, Artificial Sequence
  Linker
GGGSGGGGSGGGS

SEQ ID NO: 5 - DNA, 25 bp, Artificial Sequence
  gRNA_KNB1_top
caccgcaagcagaagaacggcatca

SEQ ID NO: 6 - DNA, 25 bp, Artificial Sequence
  gRNA_KNB1_bottom
aaactgatgccgttcttctgcttgc

SEQ ID NO: 7 - DNA, 26 bp, Artificial Sequence
  XmaI ABEmax Forward
ctgagacacccgggacaagcgagagc

SEQ ID NO: 8 - DNA, 18 bp, Artificial Sequence
  XmaI ABEmax Reverse
agccagaggagcctccgc

SEQ ID NO: 9 - DNA, 25 bp, Artificial Sequence
  XmaI BE4max Forward
gcgagacacccgggacaagcgagtc

SEQ ID NO: 10 - DNA, 18 bp, Artificial Sequence
  XmaI BE4max Reverse
tgccagaggatcctccgc

SEQ ID NO: 11 - DNA, 88 bp, Artificial Sequence
  splitCD FRB/FKBP Forward
gtcagatccgctagagatccgcggccgctaatacgactcactatagggagagccgccaccatggaacaaaaacttatttctgaagaag

SEQ ID NO: 12 - DNA, 61 bp, Artificial Sequence
  AIDC12 FRB/FKBP BE3 Reverse
gtgtggcggactctgaggtcccgggagtctcgctgccgctcagcagaatacgacgcagctg

SEQ ID NO: 13 - DNA, 62 bp, Artificial Sequence
  A3A FRB/FKBP BE3 Reverse
tgtggcggactctgaggtcccgggagtctcgctgccgctgtttccctgattctggagaatgg

SEQ ID NO: 14 - DNA, 56 bp, Artificial Sequence
  evorA1 FRB/FKBP BE3 Reverse
tgtggcggactctgaggtcccgggagtctcgctgccgctcttcaggcctgtggccc

SEQ ID NO: 15 - DNA, 88 bp, Artificial Sequence
  AIDC12 FRB/FKBP BE4max Reverse
ctggtgttgctgactcgcttgtcccgggtgtctcgctgccagaggatcctccgctagatccgccagacagcagaatacgacgcagctg

SEQ ID NO: 16 - DNA, 90 bp, Artificial Sequence
  A3A FRB/FKBP BE4max Reverse
ctggtgttgctgactcgcttgtcccgggtgtctcgctgccagaggatcctccgctagatccgccagagtttccctgattctggagaatgg

SEQ ID NO: 17 - DNA, 83 bp, Artificial Sequence
  evorA1 FRB/FKBP BE4max Reverse
ctggtgttgctgactcgcttgtcccgggtgtctcgctgccagaggatcctccgctagatccgccagacttcaggcctgtggcc

SEQ ID NO: 18 - DNA, 81 bp, Artificial Sequence
  monoABEmax Reverse
gtgttgcgctctcgcttgtcccgggtgtctcagagccagaggagcctccgctagatcctccggagtcggtggagctctggg

SEQ ID NO: 19 - DNA, 1536 bp, Artificial Sequence
  Myc-NLS-A3An-FRB-T2A-FKBP12-A3Ac-FlagTag
atggaacaaaaacttatttctgaagaagatctgaaaaggccggcggccacgaaaaaggccggccaggcaaaaaagaaaaagggaggttccgctagcggaggttcgatggaagccagcccagcatccgggcccagacacttgatggatccacacatattcacttccaactttaacaatggcattggaaggcataagacctacctgtgctacgaagtggagcgcctggacaatggcacctcggtcaagatggaccagcacaggggctttctacacaaccaggctaagaatcttctctgtggcttttacggccgccatgcggagctgcgcttcttggacctggttccttctttgcagttggacccgggcgcgccgggaggtggtggcagcggtggaggaggttctgggggcggtggctcaattttatggcatgagatgtggcatgagggtttggaagaggcatctagattgtatttcggtgaaagaaatgtcaagggaatgttcgaagttttagaaccgttgcacgctatgatggagagaggtccacagactctaaaggagacttccttcaaccaagcttatggaagggacctaatggaggctcaagaatggtgtagaaaatacatgaaaagtggaaatgtaaaggaccttacacaagcttgggatctctactaccatgtttttaggagaatatctaaaggaagtggtgagggtaggggaagtttattaacctgtggggatgttgaagaaaatccaggtcctatgggcgtacaagttgaaactatcagccctggggacggcagaacctttccgaagaggggacagacatgtgttgttcactatactggaatgttggaagatggtaagaagttcgatagcagcagagataggaataaaccatttaaattcatgcttggcaagcaagaagtgattaggggttgggaagaaggtgtcgctcaaatgagtgtaggtcagagggctaagttaacaattagtcctgattatgcttatggcgctacaggtcatccaggaatcattcccccacatgctactcttgttttcgacgttgaattgcttaagcttgaaggatcaggttctggatctggttcaggatcaggctcacccgggcttgcccagatctacagggtcacctggttcatctcctggagcccctgcttctcctggggctgtgccggggaagtgcgtgcgttcctgcaggagaacacacacgtgagactgcgtatcttcgctgcccgcatctatgattacgaccccctatataaggaggcactgcaaatgctgcgggatgctggggcccaagtctccatcatgacctacgatgaatttaagcactgctgggacacctttgtggaccaccagggatgtcccttccagccctgggatggactagatgagcacagccaagccctgagtgggaggctgcgggccattctccagaatcagggaaacggtaccgggtcgggtagtggctctggtagtggttctggttctgattacaaagacgatgacgataagtaa

SEQ ID NO: 20 - DNA, 1482 bp, Artificial Sequence
  Myc-NLS-AID′n-FRB-T2A-FKBP12-AID′-FlagTag
atggaacaaaaacttatttctgaagaagatctgaaaaggccggcggccacgaaaaaggccggccaggcaaaaaagaaaaagggaggttccgctagcggaggttcgatggatagcctgctgatgaaccgtcgtgaatttctgtatcagtttaaaaacgtgcgttgggcgaaaggccgtcgtgaaacctatctgtgctatgtggtgaaacgtcgtgatagcgcgaccagctttagcctggattttggctatctgcgtaacaaaaacggctgccatgtggaactgctgtttctgcgttatattagcgattgggatctggatccgggcgcgccgggaggtggtggcagcggtggaggaggttctgggggcggtggctcaattttatggcatgagatgtggcatgagggtttggaagaggcatctagattgtatttcggtgaaagaaatgtcaagggaatgttcgaagttttagaaccgttgcacgctatgatggagagaggtccacagactctaaaggagacttccttcaaccaagcttatggaagggacctaatggaggctcaagaatggtgtagaaaatacatgaaaagtggaaatgtaaaggaccttacacaagcttgggatctctactaccatgtttttaggagaatatctaaaggaagtggtgagggtaggggaagtttattaacctgtggggatgttgaagaaaatccaggtcctatgggcgtacaagttgaaactatcagccctggggacggcagaacctttccgaagaggggacagacatgtgttgttcactatactggaatgttggaagatggtaagaagttcgatagcagcagagataggaataaaccatttaaattcatgcttggcaagcaagaagtgattaggggttgggaagaaggtgtcgctcaaatgagtgtaggtcagagggctaagttaacaattagtcctgattatgcttatggcgctacaggtcatccaggaatcattcccccacatgctactcttgttttcgacgttgaattgcttaagcttgaaggatcaggttctggatctggttcaggatcaggctcacccgggcttggccgttgctatcgtgtgacctggtttatcagctggagcccgtgctatgattgcgcgcgtcatgtggcggattttctgcgtggcaacccgaacctgagcctgcgtatttttaccgcgcgtctgtatttttgcgaagccggcaggcgtgaaccggaaggcctgcgtcgtctgcatcgtgcgggcgtgcagattgcgattatgacctttaaagattatttttattgctggaacacctttgtggaaaaccatggacgtacctttaaagcgtgggaaggcctgcatgaaaacagcgtgcgtctgagccgtcagctgcgtcgtattctgctgggtaccgggtcgggtagtggctctggtagtggttctggttctgattacaaagacgatgacgataagtaa

SEQ ID NO: 21 - DNA, 1557 bp, Artificial Sequence
  Myc-NLS-evorA1n-FRB-T2A-FKBP12-evorA1c
atggaacaaaaacttatttctgaagaagatctgaaaaggccggcggccacgaaaaaggccggccaggcaaaaaagaaaaagggaggttccgctagcggaggttcgatgagttcaaagactgggcctgtcgccgtcgatccaaccctgcgccgccggattgaacctcacgagtttgaagtgttctttgacccccgggagctgagaaaggagacatgcctgctgtacgagatcaactggggaggcaggcactccatctggaggcacacctctcagaacacaaataagcacgtggaggtgaacttcatcgagaagtttaccacagagcggtacttctgccccggcgcgccgggaggtggtggcagcggtggaggaggttctgggggcggtggctcaattttatggcatgagatgtggcatgagggtttggaagaggcatctagattgtatttcggtgaaagaaatgtcaagggaatgttcgaagttttagaaccgttgcacgctatgatggagagaggtccacagactctaaaggagacttccttcaaccaagcttatggaagggacctaatggaggctcaagaatggtgtagaaaatacatgaaaagtggaaatgtaaaggaccttacacaagcttgggatctctactaccatgtttttaggagaatatctaaaggaagtggtgagggtaggggaagtttattaacctgtggggatgttgaagaaaatccaggtcctatgggcgtacaagttgaaactatcagccctggggacggcagaacctttccgaagaggggacagacatgtgttgttcactatactggaatgttggaagatggtaagaagttcgatagcagcagagataggaataaaccatttaaattcatgcttggcaagcaagaagtgattaggggttgggaagaaggtgtcgctcaaatgagtgtaggtcagagggctaagttaacaattagtcctgattatgcttatggcgctacaggtcatccaggaatcattcccccacatgctactcttgttttcgacgttgaattgcttaagcttgaaggatcaggttctggatctggttcaggatcaggctcacccgggcttaataccagatgtagcatcacatggtttctgagctggtccccttgcggagagtgtagcagggccatcaccgagttcctgtccagatatccaaatgtgacactgtttatctacatcgccaggctgtatcacctggcaaacccaaggaataggcagggcctgcgcgatctgatcagctccggcgtgaccatccagatcatgacagagcaggagtccggctactgctggcacaacttcgtgaattattctcctagcaacgagtcccactggcctaggtacccacacctgtgggtgcgcctgtacgtgctggagctgtattgcatcatcctgggcctgcccccttgtctgaatatcctgcggagaaagcagagccagctgacctcctttacaatcgccctgcagtcttgtcactatcagaggctgccaccccacatcctgtgggccacaggcctgaag

SEQ ID NO: 22 - DNA, 1440 bp, Artificial Sequence
  Myc-NLS-TadAn-FRB-T2A-FKBP12-TadAc
atggaacaaaaacttatttctgaagaagatctgaaaaggccggcggccacgaaaaaggccggccaggcaaaaaagaaaaagggaggttccgctagcggaggttcgatgtctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgggcgcgccgggaggtggtggcagcggtggaggaggttctgggggcggtggctcaattttatggcatgagatgtggcatgagggtttggaagaggcatctagattgtatttcggtgaaagaaatgtcaagggaatgttcgaagttttagaaccgttgcacgctatgatggagagaggtccacagactctaaaggagacttccttcaaccaagcttatggaagggacctaatggaggctcaagaatggtgtagaaaatacatgaaaagtggaaatgtaaaggaccttacacaagcttgggatctctactaccatgtttttaggagaatatctaaaggaagtggtgagggtaggggaagtttattaacctgtggggatgttgaagaaaatccaggtcctatgggcgtacaagttgaaactatcagccctggggacggcagaacctttccgaagaggggacagacatgtgttgttcactatactggaatgttggaagatggtaagaagttcgatagcagcagagataggaataaaccatttaaattcatgcttggcaagcaagaagtgattaggggttgggaagaaggtgtcgctcaaatgagtgtaggtcagagggctaagttaacaattagtcctgattatgcttatggcgctacaggtcatccaggaatcattcccccacatgctactcttgttttcgacgttgaattgcttaagcttgaaggatcaggttctggatctggttcaggatcaggctcacccgggcttattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgacggtaccgggtcgggtagtggctctggtagtggttctggttctgattacaaagacgatgacgataagtaa

SEQ ID NO: 23 - DNA, 19 bp, Artificial Sequence
  d2GFP forward primer 1
cttcaaggaggacggcaac

SEQ ID NO: 24 - DNA, 15 bp, Artificial Sequence
  d2GFP reverse primer 1
gtggtcggcgagctg

SEQ ID NO: 25 - DNA, 846 bp, Artificial Sequence
  d2GFP sequence
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtgtag

SEQ ID NO: 26 - DNA, 777 bp, Artificial Sequence
  Linker-GFP-Linker sequence
ggatccgctggctccgctgctggttctggcgaattcatgagcaaaggagaagaacttttcactggagttgtcccaattcttgttgaattagatggtgatgttaatgggcacaaattttctgtcagaggagagggtgaaggtgatgctacaatcggaaaactcacccttaaatttatttgcactactggaaaactacctgttccatggccaacacttgtcactactctgacctatggtgttcaatgcttttcccgttatccggatcacatgaaaaggcatgactttttcaagagtgccatgcccgaaggttatgtacaggaacgcactatatctttcaaagatgacgggaaatacaagacgcgtgctgtagtcaagtttgaaggtgatacccttgttaatcgtatcgagttaaagggtactgattttaaagaagatggaaacattctcggacacaaactcgagtacaactttaactcacacaatgtatacatcacggcagacaaacaaaagaatggaatcaaagctaacttcacagttcgccacaacgttgaagatggttccgttcaactagcagaccattatcaacaaaatactccaattggcgatggccctgtccttttaccagacaaccattacctgtcaacacaaactgtcctttcgaaagatcccaacgaaaagggtacccgtgaccacatggtccttcatgagtctgtaaatgctgctgggattacaggtggaggaggttctggaggcggtggaagtggtggcggaggtagc

SEQ ID NO: 27 - DNA, 44 bp, Artificial Sequence
  AIDn Forward
tcgaaaacctgtattttcaggggtcgacaatggatagcctgctg

SEQ ID NO: 28 - DNA, 30 bp, Artificial Sequence
  AIDn Reverse
cagcggatcccggatccagatcccaatcgc

SEQ ID NO: 29 - DNA, 28 bp, Artificial Sequence
  AIDc Forward
cggaggtagcggccgttgctatcgtgtg

SEQ ID NO: 30 - DNA, 39 bp, Artificial Sequence
  AIDc Reverse
gggctttgtttagcagcctaggctacaggcccagggtac

SEQ ID NO: 31 - DNA, 27 bp, Artificial Sequence
  Linker-GFP-Linker B1/B2 Forward
tctggatccgggatccgctggctccgc

SEQ ID NO: 32 - DNA, 30 bp, Artificial Sequence
  Linker-GFP-Linker B1/B2 Reverse
agcaacggccgctacctccgccaccacttc

SEQ ID NO: 33 - DNA, 26 bp, Artificial Sequence
  AIDn Forward
cagaattcgaaaacctgtattttcag

SEQ ID NO: 34 - DNA, 23 bp, Artificial Sequence
  AIDc Reverse
ctttcgggctttgtttagcagcc

SEQ ID NO: 35 - DNA, 6770 bp, Artificial Sequence
  AID* pET41
taatacgactcactataggggaattgtgagcggataacaattcccctctagaaataattttgtttaactttaagaaggagatatacatatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcgaaaacctgtattttcaggggtcgacaatggatagcctgctgatgaaccgtcgtgaatttctgtatcagtttaaaaacgtgcgttgggcgaaaggccgtcgtgaaacctatctgtgctatgtggtgaaacgtcgtgatagcgcgaccagcgaaagcctggattttggctatctgcgtaacaaaaacggctgccatgtggaactgctgtttctgcgttatattagcgattgggatctggatccgggccgttgctatcgtgtgacctggtttatcagctggagcccgtgctatgattgcgcgcgtcatgtggcggattttctgcgtggcaacccgaacctgagcctgcgtatttttaccgcgcgtctgtatttttgcgaagccggcaggcgtgaaccggaaggcctgcgtcgtctggcggaagcgggcgtgcagattgcgattatgacctataaagattatgaatattgctggaacacctttgtggaaaaccatggacgtacctttaaagcgtgggaaggcctgcatgaaaacagcgtgcgtctgagccgtcagctgcgtcgtattctgctgccgctgtatgaagtggatgatctgcgtgatgcgtttcgtaccctgggcctgtagcctaggctgctaaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaa

SEQ ID NO: 36 - DNA, 7547 bp, Artificial Sequence
  AID*-INS1 pET41
taatacgactcactataggggaattgtgagcggataacaattcccctctagaaataattttgtttaactttaagaaggagatatacatatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcgaaaacctgtattttcaggggtcgacaatggatagcctgctgatgaaccgtcgtgaatttctgtatcagtttaaaaacgtgcgttgggcgaaaggccgtcgtgaaacctatctgtgctatgtggtgaaacgtcgtgatagcgcgaccggatccgctggctccgctgctggttctggcgaattcatgagcaaaggagaagaacttttcactggagttgtcccaattcttgttgaattagatggtgatgttaatgggcacaaattttctgtcagaggagagggtgaaggtgatgctacaatcggaaaactcacccttaaatttatttgcactactggaaaactacctgttccatggccaacacttgtcactactctgacctatggtgttcaatgcttttcccgttatccggatcacatgaaaaggcatgactttttcaagagtgccatgcccgaaggttatgtacaggaacgcactatatctttcaaagatgacgggaaatacaagacgcgtgctgtagtcaagtttgaaggtgatacccttgttaatcgtatcgagttaaagggtactgattttaaagaagatggaaacattctcggacacaaactcgagtacaactttaactcacacaatgtatacatcacggcagacaaacaaaagaatggaatcaaagctaacttcacagttcgccacaacgttgaagatggttccgttcaactagcagaccattatcaacaaaatactccaattggcgatggccctgtccttttaccagacaaccattacctgtcaacacaaactgtcctttcgaaagatcccaacgaaaagggtacccgtgaccacatggtccttcatgagtctgtaaatgctgctgggattacaggtggaggaggttctggaggcggtggaagtggtggcggaggtagcagcgaaagcctggattttggctatctgcgtaacaaaaacggctgccatgtggaactgctgtttctgcgttatattagcgattgggatctggatccgggccgttgctatcgtgtgacctggtttatcagctggagcccgtgctatgattgcgcgcgtcatgtggcggattttctgcgtggcaacccgaacctgagcctgcgtatttttaccgcgcgtctgtatttttgcgaagccggcaggcgtgaaccggaaggcctgcgtcgtctggcggaagcgggcgtgcagattgcgattatgacctataaagattatgaatattgctggaacacctttgtggaaaaccatggacgtacctttaaagcgtgggaaggcctgcatgaaaacagcgtgcgtctgagccgtcagctgcgtcgtattctgctgccgctgtatgaagtggatgatctgcgtgatgcgtttcgtaccctgggcctgtagcctaggctgctaaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaa

SEQ ID NO: 37 - DNA, 7547 bp, Artificial Sequence
  AID*-INS2 pET41
taatacgactcactataggggaattgtgagcggataacaattcccctctagaaataattttgtttaactttaagaaggagatatacatatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcgaaaacctgtattttcaggggtcgacaatggatagcctgctgatgaaccgtcgtgaatttctgtatcagtttaaaaacgtgcgttgggcgaaaggccgtcgtgaaacctatctgtgctatgtggtgaaacgtcgtgatagcgcgaccagcgaaagcctggattttggctatctgcgtaacaaaaacggctgccatgtggaactgctgtttctgcgttatattagcgattgggatctggatccgggatccgctggctccgctgctggttctggcgaattcatgagcaaaggagaagaacttttcactggagttgtcccaattcttgttgaattagatggtgatgttaatgggcacaaattttctgtcagaggagagggtgaaggtgatgctacaatcggaaaactcacccttaaatttatttgcactactggaaaactacctgttccatggccaacacttgtcactactctgacctatggtgttcaatgcttttcccgttatccggatcacatgaaaaggcatgactttttcaagagtgccatgcccgaaggttatgtacaggaacgcactatatctttcaaagatgacgggaaatacaagacgcgtgctgtagtcaagtttgaaggtgatacccttgttaatcgtatcgagttaaagggtactgattttaaagaagatggaaacattctcggacacaaactcgagtacaactttaactcacacaatgtatacatcacggcagacaaacaaaagaatggaatcaaagctaacttcacagttcgccacaacgttgaagatggttccgttcaactagcagaccattatcaacaaaatactccaattggcgatggccctgtccttttaccagacaaccattacctgtcaacacaaactgtcctttcgaaagatcccaacgaaaagggtacccgtgaccacatggtccttcatgagtctgtaaatgctgctgggattacaggtggaggaggttctggaggcggtggaagtggtggcggaggtagcggccgttgctatcgtgtgacctggtttatcagctggagcccgtgctatgattgcgcgcgtcatgtggcggattttctgcgtggcaacccgaacctgagcctgcgtatttttaccgcgcgtctgtatttttgcgaagccggcaggcgtgaaccggaaggcctgcgtcgtctggcggaagcgggcgtgcagattgcgattatgacctataaagattatgaatattgctggaacacctttgtggaaaaccatggacgtacctttaaagcgtgggaaggcctgcatgaaaacagcgtgcgtctgagccgtcagctgcgtcgtattctgctgccgctgtatgaagtggatgatctgcgtgatgcgtttcgtaccctgggcctgtagcctaggctgctaaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaa

SEQ ID NO: 38 - DNA, 7547 bp, Artificial Sequence
  AID*-INS3 pET41
taatacgactcactataggggaattgtgagcggataacaattcccctctagaaataattttgtttaactttaagaaggagatatacatatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcgaaaacctgtattttcaggggtcgacaatggatagcctgctgatgaaccgtcgtgaatttctgtatcagtttaaaaacgtgcgttgggcgaaaggccgtcgtgaaacctatctgtgctatgtggtgaaacgtcgtgatagcgcgaccagcgaaagcctggattttggctatctgcgtaacaaaaacggctgccatgtggaactgctgtttctgcgttatattagcgattgggatctggatccgggccgttgctatcgtgtgacctggtttatcagctggagcccgtgctatgattgcgcgcgtcatgtggcggattttctgcgtggcaacccgggatccgctggctccgctgctggttctggcgaattcatgagcaaaggagaagaacttttcactggagttgtcccaattcttgttgaattagatggtgatgttaatgggcacaaattttctgtcagaggagagggtgaaggtgatgctacaatcggaaaactcacccttaaatttatttgcactactggaaaactacctgttccatggccaacacttgtcactactctgacctatggtgttcaatgcttttcccgttatccggatcacatgaaaaggcatgactttttcaagagtgccatgcccgaaggttatgtacaggaacgcactatatctttcaaagatgacgggaaatacaagacgcgtgctgtagtcaagtttgaaggtgatacccttgttaatcgtatcgagttaaagggtactgattttaaagaagatggaaacattctcggacacaaactcgagtacaactttaactcacacaatgtatacatcacggcagacaaacaaaagaatggaatcaaagctaacttcacagttcgccacaacgttgaagatggttccgttcaactagcagaccattatcaacaaaatactccaattggcgatggccctgtccttttaccagacaaccattacctgtcaacacaaactgtcctttcgaaagatcccaacgaaaagggtacccgtgaccacatggtccttcatgagtctgtaaatgctgctgggattacaggtggaggaggttctggaggcggtggaagtggtggcggaggtagcaacctgagcctgcgtatttttaccgcgcgtctgtatttttgcgaagccggcaggcgtgaaccggaaggcctgcgtcgtctggcggaagcgggcgtgcagattgcgattatgacctataaagattatgaatattgctggaacacctttgtggaaaaccatggacgtacctttaaagcgtgggaaggcctgcatgaaaacagcgtgcgtctgagccgtcagctgcgtcgtattctgctgccgctgtatgaagtggatgatctgcgtgatgcgtttcgtaccctgggcctgtagcctaggctgctaaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaa

SEQ ID NO: 39 - DNA, 7547 bp, Artificial Sequence
  AID*-INS- pET41
taatacgactcactataggggaattgtgagcggataacaattcccctctagaaataattttgtttaactttaagaaggagatatacatatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcgaaaacctgtattttcaggggtcgacaatggatagcctgctgatgaaccgtcgtgaatttctgtatcagtttaaaaacgtgcgttgggcgaaaggccgtcgtgaaacctatctgtgctatgtggtgaaacgtcgtgatagcgcgaccagcgaaagcctggattttggctatctgcgtaacaaaaacggctgccatgtggaactgctgtttctgcgttatattagcgattgggatctggatccgggccgttgctatcgtgtgacctggtttatcagctggagcccgtgctatggatccgctggctccgctgctggttctggcgaattcatgagcaaaggagaagaacttttcactggagttgtcccaattcttgttgaattagatggtgatgttaatgggcacaaattttctgtcagaggagagggtgaaggtgatgctacaatcggaaaactcacccttaaatttatttgcactactggaaaactacctgttccatggccaacacttgtcactactctgacctatggtgttcaatgcttttcccgttatccggatcacatgaaaaggcatgactttttcaagagtgccatgcccgaaggttatgtacaggaacgcactatatctttcaaagatgacgggaaatacaagacgcgtgctgtagtcaagtttgaaggtgatacccttgttaatcgtatcgagttaaagggtactgattttaaagaagatggaaacattctcggacacaaactcgagtacaactttaactcacacaatgtatacatcacggcagacaaacaaaagaatggaatcaaagctaacttcacagttcgccacaacgttgaagatggttccgttcaactagcagaccattatcaacaaaatactccaattggcgatggccctgtccttttaccagacaaccattacctgtcaacacaaactgtcctttcgaaagatcccaacgaaaagggtacccgtgaccacatggtccttcatgagtctgtaaatgctgctgggattacaggtggaggaggttctggaggcggtggaagtggtggcggaggtagcgattgcgcgcgtcatgtggcggattttctgcgtggcaacccgaacctgagcctgcgtatttttaccgcgcgtctgtatttttgcgaagccggcaggcgtgaaccggaaggcctgcgtcgtctggcggaagcgggcgtgcagattgcgattatgacctataaagattatgaatattgctggaacacctttgtggaaaaccatggacgtacctttaaagcgtgggaaggcctgcatgaaaacagcgtgcgtctgagccgtcagctgcgtcgtattctgctgccgctgtatgaagtggatgatctgcgtgatgcgtttcgtaccctgggcctgtagcctaggctgctaaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaa

SEQ ID NO: 40 - DNA, 7547 bp, Artificial Sequence
  AID*-INS+ pET41
taatacgactcactataggggaattgtgagcggataacaattcccctctagaaataattttgtttaactttaagaaggagatatacatatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcgaaaacctgtattttcaggggtcgacaatggatagcctgctgatgaaccgtcgtgaatttctgtatcagtttaaaaacgtgcgttgggcgaaaggccgtcgtgaaacctatctgtgctatgtggtgaaacgtcgtgatagcgcgaccagcgaaagcctggattttggctatctgcgtaacaaaaacggctgccatgtggaactgctgtttctgcgttatattagcgattgggatctggatccgggccgttgctatcgtgtgacctggtttatcagctggagcccgtgctatgattgcgcgcgtcatgtggcggattttctgcgtggcaacccgaacctgagcctgcgtatttttaccgcgcgtctgtatttttgcgaagccggcaggcgtgaaccggaaggcctgcgtcgtctggcggaagcgggcgtgcagattgcgattatgacctataaagattatgaatattgctggaacacctttgtggaaaaccatggacgtacctttaaagcgtgggaaggcctgcatgaaaacagcgtgcgtctgagccgtcagctgcgtcgtattctgctgggatccgctggctccgctgctggttctggcgaattcatgagcaaaggagaagaacttttcactggagttgtcccaattcttgttgaattagatggtgatgttaatgggcacaaattttctgtcagaggagagggtgaaggtgatgctacaatcggaaaactcacccttaaatttatttgcactactggaaaactacctgttccatggccaacacttgtcactactctgacctatggtgttcaatgcttttcccgttatccggatcacatgaaaaggcatgactttttcaagagtgccatgcccgaaggttatgtacaggaacgcactatatctttcaaagatgacgggaaatacaagacgcgtgctgtagtcaagtttgaaggtgatacccttgttaatcgtatcgagttaaagggtactgattttaaagaagatggaaacattctcggacacaaactcgagtacaactttaactcacacaatgtatacatcacggcagacaaacaaaagaatggaatcaaagctaacttcacagttcgccacaacgttgaagatggttccgttcaactagcagaccattatcaacaaaatactccaattggcgatggccctgtccttttaccagacaaccattacctgtcaacacaaactgtcctttcgaaagatcccaacgaaaagggtacccgtgaccacatggtccttcatgagtctgtaaatgctgctgggattacaggtggaggaggttctggaggcggtggaagtggtggcggaggtagcccgctgtatgaagtggatgatctgcgtgatgcgtttcgtaccctgggcctgtagcctaggctgctaaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaa

SEQ ID NO: 41 - DNA, 7655 bp, Artificial Sequence
  AID*-SPL2 pET41
taatacgactcactataggggaattgtgagcggataacaattcccctctagaaataattttgtttaactttaagaaggagatatacatatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcgaaaacctgtattttcaggggtcgacaatggatagcctgctgatgaaccgtcgtgaatttctgtatcagtttaaaaacgtgcgttgggcgaaaggccgtcgtgaaacctatctgtgctatgtggtgaaacgtcgtgatagcgcgaccagcgaaagcctggattttggctatctgcgtaacaaaaacggctgccatgtggaactgctgtttctgcgttatattagcgattgggatctggatccgggatccgctggctccgctgctggttctggcgaattcatgagcaaaggagaagaacttttcactggagttgtcccaattcttgttgaattagatggtgatgttaatgggcacaaattttctgtcagaggagagggtgaaggtgatgctacaatcggaaaactcacccttaaatttatttgcactactggaaaactacctgttccatggccaacacttgtcactactctgacctatggtgttcaatgcttttcccgttatccggatcacatgaaaaggcatgactttttcaagagtgccatgcccgaaggttatgtacaggaacgcactatatctttcaaagatgacgggaaatacaagacgcgtgctgtagtcaagtttgaaggtgatacccttgttaatcgtatcgagttaaagggtactgattttaaagaagatggaaacattctcggacacaaactcgagtacaactttaactcacacaatgtatacatcacggcagacaaacaaaagaatggaatcaaagctaacttcacagttcgccacaacgttgaagatggttccgttcaactagcagaccattatcaacaaaatactccaattggcgatggccctgtccttttaccagacaaccattacctgtcaacacaaactgtcctttcgaaagatcccaacgaaaagggtacctagtcggcggccgcaaaattaatacgactcactataggggaattgtgagcggataacaattccccatcttagtatattagttaagtataagaaggagatatatccatgcgtgaccacatggtccttcatgagtctgtaaatgctgctgggattacaggtggaggaggttctggaggcggtggaagtggtggcggaggtagcggccgttgctatcgtgtgacctggtttatcagctggagcccgtgctatgattgcgcgcgtcatgtggcggattttctgcgtggcaacccgaacctgagcctgcgtatttttaccgcgcgtctgtatttttgcgaagccggcaggcgtgaaccggaaggcctgcgtcgtctggcggaagcgggcgtgcagattgcgattatgacctataaagattatgaatattgctggaacacctttgtggaaaaccatggacgtacctttaaagcgtgggaaggcctgcatgaaaacagcgtgcgtctgagccgtcagctgcgtcgtattctgctgccgctgtatgaagtggatgatctgcgtgatgcgtttcgtaccctgggcctgtagcctaggctgctaaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaa

SEQ ID NO: 42 - DNA, 7096 bp, Artificial Sequence
  AID*-SPL2N pET41
taatacgactcactataggggaattgtgagcggataacaattcccctctagaaataattttgtttaactttaagaaggagatatacatatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcgaaaacctgtattttcaggggtcgacaatggatagcctgctgatgaaccgtcgtgaatttctgtatcagtttaaaaacgtgcgttgggcgaaaggccgtcgtgaaacctatctgtgctatgtggtgaaacgtcgtgatagcgcgaccagcgaaagcctggattttggctatctgcgtaacaaaaacggctgccatgtggaactgctgtttctgcgttatattagcgattgggatctggatccgggatccgctggctccgctgctggttctggcgaattcatgagcaaaggagaagaacttttcactggagttgtcccaattcttgttgaattagatggtgatgttaatgggcacaaattttctgtcagaggagagggtgaaggtgatgctacaatcggaaaactcacccttaaatttatttgcactactggaaaactacctgttccatggccaacacttgtcactactctgacctatggtgttcaatgcttttcccgttatccggatcacatgaaaaggcatgactttttcaagagtgccatgcccgaaggttatgtacaggaacgcactatatctttcaaagatgacgggaaatacaagacgcgtgctgtagtcaagtttgaaggtgatacccttgttaatcgtatcgagttaaagggtactgattttaaagaagatggaaacattctcggacacaaactcgagtacaactttaactcacacaatgtatacatcacggcagacaaacaaaagaatggaatcaaagctaacttcacagttcgccacaacgttgaagatggttccgttcaactagcagaccattatcaacaaaatactccaattggcgatggccctgtccttttaccagacaaccattacctgtcaacacaaactgtcctttcgaaagatcccaacgaaaagggtacctagtcgcaggtacgacgcatcagcctaggctgctaaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaa

SEQ ID NO: 43 - DNA, 5456 bp, Artificial Sequence
  AID*-SPL2C pET41
taatacgactcactataggggaattgtgagcggataacaattcccctctagaaataattttgtttaactttaagaaggagatatacatatgggtacccgtgaccacatggtccttcatgagtctgtaaatgctgctgggattacaggtggaggaggttctggaggcggtggaagtggtggcggaggtagcggccgttgctatcgtgtgacctggtttatcagctggagcccgtgctatgattgcgcgcgtcatgtggcggattttctgcgtggcaacccgaacctgagcctgcgtatttttaccgcgcgtctgtatttttgcgaagccggcaggcgtgaaccggaaggcctgcgtcgtctggcggaagcgggcgtgcagattgcgattatgacctataaagattatgaatattgctggaacacctttgtggaaaaccatggacgtacctttaaagcgtgggaaggcctgcatgaaaacagcgtgcgtctgagccgtcagctgcgtcgtattctgctgccgctgtatgaagtggatgatctgcgtgatgcgtttcgtaccctgggcctgtagcctaggctgctaaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaa

SEQ ID NO: 44 - DNA, 6837 bp, Artificial Sequence
  A3A pET41
taatacgactcactataggggaattgtgagcggataacaattcccctctagaaataattttgtttaactttaagaaggagatatacatatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcgaaaacctgtattttcaggggtcgacaatggaagccagcccagcatccgggcccagacacttgatggatccacacatattcacttccaactttaacaatggcattggaaggcataagacctacctgtgctacgaagtggagcgcctggacaatggcacctcggtcaagatggaccagcacaggggctttctacacaaccaggctaagaatcttctctgtggcttttacggccgccatgcggagctgcgcttcttggacctggttccttctttgcagttggacccggcccagatctacagggtcacttggttcatctcctggagcccctgcttctcctggggctgtgccggggaagtgcgtgcgttccttcaggagaacacacacgtgagactgcgtatcttcgctgcccgcatctatgattacgaccccctatataaggaggcactgcaaatgctgcgggatgctggggcccaagtctccatcatgacctacgatgaatttaagcactgctgggacacctttgtggaccaccagggatgtcccttccagccctgggatggactagatgagcacagccaagccctgagtgggaggctgcgggccattctccagaatcagggaaacggtaccgaaaacctgtattttcagggctcgacaatggatcaccaccaccaccaccaccaccactaacctaggctgctaaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaat

SEQ ID NO: 45 - DNA, 7613 bp, Artificial Sequence
  A3A-INS2 pET41
taatacgactcactataggggaattgtgagcggataacaattcccctctagaaataattttgtttaactttaagaaggagatatacatatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcgaaaacctgtattttcaggggtcgacaatggaagccagcccagcatccgggcccagacacttgatggatccacacatattcacttccaactttaacaatggcattggaaggcataagacctacctgtgctacgaagtggagcgcctggacaatggcacctcggtcaagatggaccagcacaggggctttctacacaaccaggctaagaatcttctctgtggcttttacggccgccatgcggagctgcgcttcttggacctggttccttctttgcagttggacccgggatccgctggctccgctgctggttctggcgaattcatgagcaaaggagaagaacttttcactggagttgtcccaattcttgttgaattagatggtgatgttaatgggcacaaattttctgtcagaggagagggtgaaggtgatgctacaatcggaaaactcacccttaaatttatttgcactactggaaaactacctgttccatggccaacacttgtcactactctgacctatggtgttcaatgcttttcccgttatccggatcacatgaaaaggcatgactttttcaagagtgccatgcccgaaggttatgtacaggaacgcactatatctttcaaagatgacgggaaatacaagacgcgtgctgtagtcaagtttgaaggtgatacccttgttaatcgtatcgagttaaagggtactgattttaaagaagatggaaacattctcggacacaaactcgagtacaactttaactcacacaatgtatacatcacggcagacaaacaaaagaatggaatcaaagctaacttcacagttcgccacaacgttgaagatggttccgttcaactagcagaccattatcaacaaaatactccaattggcgatggccctgtccttttaccagacaaccattacctgtcaacacaaactgtcctttcgaaagatcccaacgaaaagggtacccgtgaccacatggtccttcatgagtctgtaaatgctgctgggattacaggtggaggaggttctggaggcggtggaagtggtggcggaggtagcgcccagatctacagggtcacttggttcatctcctggagcccctgcttctcctggggctgtgccggggaagtgcgtgcgttccttcaggagaacacacacgtgagactgcgtatcttcgctgcccgcatctatgattacgaccccctatataaggaggcactgcaaatgctgcgggatgctggggcccaagtctccatcatgacctacgatgaatttaagcactgctgggacacctttgtggaccaccagggatgtcccttccagccctgggatggactagatgagcacagccaagccctgagtgggaggctgcgggccattctccagaatcagggaaacggcaccgaaaacctgtattttcagggctcgacaatggatcatcaccatcaccatcaccatcactaacctaggctgctaaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaa

SEQ ID NO: 46 - DNA, 5874 bp, Artificial Sequence
  A3A-INS2 pLEXm
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattgcggccgtctcaggccgaattcgccaccatggaagccagcccagcatccgggcccagacacttgatggatccacacatattcacttccaactttaacaatggcattggaaggcataagacctacctgtgctacgaagtggagcgcctggacaatggcacctcggtcaagatggaccagcacaggggctttctacacaaccaggctaagaatcttctctgtggcttttacggccgccatgcggagctgcgcttcttggacctggttccttctttgcagttggacccgggatccgctggctccgctgctggttctggcgaattcatgagcaaaggagaagaacttttcactggagttgtcccaattcttgttgaattagatggtgatgttaatgggcacaaattttctgtcagaggagagggtgaaggtgatgctacaatcggaaaactcacccttaaatttatttgcactactggaaaactacctgttccatggccaacacttgtcactactctgacctatggtgttcaatgcttttcccgttatccggatcacatgaaaaggcatgactttttcaagagtgccatgcccgaaggttatgtacaggaacgcactatatctttcaaagatgacgggaaatacaagacgcgtgctgtagtcaagtttgaaggtgatacccttgttaatcgtatcgagttaaagggtactgattttaaagaagatggaaacattctcggacacaaactcgagtacaactttaactcacacaatgtatacatcacggcagacaaacaaaagaatggaatcaaagctaacttcacagttcgccacaacgttgaagatggttccgttcaactagcagaccattatcaacaaaatactccaattggcgatggccctgtccttttaccagacaaccattacctgtcaacacaaactgtcctttcgaaagatcccaacgaaaagggtacccgtgaccacatggtccttcatgagtctgtaaatgctgctgggattacaggtggaggaggttctggaggcggtggaagtggtggcggaggtagcgcccagatctacagggtcacttggttcatctcctggagcccctgcttctcctggggctgtgccggggaagtgcgtgcgttccttcaggagaacacacacgtgagactgcgtatcttcgctgcccgcatctatgattacgaccccctatataaggaggcactgcaaatgctgcgggatgctggggcccaagtctccatcatgacctacgatgaatttaagcactgctgggacacctttgtggaccaccagggatgtcccttccagccctgggatggactagatgagcacagccaagccctgagtgggaggctgcgggccattctccagaatcagggaaactagctcgagactagtatcgcgataattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatcgatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggtc

SEQ ID NO: 47 - DNA, 5874 bp, Artificial Sequence
  A3A(E72A)-INS2 pLEXm
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattgcggccgtctcaggccgaattcgccaccatggaagccagcccagcatccgggcccagacacttgatggatccacacatattcacttccaactttaacaatggcattggaaggcataagacctacctgtgctacgaagtggagcgcctggacaatggcacctcggtcaagatggaccagcacaggggctttctacacaaccaggctaagaatcttctctgtggcttttacggccgccatgcggcgctgcgcttcttggacctggttccttctttgcagttggacccgggatccgctggctccgctgctggttctggcgaattcatgagcaaaggagaagaacttttcactggagttgtcccaattcttgttgaattagatggtgatgttaatgggcacaaattttctgtcagaggagagggtgaaggtgatgctacaatcggaaaactcacccttaaatttatttgcactactggaaaactacctgttccatggccaacacttgtcactactctgacctatggtgttcaatgcttttcccgttatccggatcacatgaaaaggcatgactttttcaagagtgccatgcccgaaggttatgtacaggaacgcactatatctttcaaagatgacgggaaatacaagacgcgtgctgtagtcaagtttgaaggtgatacccttgttaatcgtatcgagttaaagggtactgattttaaagaagatggaaacattctcggacacaaactcgagtacaactttaactcacacaatgtatacatcacggcagacaaacaaaagaatggaatcaaagctaacttcacagttcgccacaacgttgaagatggttccgttcaactagcagaccattatcaacaaaatactccaattggcgatggccctgtccttttaccagacaaccattacctgtcaacacaaactgtcctttcgaaagatcccaacgaaaagggtacccgtgaccacatggtccttcatgagtctgtaaatgctgctgggattacaggtggaggaggttctggaggcggtggaagtggtggcggaggtagcgcccagatctacagggtcacttggttcatctcctggagcccctgcttctcctggggctgtgccggggaagtgcgtgcgttccttcaggagaacacacacgtgagactgcgtatcttcgctgcccgcatctatgattacgaccccctatataaggaggcactgcaaatgctgcgggatgctggggcccaagtctccatcatgacctacgatgaatttaagcactgctgggacacctttgtggaccaccagggatgtcccttccagccctgggatggactagatgagcacagccaagccctgagtgggaggctgcgggccattctccagaatcagggaaactagctcgagactagtatcgcgataattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatcgatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggtc

SEQ ID NO: 48 - DNA, 5442 bp, Artificial Sequence
  A3A-SPL2N pLEXm
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattgcggccgtctcaggccgaattcgccaccatggaagccagcccagcatccgggcccagacacttgatggatccacacatattcacttccaactttaacaatggcattggaaggcataagacctacctgtgctacgaagtggagcgcctggacaatggcacctcggtcaagatggaccagcacaggggctttctacacaaccaggctaagaatcttctctgtggcttttacggccgccatgcggagctgcgcttcttggacctggttccttctttgcagttggacccgggatccgctggctccgctgctggttctggcgaattcatgagcaaaggagaagaacttttcactggagttgtcccaattcttgttgaattagatggtgatgttaatgggcacaaattttctgtcagaggagagggtgaaggtgatgctacaatcggaaaactcacccttaaatttatttgcactactggaaaactacctgttccatggccaacacttgtcactactctgacctatggtgttcaatgcttttcccgttatccggatcacatgaaaaggcatgactttttcaagagtgccatgcccgaaggttatgtacaggaacgcactatatctttcaaagatgacgggaaatacaagacgcgtgctgtagtcaagtttgaaggtgatacccttgttaatcgtatcgagttaaagggtactgattttaaagaagatggaaacattctcggacacaaactcgagtacaactttaactcacacaatgtatacatcacggcagacaaacaaaagaatggaatcaaagctaacttcacagttcgccacaacgttgaagatggttccgttcaactagcagaccattatcaacaaaatactccaattggcgatggccctgtccttttaccagacaaccattacctgtcaacacaaactgtcctttcgaaagatcccaacgaaaagggtacctagctcgagactagtatcgcgataattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatcgatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggtc

SEQ ID NO: 49 - DNA, 4910 bp, Artificial Sequence
  A3A-SPL2C pLEXm
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattgcggccgtctcaggccgaattcgccaccatgggtacccgtgaccacatggtccttcatgagtctgtaaatgctgctgggattacaggtggaggaggttctggaggcggtggaagtggtggcggaggtagcgcccagatctacagggtcacttggttcatctcctggagcccctgcttctcctggggctgtgccggggaagtgcgtgcgttccttcaggagaacacacacgtgagactgcgtatcttcgctgcccgcatctatgattacgaccccctatataaggaggcactgcaaatgctgcgggatgctggggcccaagtctccatcatgacctacgatgaatttaagcactgctgggacacctttgtggaccaccagggatgtcccttccagccctgggatggactagatgagcacagccaagccctgagtgggaggtagctcgagactagtatcgcgataattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatcgatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggtc

SEQ ID NO: 50 - DNA, 8961 bp, Artificial Sequence
  evoA1-BE4max
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgaaacggacagccgacggaagcgagttcgagtcaccaaagaagaagcggaaagtcagttcaaagactgggcctgtcgccgtcgatccaaccctgcgccgccggattgaacctcacgagtttgaagtgttctttgacccccgggagctgagaaaggagacatgcctgctgtacgagatcaactggggaggcaggcactccatctggaggcacacctctcagaacacaaataagcacgtggaggtgaacttcatcgagaagtttaccacagagcggtacttctgccccaataccagatgtagcatcacatggtttctgagctggtccccttgcggagagtgtagcagggccatcaccgagttcctgtccagatatccaaatgtgacactgtttatctacatcgccaggctgtatcacctggcaaacccaaggaataggcagggcctgcgcgatctgatcagctccggcgtgaccatccagatcatgacagagcaggagtccggctactgctggcacaacttcgtgaattattctcctagcaacgagtcccactggcctaggtacccacacctgtgggtgcgcctgtacgtgctggagctgtattgcatcatcctgggcctgcccccttgtctgaatatcctgcggagaaagcagagccagctgacctcctttacaatcgccctgcagtcttgtcactatcagaggctgccaccccacatcctgtgggccacaggcctgaagtctggcggatctagcggaggatcctctggcagcgagacacccgggacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcgggaagaggcggtttgcgtattgggcgcacttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaaaatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgac

SEQ ID NO: 51 - DNA, 5424 bp, Artificial Sequence
  AID′-BE4max
gtcagatccgctagagatccgcggccgctaatacgactcactatagggagagccgccaccatggatagcctgctgatgaaccgtcgtgaatttctgtatcagtttaaaaacgtgcgttgggcgaaaggccgtcgtgaaacctatctgtgctatgtggtgaaacgtcgtgatagcgcgaccagctttagcctggattttggctatctgcgtaacaaaaacggctgccatgtggaactgctgtttctgcgttatattagcgattgggatctggatccgggccgttgctatcgtgtgacctggtttatcagctggagcccgtgctatgattgcgcgcgtcatgtggcggattttctgcgtggcaacccgaacctgagcctgcgtatttttaccgcgcgtctgtatttttgcgaagccggcaggcgtgaaccggaaggcctgcgtcgtctgcatcgtgcgggcgtgcagattgcgattatgacctttaaagattatttttattgctggaacacctttgtggaaaaccatggacgtacctttaaagcgtgggaaggcctgcatgaaaacagcgtgcgtctgagccgtcagctgcgtcgtattctgctgtctggcggatctagcggaggatcctctggcagcgagacacccgggacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaa

SEQ ID NO: 52 - DNA, 5478 bp, Artificial Sequence
  A3A-BE4max
gtcagatccgctagagatccgcggccgctaatacgactcactatagggagagccgccaccatggaagccagcccagcatccgggcccagacacttgatggatccacacatattcacttccaactttaacaatggcattggaaggcataagacctacctgtgctacgaagtggagcgcctggacaatggcacctcggtcaagatggaccagcacaggggctttctacacaaccaggctaagaatcttctctgtggcttttacggccgccatgcggagctgcgcttcttggacctggttccttctttgcagttggacccggcccagatctacagggtcacttggttcatctcctggagcccctgcttctcctggggctgtgccggggaagtgcgtgcgttccttcaggagaacacacacgtgagactgcgtatcttcgctgcccgcatctatgattacgaccccctatataaggaggcactgcaaatgctgcgggatgctggggcccaagtctccatcatgacctacgatgaatttaagcactgctgggacacctttgtggaccaccagggatgtcccttccagccctgggatggactagatgagcacagccaagccctgagtgggaggctgcgggccattctccagaatcagggaaactctggcggatctagcggaggatcctctggcagcgagacacccgggacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaa

SEQ ID NO: 53 - DNA, 6438 bp, Artificial Sequence
  evoA1-seBE4max
gtcagatccgctagagatccgcggccgctaatacgactcactatagggagagccgccaccatggaacaaaaacttatttctgaagaagatctgaaaaggccggcggccacgaaaaaggccggccaggcaaaaaagaaaaagggaggttccgctagcggaggttcgatgagttcaaagactgggcctgtcgccgtcgatccaaccctgcgccgccggattgaacctcacgagtttgaagtgttctttgacccccgggagctgagaaaggagacatgcctgctgtacgagatcaactggggaggcaggcactccatctggaggcacacctctcagaacacaaataagcacgtggaggtgaacttcatcgagaagtttaccacagagcggtacttctgccccggcgcgccgggaggtggtggcagcggtggaggaggttctgggggcggtggctcaattttatggcatgagatgtggcatgagggtttggaagaggcatctagattgtatttcggtgaaagaaatgtcaagggaatgttcgaagttttagaaccgttgcacgctatgatggagagaggtccacagactctaaaggagacttccttcaaccaagcttatggaagggacctaatggaggctcaagaatggtgtagaaaatacatgaaaagtggaaatgtaaaggaccttacacaagcttgggatctctactaccatgtttttaggagaatatctaaaggaagtggtgagggtaggggaagtttattaacctgtggggatgttgaagaaaatccaggtcctatgggcgtacaagttgaaactatcagccctggggacggcagaacctttccgaagaggggacagacatgtgttgttcactatactggaatgttggaagatggtaagaagttcgatagcagcagagataggaataaaccatttaaattcatgcttggcaagcaagaagtgattaggggttgggaagaaggtgtcgctcaaatgagtgtaggtcagagggctaagttaacaattagtcctgattatgcttatggcgctacaggtcatccaggaatcattcccccacatgctactcttgttttcgacgttgaattgcttaagcttgaaggatcaggttctggatctggttcaggatcaggctcacccgggcttaataccagatgtagcatcacatggtttctgagctggtccccttgcggagagtgtagcagggccatcaccgagttcctgtccagatatccaaatgtgacactgtttatctacatcgccaggctgtatcacctggcaaacccaaggaataggcagggcctgcgcgatctgatcagctccggcgtgaccatccagatcatgacagagcaggagtccggctactgctggcacaacttcgtgaattattctcctagcaacgagtcccactggcctaggtacccacacctgtgggtgcgcctgtacgtgctggagctgtattgcatcatcctgggcctgcccccttgtctgaatatcctgcggagaaagcagagccagctgacctcctttacaatcgccctgcagtcttgtcactatcagaggctgccaccccacatcctgtgggccacaggcctgaagtctggcggatctagcggaggatcctctggcagcgagacacccgggacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaa

SEQ ID NO: 54 - DNA, 6294 bp, Artificial Sequence
  AID′-seBE4max
gtcagatccgctagagatccgcggccgctaatacgactcactatagggagagccgccaccatggaacaaaaacttatttctgaagaagatctgaaaaggccggcggccacgaaaaaggccggccaggcaaaaaagaaaaagggaggttccgctagcggaggttcgatggatagcctgctgatgaaccgtcgtgaatttctgtatcagtttaaaaacgtgcgttgggcgaaaggccgtcgtgaaacctatctgtgctatgtggtgaaacgtcgtgatagcgcgaccagctttagcctggattttggctatctgcgtaacaaaaacggctgccatgtggaactgctgtttctgcgttatattagcgattgggatctggatccgggcgcgccgggaggtggtggcagcggtggaggaggttctgggggcggtggctcaattttatggcatgagatgtggcatgagggtttggaagaggcatctagattgtatttcggtgaaagaaatgtcaagggaatgttcgaagttttagaaccgttgcacgctatgatggagagaggtccacagactctaaaggagacttccttcaaccaagcttatggaagggacctaatggaggctcaagaatggtgtagaaaatacatgaaaagtggaaatgtaaaggaccttacacaagcttgggatctctactaccatgtttttaggagaatatctaaaggaagtggtgagggtaggggaagtttattaacctgtggggatgttgaagaaaatccaggtcctatgggcgtacaagttgaaactatcagccctggggacggcagaacctttccgaagaggggacagacatgtgttgttcactatactggaatgttggaagatggtaagaagttcgatagcagcagagataggaataaaccatttaaattcatgcttggcaagcaagaagtgattaggggttgggaagaaggtgtcgctcaaatgagtgtaggtcagagggctaagttaacaattagtcctgattatgcttatggcgctacaggtcatccaggaatcattcccccacatgctactcttgttttcgacgttgaattgcttaagcttgaaggatcaggttctggatctggttcaggatcaggctcacccgggcttggccgttgctatcgtgtgacctggtttatcagctggagcccgtgctatgattgcgcgcgtcatgtggcggattttctgcgtggcaacccgaacctgagcctgcgtatttttaccgcgcgtctgtatttttgcgaagccggcaggcgtgaaccggaaggcctgcgtcgtctgcatcgtgcgggcgtgcagattgcgattatgacctttaaagattatttttattgctggaacacctttgtggaaaaccatggacgtacctttaaagcgtgggaaggcctgcatgaaaacagcgtgcgtctgagccgtcagctgcgtcgtattctgctgtctggcggatctagcggaggatcctctggcagcgagacacccgggacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaa

SEQ ID NO: 55 - DNA, 6348 bp, Artificial Sequence
  A3A-seBE4max
gtcagatccgctagagatccgcggccgctaatacgactcactatagggagagccgccaccatggaacaaaaacttatttctgaagaagatctgaaaaggccggcggccacgaaaaaggccggccaggcaaaaaagaaaaagggaggttccgctagcggaggttcgatggaagccagcccagcatccgggcccagacacttgatagatccacacatattcacttccaactttaacaatggcattggaaggcataagacctacctgtgctacgaagtggagcgcctggacaatggcacctcggtcaagatggaccagcacaggggctttctacacaaccaggctaagaatcttctctgtggcttttacggccgccatgcggagctgcgcttcttggacctggttccttctttgcagttggacccgggcgcgccgggaggtggtggcagcggtggaggaggttctgggggcggtggctcaattttatggcatgagatgtggcatgagggtttggaagaggcatctagattgtatttcggtgaaagaaatgtcaagggaatgttcgaagttttagaaccgttgcacgctatgatggagagaggtccacagactctaaaggagacttccttcaaccaagcttatggaagggacctaatggaggctcaagaatggtgtagaaaatacatgaaaagtggaaatgtaaaggaccttacacaagcttgggatctctactaccatgtttttaggagaatatctaaaggaagtggtgagggtaggggaagtttattaacctgtggggatgttgaagaaaatccaggtcctatgggcgtacaagttgaaactatcagccctggggacggcagaacctttccgaagaggggacagacatgtgttgttcactatactggaatgttggaagatggtaagaagttcgatagcagcagagataggaataaaccatttaaattcatgcttggcaagcaagaagtgattaggggttgggaagaaggtgtcgctcaaatgagtgtaggtcagagggctaagttaacaattagtcctgattatgcttatggcgctacaggtcatccaggaatcattcccccacatgctactcttgttttcgacgttgaattgcttaagcttgaaggatcaggttctggatctggttcaggatcaggctcacccgggcttgcccagatctacagggtcacctggttcatctcctggagcccctgcttctcctggggctgtgccggggaagtgcgtgcgttcctgcaggagaacacacacgtgagactgcgtatcttcgctgcccgcatctatgattacgaccccctatataaggaggcactgcaaatgctgcgggatgctggggcccaagtctccatcatgacctacgatgaatttaagcactgctgggacacctttgtggaccaccagggatgtcccttccagccctgggatggactagatgagcacagccaagccctgagtgggaggctgcgggccattctccagaatcagggaaactctggcggatctagcggaggatcctctggcagcgagacacccgggacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaa

SEQ ID NO: 56 - DNA, 6966 bp, Artificial Sequence
  evoA1-seBE4max-IRES
gtcagatccgctagagatccgcggccgctaatacgactcactatagggagagccgccaccatggaacaaaaacttatttctgaagaagatctgaaaaggccggcggccacgaaaaaggccggccaggcaaaaaagaaaaagggaggttccgctagcggaggttcgatgagttcaaagactgggcctgtcgccgtcgatccaaccctgcgccgccggattgaacctcacgagtttgaagtgttctttgacccccgggagctgagaaaggagacatgcctgctgtacgagatcaactggggaggcaggcactccatctggaggcacacctctcagaacacaaataagcacgtggaggtgaacttcatcgagaagtttaccacagagcggtacttctgccccggcgcgccgggaggtggtggcagcggtggaggaggttctgggggcggtggctcaattttatggcatgagatgtggcatgagggtttggaagaggcatctagattgtatttcggtgaaagaaatgtcaagggaatgttcgaagttttagaaccgttgcacgctatgatggagagaggtccacagactctaaaggagacttccttcaaccaagcttatggaagggacctaatggaggctcaagaatggtgtagaaaatacatgaaaagtggaaatgtaaaggaccttacacaagcttgggatctctactaccatgtttttaggagaatatctaaataatgaccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggcccccaagcttgccaccatgggcgtacaagttgaaactatcagccctggggacggcagaacctttccgaagaggggacagacatgtgttgttcactatactggaatgttggaagatggtaagaagttcgatagcagcagagataggaataaaccatttaaattcatgcttggcaagcaagaagtgattaggggttgggaagaaggtgtcgctcaaatgagtgtaggtcagagggctaagttaacaattagtcctgattatgcttatggcgctacaggtcatccaggaatcattcccccacatgctactcttgttttcgacgttgaattgcttaagcttgaaggatcaggttctggatctggttcaggatcaggctcacccgggcttaataccagatgtagcatcacatggtttctgagctggtccccttgcggagagtgtagcagggccatcaccgagttcctgtccagatatccaaatgtgacactgtttatctacatcgccaggctgtatcacctggcaaacccaaggaataggcagggcctgcgcgatctgatcagctccggcgtgaccatccagatcatgacagagcaggagtccggctactgctggcacaacttcgtgaattattctcctagcaacgagtcccactggcctaggtacccacacctgtgggtgcgcctgtacgtgctggagctgtattgcatcatcctgggcctgcccccttgtctgaatatcctgcggagaaagcagagccagctgacctcctttacaatcgccctgcagtcttgtcactatcagaggctgccaccccacatcctgtgggccacaggcctgaagtctggcggatctagcggaggatcctctggcagcgagacacccgggacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaa

SEQ ID NO: 57 - DNA, 9310 bp, Artificial Sequence
  LRcherry 2.1 empty
ttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccagagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacggttgtaaatgagcacacaaaatacacatgctaaaatattatattctatgacctttataaaatcaaccaaaatcttctttttaataactttagtatcaataattagaatttttatgttcctttttgcaaacttttaataaaaatgagcaaaataaaaaaacgctagttttagtaactcgcgttgttttcttcacctttaataatagctactccaccacttgttcctaagcggtcagctcctgcttcaatcattttttgagcatcttcaaatgttctaactccaccagctgctttaactaaagcattgtctttaacaactgacttcattagtttaacatcttcaaatgttgcacctgattttgaaaatcctgttgatgttttaacaaattctaatccagcttcaacagctatttcacaagctttcatgatttcttcttttgttaataaacaattttccataatacatttaacaacatgtgatccagctgctttttttacagctttcatgtcttctaaaactaattcataatttttgtcttttaatgcaccaatatttaataccatatcaatttctgttgcaccatctttaattgcttcagaaacttcgaatgcttttgtagctgttgtgcatgcacctagaggaaaacctacaacatttgttattcctacatttgtgccttttaataattctttacaatagcttgttcaatatgaattaacacaaactgttgcaaaatcaaattcaattgcttcatcacataattgtttaatttcagctttcgtagcatcttgttttaataatgtgtgatctatatatttgtttagtttcattttttctcctatatattcatttttaattttaattctttaataatttcgtctactttaactttagcgttttgaacagattcaccaacacctataaaataaatttttagtttaggttcagttccacttgggcgaacagcaaatcatgacttatcttctaaataaaattttagtaagtcttgtcctggcatattatacattccatcgatgtagtcttcaacattaacaactttaagtccagcaatttgagttaagggtgttgctctcaatgatttcattaatggttcaatttttaatttcttttcttctggtttaaaattcaagtttaaagtgaaagtgtaatatgcacccatttctttaaataaatcttctaaatagtctactaatgttttattttgttttttataaaatcaagcagcctctgctattaatatagaagcttgtattccatctttatctctagctgagtcatcaattacatatccataactttcttcataagcaaaaacaaaatttaatccgttatcttcttctttagcaatttctctacccattcatttaaatccagttaaagtttttacaatattaactccatatttttcatgagcgattctatcacccaaatcacttgttacaaaacttgaatatagagccggattttttggaatgctatttaagcgttttagatttgataattttcaatcaattaaaattggtcctgtttgatttccatctaatcttacaaaatgaccatcatgttttattgccattccaaatctgtcagcatctgggtcattcataataataatatctgcatcatgtttaataccatattcaagcggtatttttcatgcaggatcaaattctggatttggatttacaacatttttaaatgtttcatcttcaaatgcatgctcttcaacctcaataacgttatatcctgattcacgtaatatttttggggtaaatttagttcctgttccattaactgcgctaaaaataatttttaaatcttttttagcttcttgctcttttttgtacgtctctgtttaagagctatgctggaaacagcatagcaagtttaaataaggctagtccgttatcaactttgctggaaacagcaaagtggcaccgagtcggtgctttttttgaattctagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcgggatccgccaccatggattacaaagacgatgacgataagatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagcgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagc

SEQ ID NO: 58 - DNA, 9318 bp, Artificial Sequence
  LRG empty
ttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccagagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacggttgtaaatgagcacacaaaatacacatgctaaaatattatattctatgacctttataaaatcaaccaaaatcttctttttaataactttagtatcaataattagaatttttatgttcctttttgcaaacttttaataaaaatgagcaaaataaaaaaacgctagttttagtaactcgcgttgttttcttcacctttaataatagctactccaccacttgttcctaagcggtcagctcctgcttcaatcattttttgagcatcttcaaatgttctaactccaccagctgctttaactaaagcattgtctttaacaactgacttcattagtttaacatcttcaaatgttgcacctgattttgaaaatcctgttgatgttttaacaaattctaatccagcttcaacagctatttcacaagctttcatgatttcttcttttgttaataaacaattttccataatacatttaacaacatgtgatccagctgctttttttacagctttcatgtcttctaaaactaattcataatttttgtcttttaatgcaccaatatttaataccatatcaatttctgttgcaccatctttaattgcttcagaaacttcgaatgcttttgtagctgttgtgcatgcacctagaggaaaacctacaacatttgttattcctacatttgtgccttttaataattctttacaatagcttgttcaatatgaattaacacaaactgttgcaaaatcaaattcaattgcttcatcacataattgtttaatttcagctttcgtagcatcttgttttaataatgtgtgatctatatatttgtttagtttcattttttctcctatatattcatttttaattttaattctttaataatttcgtctactttaactttagcgttttgaacagattcaccaacacctataaaataaatttttagtttaggttcagttccacttgggcgaacagcaaatcatgacttatcttctaaataaaattttagtaagtcttgtcctggcatattatacattccatcgatgtagtcttcaacattaacaactttaagtccagcaatttgagttaagggtgttgctctcaatgatttcattaatggttcaatttttaatttcttttcttctggtttaaaattcaagtttaaagtgaaagtgtaatatgcacccatttctttaaataaatcttctaaatagtctactaatgttttattttgttttttataaaatcaagcagcctctgctattaatatagaagcttgtattccatctttatctctagctgagtcatcaattacatatccataactttcttcataagcaaaaacaaaatttaatccgttatcttcttctttagcaatttctctacccattcatttaaatccagttaaagtttttacaatattaactccatatttttcatgagcgattctatcacccaaatcacttgttacaaaacttgaatatagagccggattttttggaatgctatttaagcgttttagatttgataattttcaatcaattaaaattggtcctgtttgatttccatctaatcttacaaaatgaccatcatgttttattgccattccaaatctgtcagcatctgggtcattcataataataatatctgcatcatgtttaataccatattcaagcggtatttttcatgcaggatcaaattctggatttggatttacaacatttttaaatgtttcatcttcaaatgcatgctcttcaacctcaataacgttatatcctgattcacgtaatatttttggggtaaatttagttcctgttccattaactgcgctaaaaataatttttaaatcttttttagcttcttgctcttttttgtacgtctctgtttaagagctatgctggaaacagcatagcaagtttaaataaggctagtccgttatcaactttgctggaaacagcaaagtggcaccgagtcggtgctttttttgaattctagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcgggatccgccaccatggattacaaagacgatgacgataagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagcgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagc